SEQUENCE LISTING (126 sequences)

SEQ ID NO: 1 - RNA, 61 nt, synthetic construct
  source 1..61: mol_type = other RNA
agggaaataagagagaaaagaagagtaagaagaaatataagaccccggcgccgccaccatg

SEQ ID NO: 2 - RNA, 60 nt, synthetic construct
  source 1..60: mol_type = other RNA
gggaaataagagagaaaagaagagtaagaagaaatataagaccccggcgccgccaccatg

SEQ ID NO: 3 - RNA, 60 nt, synthetic construct; identical to SEQ ID NO: 2
  source 1..60: mol_type = other RNA

SEQ ID NO: 4 - RNA, 60 nt, synthetic construct; identical to SEQ ID NO: 2
  source 1..60: mol_type = other RNA

SEQ ID NO: 5 - RNA, 105 nt, synthetic construct
  source 1..105: mol_type = other RNA
gggaacaaatgcactgaaacaaatgcactgaaacaaatgcactgagggaaataagagagaaaagaagagtaagaagaaatataagaccccggcgccgccaccatg

SEQ ID NO: 6 - RNA, 126 nt, synthetic construct
  source 1..126: mol_type = other RNA
gggaacaattgcactgaaacaaatgcactgaaacaaatgcactgaaacaaatgcactgagggaaataagtgcactgaaaataagtgcactgaaaataagtgcactgaccccggcgccgccaccatg

SEQ ID NO: 7 - RNA, 130 nt, synthetic construct
  source 1..130: mol_type = other RNA
aggaacaatctgcactgaaacaaactgcactgaaacaaactgcactgaaacaaactgcactgagggaaataagtgcactgaaaataagtgcactgaaaataagtgcactgaccccggcgccgccaccatg

SEQ ID NO: 8 - RNA, 81 nt, synthetic construct
  source 1..81: mol_type = other RNA
aggaaaagtgcactgaatttagggaaataagagagaaaagaagagtaagaagaaatataagaccccggcgccgccaccatg

SEQ ID NO: 9 - RNA, 83 nt, synthetic construct
  source 1..83: mol_type = other RNA
aggaaaagagtgcactgaatttagggaaataagagagaaaagaagagtaagaagaaatataagaccccggcgccgccaccatg

SEQ ID NO: 10 - RNA, 98 nt, synthetic construct
  source 1..98: mol_type = other RNA
aggaaaagagtgcactgaatttaaaaagagtgcactgagggaaataagagagaaaagaagagtaagaagaaatataagaccccggcgccgccaccatg

SEQ ID NO: 11 - RNA, 103 nt, synthetic construct
  source 1..103: mol_type = other RNA
aggaaaagagtgcactgaatttaaaaagagtgcactgaatttagggaaataagagagaaaagaagagtaagaagaaatataagaccccggcgccgccaccatg

SEQ ID NO: 12 - RNA, 118 nt, synthetic construct
  source 1..118: mol_type = other RNA
aggaaaagagtgcactgaatttaaaaagagtgcactgaatttaaaaagagtgcactgagggaaataagagagaaaagaagagtaagaagaaatataagaccccggcgccgccaccatg

SEQ ID NO: 13 - RNA, 114 nt, synthetic construct
  source 1..114: mol_type = other RNA
aggaataaactgcactgaaataaactgcactgaaataaactgcactgaaataaagggaaataagagagaaaagaagagtaagaagaaatataagaccccggcgccgccaccatg

SEQ ID NO: 14 - RNA, 84 nt, synthetic construct
  source 1..84: mol_type = other RNA
aggaataaactgcactgaaataaagggaaataagagagaaaagaagagtaagaagaaatataagaccccggcgccgccaccatg

SEQ ID NO: 15 - RNA, 60 nt, synthetic construct; identical to SEQ ID NO: 2
  source 1..60: mol_type = other RNA

SEQ ID NO: 16-31 - skipped (16 entries)

SEQ ID NO: 32 - DNA, 68 bp, Homo sapiens
  source 1..68: mol_type = other DNA
gaggcaaagttctgagacactccgactctgagtatgatagaagtcagtgcactacagaactttgtctc

SEQ ID NO: 33 - RNA, 68 nt, Homo sapiens
  source 1..68: mol_type = mRNA
gaggcaaagttctgagacactccgactctgagtatgatagaagtcagtgcactacagaactttgtctc

SEQ ID NO: 34-39 - skipped (6 entries)

SEQ ID NO: 40 - RNA, 110 nt, synthetic construct
  source 1..110: mol_type = other RNA
aggaacaaagtataagctaaacaaagtataagctaaacagagtataagctagggaaataagagagaaaagaagagtaagaagaaatataagaccccggcttcgccaccat

SEQ ID NO: 41 - RNA, 80 nt, synthetic construct
  source 1..80: mol_type = other RNA
aggaacaaagtataagctagggaaataagagagaaaagaagagtaagaagaaatataagaccccggcttcagccaccatg

SEQ ID NO: 42 - RNA, 113 nt, synthetic construct
  source 1..113: mol_type = other RNA
aggaacaaacataggtcagaacaaacataggtcagaacaaacataggtcaggggaaataagagagaaaagaagagtaagaagaaatataagaccccggcgccttgccaccatg

SEQ ID NO: 43 - RNA, 78 nt, synthetic construct
  source 1..78: mol_type = other RNA
aggaacaaacataggtcaggggaaataagagagaaaagaagagtaagaagaaatataagacccgcgcctgccaccatg

SEQ ID NO: 44 - RNA, 113 nt, synthetic construct
  source 1..113: mol_type = other RNA
aggaacaaacataggtcagaacaaacataggtcagaacagatacaatgcacgggaaataagagagaaaagaagagtaagaagaaatataagaccccgtcgccttgccaccatg

SEQ ID NO: 45 - RNA, 113 nt, synthetic construct; identical to SEQ ID NO: 44
  source 1..113: mol_type = other RNA

SEQ ID NO: 46-51 - skipped (6 entries)

SEQ ID NO: 52 - protein, 2261 aa, Homo sapiens
MACWPQLRLLLWKNLTFRRRQTCQLLLEVAWPLFIFLILISVRLSYPPYEQHECHFPNKAMPSAGTLPWVQGIICNANNPCFRYPTPGEAPGVVGNFNKSIVARLFSDARRLLLYSQKDTSMKDMRKVLRTLQQIKKSSSNLKLQDFLVDNETFSGFLYHNLSLPKSTVDKMLRADVILHKVFLQGYQLHLTSLCNGSKSEEMIQLGDQEVSELCGLPREKLAAAERVLRSNMDILKPILRTLNSTSPFPSKELAEATKTLLHSLGTLAQELFSMRSWSDMRQEVMFLTNVNSSSSSTQIYQAVSRIVCGHPEGGGLKIKSLNWYEDNNYKALFGGNGTEEDAETFYDNSTTPYCNDLMKNLESSPLSRIIWKALKPLLVGKILYTPDTPATRQVMAEVNKTFQELAVFHDLEGMWEELSPKIWTFMENSQEMDLVRMLLDSRDNDHFWEQQLDGLDWTAQDIVAFLAKHPEDVQSSNGSVYTWREAFNETNQAIRTISRFMECVNLNKLEPIATEVWLINKSMELLDERKFWAGIVFTGITPGSIELPHHVKYKIRMDIDNVERTNKIKDGYWDPGPRADPFEDMRYVWGGFAYLQDVVEQAIIRVLTGTEKKTGVYMQQMPYPCYVDDIFLRVMSRSMPLFMTLAWIYSVAVIIKGIVYEKEARLKETMRIMGLDNSILWFSWFISSLIPLLVSAGLLVVILKLGNLLPYSDPSVVFVFLSVFAVVTILQCFLISTLFSRANLAAACGGIIYFTLYLPYVLCVAWQDYVGFTLKIFASLLSPVAFGFGCEYFALFEEQGIGVQWDNLFESPVEEDGFNLTTSVSMMLFDTFLYGVMTWYIEAVFPGQYGIPRPWYFPCTKSYWFGEESDEKSHPGSNQKRISEICMEEEPTHLKLGVSIQNLVKVYRDGMKVAVDGLALNFYEGQITSFLGHNGAGKTTTMSILTGLFPPTSGTAYILGKDIRSEMSTIRQNLGVCPQHNVLFDMLTVEEHIWFYARLKGLSEKHVKAEMEQMALDVGLPSSKLKSKTSQLSGGMQRKLSVALAFVGGSKVVILDEPTAGVDPYSRRGIWELLLKYRQGRTIILSTHHMDEADVLGDRIAIISHGKLCCVGSSLFLKNQLGTGYYLTLVKKDVESSLSSCRNSSSTVSYLKKEDSVSQSSSDAGLGSDHESDTLTIDVSAISNLIRKHVSEARLVEDIGHELTYVLPYEAAKEGAFVELFHEIDDRLSDLGISSYGISETTLEEIFLKVAEESGVDAETSDGTLPARRNRRAFGDKQSCLRPFTEDDAADPNDSDIDPESRETDLLSGMDGKGSYQVKGWKLTQQQFVALLWKRLLIARRSRKGFFAQIVLPAVFVCIALVFSLIVPPFGKYPSLELQPWMYNEQYTFVSNDAPEDTGTLELLNALTKDPGFGTRCMEGNPIPDTPCQAGEEEWTTAPVPQTIMDLFQNGNWTMQNPSPACQCSSDKIKKMLPVCPPGAGGLPPPQRKQNTADILQDLTGRNISDYLVKTYVQIIAKSLKNKIWVNEFRYGGFSLGVSNTQALPPSQEVNDAIKQMKKHLKLAKDSSADRFLNSLGRFMTGLDTKNNVKVWFNNKGWHAISSFLNVINNAILRANLQKGENPSHYGITAFNHPLNLTKQQLSEVALMTTSVDVLVSICVIFAMSFVPASFVVFLIQERVSKAKHLQFISGVKPVIYWLSNFVWDMCNYVVPATLVIIIFICFQQKSYVSSTNLPVLALLLLLYGWSITPLMYPASFVFKIPSTAYVVLTSVNLFIGINGSVATFVLELFTDNKLNNINDILKSVFLIFPHFCLGRGLIDMVKNQAMADALERFGENRFVSPLSWDLVGRNLFAMAVEGVVFFLITVLIQYRFFIRPRPVNAKLSPLNDEDEDVRRERQRILDGGGQNDILEIKELTKIYRRKRKPAVDRICVGIPPGECFGLLGVNGAGKSSTFKMLTGDTTVTRGDAFLNKNSILSNIHEVHQNMGYCPQFDAITELLTGREHVEFFALLRGVPEKEVGKVGEWAIRKLGLVKYGEKYAGNYSGGNKRKLSTAMALIGGPPVVFLDEPTTGMDPKARRFLWNCALSVVKEGRSVVLTSHSMEECEALCTRMAIMVNGRFRCLGSVQHLKNRFGDGYTIVVRIAGSNPDLKPVQDFFGLAFPGSVLKEKHRNMLQYQLPSSLSSLARIFSILSQSKKRLHIEDYSVSQTTLDQVFVNFAKDQSDDDHLKDLSLHKNQTVVDVAVLTSFLQDEKVKESYV

SEQ ID NO: 53 - protein, 860 aa, Homo sapiens [sequence omitted]

SEQ ID NO: 54-59 - skipped (6 entries)

SEQ ID NO: 60 - RNA, 10 nt, Homo sapiens
  source 1..10: mol_type = mRNA
cataggtcag

SEQ ID NO: 61-69 - skipped (9 entries)

SEQ ID NO: 70 - RNA, 10 nt, Homo sapiens
  source 1..10: mol_type = mRNA
tacaatgcac

SEQ ID NO: 71-79 - skipped (9 entries)

SEQ ID NO: 80 - RNA, 10 nt, Homo sapiens
  source 1..10: mol_type = mRNA
gtataagcta

SEQ ID NO: 81-86 - skipped (6 entries)

SEQ ID NO: 87 - RNA, 105 nt, synthetic construct
  source 1..105: mol_type = other RNA
gggaacaaatgcactgaaacaaatgcactgaaacaaatgcactgagggnnnnnnnnnnnnnnnnnnnnnnnnnnnnnnnnnnnnnnccccggcgccgccaccatg

SEQ ID NO: 88 - RNA, 108 nt, synthetic construct
  source 1..108: mol_type = other RNA
gggaacaaagtgcactgaaacaaagtgcactgaaacaaagtgcactgagggnnnnnnnnnnnnnnnnnnnnnnnnnnnnnnnnnnnnnnccccggcgccgccaccatg

SEQ ID NO: 89 - RNA, 111 nt, synthetic construct
  source 1..111: mol_type = other RNA
gggaacaaaagtgcactgaaacaaaagtgcactgaaacaaaggtgcactgagggnnnnnnnnnnnnnnnnnnnnnnnnnnnnnnnnnnnnnnccccggcgccgccaccatg

SEQ ID NO: 90 - RNA, 141 nt, synthetic construct
  source 1..141: mol_type = other RNA
gggaacaaagttctgtagtgcactgaaacaaaacaaagttctgtagtggactgaaacaaaacaaagttcgtggtgcactgagggnnnnnnnnnnnnnnnnnnnnnnnnnnnnnnnnnnnnnnccccggcgccgccaccatg

SEQ ID NO: 91 - RNA, 123 nt, synthetic construct
  source 1..123: mol_type = other RNA
gggaacaatgcactgaaaaaatgcactgaaacaaatgcactgaaacaaatgcactgagggaaataagtgcactgaaaataagtgcactgaaataagtgcactgagggcggcgcccgcaccatg

SEQ ID NO: 92 - RNA, 111 nt, synthetic construct
  source 1..111: mol_type = other RNA
aggaacaaagtataagctaaacaaagtataagctaaacagagtataagctagggaaataagagagaaaagaagagtaagaagaaatataagaccccggcttcgccaccatg

SEQ ID NO: 93 - RNA, 113 nt, synthetic construct
  source 1..113: mol_type = other RNA
ggaggaacaaagtataagctaaacaaagtataagctaaacagagtataagctagggnnnnnnnnnnnnnnnnnnnnnnnnnnnnnnnnnnnnnnccccggcttcgccaccatg

SEQ ID NO: 94 - RNA, 80 nt, synthetic construct; identical to SEQ ID NO: 41
  source 1..80: mol_type = other RNA

SEQ ID NO: 95 - RNA, 81 nt, synthetic construct
  source 1..81: mol_type = other RNA
ggaggaacaagtataagctagggnnnnnnnnnnnnnnnnnnnnnnnnnnnnnnnnnnnnnnccccggcttcagccaccatg

SEQ ID NO: 96 - RNA, 112 nt, synthetic construct
  source 1..112: mol_type = other RNA
aggaacaaacataggtcagaacaaacataggtcagaacaacataggtcaggggaaataagagagaaaagaagagtaagaagaaatataagaccccggcgccttgccaccatg

SEQ ID NO: 97 - RNA, 115 nt, synthetic construct
  source 1..115: mol_type = other RNA
ggaggaacaaacataggtcagaacaaacataggtcagaacaaacataggtcaggggnnnnnnnnnnnnnnnnnnnnnnnnnnnnnnnnnnnnnnccccggcgccttgccaccatg

SEQ ID NO: 98 - RNA, 78 nt, synthetic construct
  source 1..78: mol_type = other RNA
aggaacaaacataggacaggggaaataagagagaaaagaagagtaagaagaaatataagacccgcgcctgccaccatg

SEQ ID NO: 99 - RNA, 79 nt, synthetic construct
  source 1..79: mol_type = other RNA
ggaggaacaacataggtcagggnnnnnnnnnnnnnnnnnnnnnnnnnnnnnnnnnnnnnnncccgcgcctgccaccatg

SEQ ID NO: 100 - RNA, 113 nt, synthetic construct; identical to SEQ ID NO: 44
  source 1..113: mol_type = other RNA

SEQ ID NO: 101 - RNA, 116 nt, synthetic construct
  source 1..116: mol_type = other RNA
ggaggaacaaacataggtcagaacaaacataggtcagaacagatacaatgcacggggnnnnnnnnnnnnnnnnnnnnnnnnnnnnnnnnnnnnnnccccgtcgccttgccaccatg

SEQ ID NO: 102 - RNA, 114 nt, synthetic construct
  source 1..114: mol_type = other RNA
aggaacaaagtataagctaaacaaagtataagctaaacagatacaatgcacgggaaataagagagaaaagaagaggtaagaagaaatataagaccccgtcgttgcgccaccatg

SEQ ID NO: 103 - RNA, 116 nt, synthetic construct
  source 1..116: mol_type = other RNA
ggaggaacaaagtataagctaaacaaagtataagctaaacagatacaatgcacggggnnnnnnnnnnnnnnnnnnnnnnnnnnnnnnnnnnnnnnccccgtcgccttgccaccatg

SEQ ID NO: 104 - RNA, 113 nt, synthetic construct
  source 1..113: mol_type = other RNA
aggaacaaagtataagctaaacaaagtataagctaaacagatacaatgcacgggaaataagagagaaaagaagagtaagaagaaatataagaccccgtcgttgcgccaccatg

SEQ ID NO: 105 - RNA, 113 nt, synthetic construct; identical to SEQ ID NO: 104
  source 1..113: mol_type = other RNA

SEQ ID NO: 106 - DNA, 2580 bp, Homo sapiens
  source 1..2580: mol_type = other DNA
atggggccctggggctggaaattgcgctggaccgtcgccttgctcctcgccgcggcggggactgcagtgggcgacagatgcgaaagaaacgagttccagtgccaagacgggaaatgcatctcctacaagtgggtctgcgatggcagcgctgagtgccaggatggctctgatgagtcccaggagacgtgcttgtctgtcacctgcaaatccggggacttcagctgtgggggccgtgtcaaccgctgcattcctcagttctggaggtgcgatggccaagtggactgcgacaacggctcagacgagcaaggctgtccccccaagacgtgctcccaggacgagtttcgctgccacgatgggaagtgcatctctcggcagttcgtctgtgactcagaccgggactgcttggacggctcagacgaggcctcctgcccggtgctcacctgtggtcccgccagcttccagtgcaacagctccacctgcatcccccagctgtgggcctgcgacaacgaccccgactgcgaagatggctcggatgagtggccgcagcgctgtaggggtctttacgtgttccaaggggacagtagcccctgctcggccttcgagttccactgcctaagtggcgagtgcatccactccagctggcgctgtgatggtggccccgactgcaaggacaaatctgacgaggaaaactgcgctgtggccacctgtcgccctgacgaattccagtgctctgatggaaactgcatccatggcagccggcagtgtgaccgggaatatgactgcaaggacatgagcgatgaagttggctgcgttaatgtgacactctgcgagggacccaacaagttcaagtgtcacagcggcgaatgcatcaccctggacaaagtctgcaacatggctagagactgccgggactggtcagatgaacccatcaaagagtgcgggaccaacgaatgcttggacaacaacggcggctgttcccacgtctgcaatgaccttaagatcggctacgagtgcctgtgccccgacggcttccagctggtggcccagcgaagatgcgaagatatcgatgagtgtcaggatcccgacacctgcagccagctctgcgtgaacctggagggtggctacaagtgccagtgtgaggaaggcttccagctggacccccacacgaaggcctgcaaggctgtgggctccatcgcctacctcttcttcaccaaccggcacgaggtcaggaagatgacgctggaccggagcgagtacaccagcctcatccccaacctgaggaacgtggtcgctctggacacggaggtggccagcaatagaatctactggtctgacctgtcccagagaatgatctgcagcacccagcttgacagagcccacggcgtctcttcctatgacaccgtcatcagcagagacatccaggcccccgacgggctggctgtggactggatccacagcaacatctactggaccgactctgtcctgggcactgtctctgttgcggataccaagggcgtgaagaggaaaacgttattcagggagaacggctccaagccaagggccatcgtggtggatcctgttcatggcttcatgtactggactgactggggaactcccgccaagatcaagaaagggggcctgaatggtgtggacatctactcgctggtgactgaaaacattcagtggcccaatggcatcaccctagatctcctcagtggccgcctctactgggttgactccaaacttcactccatctcaagcatcgatgtcaacgggggcaaccggaagaccatcttggaggatgaaaagaggctggcccaccccttctccttggccgtctttgaggacaaagtattttggacagatatcatcaacgaagccattttcagtgccaaccgcctcacaggttccgatgtcaacttgttggctgaaaacctactgtccccagaggatatggttctcttccacaacctcacccagccaagaggagtgaactggtgtgagaggaccaccctgagcaatggcggctgccagtatctgtgcctccctgccccgcagatcaacccccactcgcccaagtttacctgcgcctgcccggacggcatgctgctggccagggacatgaggagctgcctcacagaggctgaggctgcagtggccacccaggagacatccaccgtcaggctaaaggtcagctccacagccgtaaggacacagcacacaaccacccgacctgttcccgacacctcccggctgcctggggccacccctgggctcaccacggtggagatagtgacaatgtctcaccaagctctgggcgacgttgctggcagaggaaatgagaagaagcccagtagcgtgagggctctgtccattgtcctccccatcgtgctcctcgtcttcctttgcctgggggtcttccttctatggaagaactggcggcttaagaacatcaacagcatcaactttgacaaccccgtctatcagaagaccacagaggatgaggtccacatttgccacaaccaggacggctacagctacccctcgagacagatggtcagtctggaggatgacgtggcg

SEQ ID NO: 107 - RNA, 111 nt, synthetic construct
  source 1..111: mol_type = other RNA
gggaacaaaagtgcactgaaacaaaagtgcactgaaacagaggtgcactgagggaaataagagagaaaagaagagtaagaagaaatataagaccccggcgccgccaccatg

SEQ ID NO: 108 - RNA, 79 nt, synthetic construct
  source 1..79: mol_type = other RNA
gggaacaaaagtgcactgagggaaataagagagaaaagaagagtaagaagaaatataagaccccggcgccgccaccatg

SEQ ID NO: 109 - RNA, 95 nt, synthetic construct
  source 1..95: mol_type = other RNA
gggaacaaaagtgcactgaaacaaaagtgcactgagggaaataagagagaaaagaagagtaagaagaaatataagaccccggcgccgccaccatg

SEQ ID NO: 110 - RNA, 108 nt, synthetic construct
  source 1..108: mol_type = other RNA
gggaacaaagtgcactgaaacaaagtgcactgaaacagagtgcactgagggaaataagagagaaaagaagagtaagaagaaatataagaccccggcgccgccaccatg

SEQ ID NO: 111 - RNA, 105 nt, synthetic construct; identical to SEQ ID NO: 5
  source 1..105: mol_type = other RNA

SEQ ID NO: 112 - RNA, 126 nt, synthetic construct; identical to SEQ ID NO: 6
  source 1..126: mol_type = other RNA

SEQ ID NO: 113 - RNA, 130 nt, synthetic construct; identical to SEQ ID NO: 7
  source 1..130: mol_type = other RNA

SEQ ID NO: 114 - RNA, 81 nt, synthetic construct; identical to SEQ ID NO: 8
  source 1..81: mol_type = other RNA

SEQ ID NO: 115 - RNA, 83 nt, synthetic construct; identical to SEQ ID NO: 9
  source 1..83: mol_type = other RNA

SEQ ID NO: 116 - RNA, 98 nt, synthetic construct; identical to SEQ ID NO: 10
  source 1..98: mol_type = other RNA

SEQ ID NO: 117 - RNA, 103 nt, synthetic construct; identical to SEQ ID NO: 11
  source 1..103: mol_type = other RNA

SEQ ID NO: 118 - RNA, 118 nt, synthetic construct; identical to SEQ ID NO: 12
  source 1..118: mol_type = other RNA

SEQ ID NO: 119 - RNA, 114 nt, synthetic construct; identical to SEQ ID NO: 13
  source 1..114: mol_type = other RNA

SEQ ID NO: 120 - RNA, 84 nt, synthetic construct; identical to SEQ ID NO: 14
  source 1..84: mol_type = other RNA

SEQ ID NO: 121 - RNA, 111 nt, synthetic construct; identical to SEQ ID NO: 92
  source 1..111: mol_type = other RNA

SEQ ID NO: 122 - RNA, 80 nt, synthetic construct; identical to SEQ ID NO: 41
  source 1..80: mol_type = other RNA

SEQ ID NO: 123 - RNA, 113 nt, synthetic construct; identical to SEQ ID NO: 42
  source 1..113: mol_type = other RNA

SEQ ID NO: 124 - RNA, 78 nt, synthetic construct; identical to SEQ ID NO: 43
  source 1..78: mol_type = other RNA

SEQ ID NO: 125 - RNA, 113 nt, synthetic construct; identical to SEQ ID NO: 44
  source 1..113: mol_type = other RNA

SEQ ID NO: 126 - RNA, 113 nt, synthetic construct; identical to SEQ ID NO: 104
  source 1..113: mol_type = other RNA

(19) United States
(12) Patent Application Publication
(10) Pub. No.: US 2024/0200078 A1
(43) Pub. Date: Jun. 20, 2024

(54) MICRORNA-DEPENDENT MRNA SWITCHES FOR TISSUE-SPECIFIC MRNA-BASED THERAPIES

(71) Applicant: Cornell University, Ithaca, NY (US)

(72) Inventor: Seyed Hani Najafi-Shoushtari, New York, NY (US)

(21) Appl. No.: 18/056,961

(22) Filed: Nov. 18, 2022

Related U.S. Application Data
(60) Provisional application No. 63/280,882, filed on Nov. 18, 2021.

(51) Int. Cl. C12N 15/113; C12N 15/67

(52) U.S. Cl. CPC C12N 15/1138; C12N 15/67; C12N 2310/141; C12N 2310/531; C12N 2320/32

(57) ABSTRACT

Synthetic 5′UTR sequences comprising miRNA binding sites that allow for enhanced translation efficiencies of linked open reading frames, vectors having the synthetic sequences linked to an open reading frame and methods of using isolated mRNA and vectors with the synthetic sequences linked to an open reading frame, are provided.

### CROSS-REFERENCE TO RELATED APPLICATIONS

This application claims the benefit of the filing date of U.S. application Ser. No. 63/280,882 filed on Nov. 18, 2021, the disclosure of which is incorporated by reference herein,

### INCORPORATION BY REFERENCE OF SEQUENCE LISTING

A Sequence Listing is provided herewith as an xml file, “2287901.xml” created on Nov. 18, 2022, and having a size of 86,873 bytes. The content of the xml file is incorporated by reference herein in its entirety.

### BACKGROUND

Patients with heterozygous and homozygous familial hypercholesterolemia (FH) suffer in most cases from loss of function gene mutations in LDLR gene and are considerably at a high risk of developing atherosclerosis given their severely elevated serum LDL-C level. A more recent report suggests that more efficacious therapeutic approaches are needed as intense statin therapy and the recently introduced PCSK9 inhibitors are not optimal in reducing LDL-C to the level that would meet guideline recommended goals or >50%. As an alternative approach, a liver-targeted AAV-based LDLR gene replacement therapy (RGX-501, ClinicalTrials.gov NCT02651675), currently in phase 2 trial, is being considered to address the gap in treatment for patients with FH. A more recently developed small molecule inhibitor for microsomal triglyceride transfer protein (MTTP) involved in VLDL secretion pathway, for treatment of homozygous FH has shown great efficacy in lowering LDL-C but raised serious concerns with respect to long-term consequences such as fatty liver development and hepatic toxicity.

### SUMMARY

This disclosure describes isolated mRNA and constructs for, and a method for generating, functionally switchable mRNA for the development of tissue and cell-specific targeted mRNA therapies. The mRNA and constructs introduce a distal microRNA (miRNA) binding/responsive domain (miRNA binding site) as an integral part of a mRNA's 5′-untranslated region (5′-UTR) that controls its translation. Thereby, the introduced domain confers an allosteric switch mechanism that allows for translational activation to occur only in the presence of the cognate miRNA.

As described herein below, a miR-148 (a liver enriched miRNA)-dependent low density-lipoprotein receptor (LDLR) mRNA was prepared that contains an extended 5′-UTR harboring one or more miR-148 binding sites within defined secondary hairpin loop structure(s). This led to activation of the LDLR mRNA expression in a miR-148-dependent manner specifically in response to endogenous miR-148 level. miR-148 is highly expressed in hepatocytes. The miR-148a responsive LDLR mRNAs provide for RNA therapeutics for the treatment of familial and systemic hypercholesterolemia and atherosclerosis; and other settings associated with other diseases. The present disclosure thus provides synthetic messenger RNAs (mRNAs) having RNA elements including miRNA binding site(s) and optionally modified nucleotides, which allows for enhanced translational regulatory activity (activation), e.g., promoting translation of a linked open reading frame encoding a desired polypeptide. Accordingly, in one aspect the disclosure provides, mRNAs comprising a 5′ untranslated region (UTR), an initiation codon, an open reading frame encoding a polypeptide, a 3′ UTR, and at least one miRNA binding site, the presence of which provides for enhanced translational activity in cells that express the corresponding miRNA. In one embodiment, the mRNA comprises at least one other RNA element, e.g., an AU-rich RNA element or Kozak sequence. Other elements that may be included are, but are not limited to, a viral RNA element, a protein-binding RNA element, a translation initiation element, a translation enhancer element, a translation fidelity enhancing element, a mRNA nuclear export element, or a modification of base composition, or any combination thereof. Exemplary modified nucleotides include but are not limited 2-thiouridine, 2′-O-methyl-2-thiouridine, 2-selenouridine, 2′-O-methyl ribose, a modified nucleotide in which the ribose moiety is modified with an extra bridge connecting the 2′ oxygen and 4′ carbon, inosine, 2-methylguanosine, 6-methyl-adenosine, or a deoxyribonucleotide, or any combination thereof. In one embodiment, the mRNA comprises (i) a 5′ untranslated region (UTR) comprising at least one distal miRNA binding site; (ii) an open reading frame comprising an initiation codon and encoding a polypeptide; and (iii) a 3′ UTR, wherein the at least one distal miRNA binding site is located about 120-150 nucleotides, 100-120 nucleotides, 90-100 nucleotides, 80-90 nucleotides, 70-80 nucleotides, 50-60 nucleotides, 40-50 nucleotides, about 30-40 nucleotides 20-30 nucleotides, about 10-20 nucleotides, or about 6-10 nucleotides upstream of the initiation codon in the 5′ UTR. In some embodiments, the 5′ UTR is AU-rich, e.g., comprising 50% adenosine (A) or uracil (U) nucleobases. In some embodiments, the AU-rich content of the 5′UTR is >50% A and/or U nucleobases. In some embodiments, the AU-rich content if the 5′UTR is >60% A and/or U nucleobases. In some embodiments, the AU-rich content of the 5′UTR is >70% A and/or U nucleobases. In some embodiments, the AU-rich content of the 5′UTR is about 50%-55%, about 55%-60%, about 60%-65%, about 65%-70%, about 70%-75%, or about 75%-80% A and/or U nucleobases.

In one embodiment, the 5′UTR comprises (P)-(Q)-(R)-(S)-AUG, wherein P is a nucleotide sequence that may include one or more miRNA binding sites which optionally may form part of a hairpin or at least a stem of a stem loop, wherein Q is optionally present and is a nucleotide sequence that may include one or more miRNA binding sites, wherein R is optionally present and is a nucleotide sequence that may include one or more miRNA binding sites; and wherein S in a A/U rich nucleotide sequence. In one embodiment, P, Q, R and S together are up to 200 nucleotides. In one embodiment, P, Q, R and S together are up to 100 nucleotides. In one embodiment, P and R have one or more miRNA binding sites and Q does not include miRNA binding sites. In one embodiment, P, Q and R have one or more miRNA binding sites. In one embodiment, P, Q, R and S independently have 15 to 25 nucleotides, 10 to 20 nucleotides, 20 to 30 nucleotides or 30 to 50 nucleotides. In one embodiment, P, Q, R and S independently are AU rich sequences.

In some embodiments, the 5′UTR comprises an AU-rich sequence comprising a nucleotide sequence of about 5-10 nucleotides in length, about 6-10 nucleotides in length, about 7-17 nucleotides in length. In some embodiments, the AU-rich sequence is 4, 5, 6, 7, 8, 9, 10, 11, 12, 13, 14, or 15, nucleotides in length. In some embodiments, the AU-rich sequence is 5 nucleotides in length. In some embodiments, the AU-rich sequence is 6 nucleotides in length. In some embodiments, the AU-rich sequence is 7 nucleotides in length. In some embodiments, the AU-rich sequence is 8 nucleotides in length. In some embodiments, the AU-rich sequence is 9 nucleotides in length. In some embodiments, the AU-rich sequence is 10 nucleotides in length.

In one embodiment, the 5′UTR has a length of 200, 100, 50 or fewer nucleotides, has a A/U composition of 70%, 75%, 80%, 85%, 90% or 95%, has at least one miRNA binding site, e.g., near the 5′ end of the 5′UTR, and forms at least one stem-loop or hairpin structure. If there is more than one miRNA binding site, those sites may be separated by 5, 6, 7, 8, 9, 10, 15 20 or more nucleotides. The miRNA binding site may have a perfect match with the seed region of a miRNA, e.g., a seed region of 3 to 10 nucleotides, or may have mismatches in the complement of the seed region. For example, if a seed region has 9 nucleotides, the miRNA binding site may have 1, 2, 3, or 4 mismatches to that seed region. The one or more miRNA binding sites may be flanked by one or more A/U rich sequences. The miRNA binding site may form part of the at least one stem-loop or hairpin structure and in one embodiment, one strand of the stem may include from 4 to 17 nucleotides, e.g., 4 to 9 nucleotides, which nucleotides may be paired with a nucleotide in the other strand of the stem that does not result in a Watson-Crick base pair.

In one embodiment, a viral vector, e.g., an adeno-associated viral vector, or liposomes are employed to deliver DNA encoding the mRNA or the isolated mRNA, respectively. In one embodiment, the mRNA comprises two stem-loop structures and a miRNA binding site resides in the first stem-loop, e.g., primarily in the stem so that the presence of the miRNA disrupts the stem structure.

In some aspects, the disclosure provides an mRNA comprising: (i) a 5′ untranslated region (UTR) comprising at least one miRNA binding site that provides for enhanced translational activity; (ii) an open reading frame comprising an initiation codon and encoding a therapeutic or prophylactic polypeptide; and

(iii) a 3′ UTR, wherein the 5′ UTR comprises a nucleotide sequence comprising one of SEQ ID Nos. 1-18, 20-30, 40-45, 60-66, or 80-86, or a sequence with at least 80%, 82%, 84%, 86%, 88%, 90%, 92%, 94%, 95%, 96%, 98% or 99% nucleic acid sequence identity thereto. In some embodiments, the 5′UTR has sequences that form one or more stable RNA secondary structures, e.g., a hairpin or a stem-loop. In some embodiments, the stable RNA secondary structure has a deltaG of about −30 kcal/mol, about −20 to −30 kcal/mol, about −20 kcal/mol, about −10 to −20 kcal/mol, about −10 kcal/mol, about −5 to −10 kcal/mol.

In any one of the aforementioned embodiments, the 5′UTR provides for translational activation, thereby increasing or enhancing translational potency of a mRNA with the 5′UTR relative to an mRNA without the miRNA binding site, or with a different number of miRNA binding sites.

In any one of the aforementioned embodiments, the mRNA comprises a poly A tail (e.g., a poly A tail of about 100 nucleotides). In any one of the aforementioned embodiments, the mRNA comprises a 5′ cap structure.

In one embodiment, the mRNA comprises one or more of pseudouridine, N1-methylpseudouridine, 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methyluridine, 5-methyluridine, 5-methoxyuridine, and 2′-O-methyl uridine. In some embodiments, the chemical modification is selected from the group consisting of pseudouridine or a pseudouridine analog, or any combination thereof. In some embodiments, the chemical modification is N1-methylpseudouridine.

In some aspects, the disclosure provides a composition comprising the mRNA or a vector encoding the mRNA and a pharmaceutically acceptable carrier,

In some embodiments, the disclosure provides a lipid nanoparticle comprising the mRNA or vector encoding the mRNA.

In some embodiments, the disclosure provides a pharmaceutical composition comprising a lipid nanoparticle comprising the mRNA, and a pharmaceutically acceptable carrier.

In some aspects, the disclosure provides a method of increasing an amount of a polypeptide translated from an open reading frame in a mRNA, the method comprising: contacting a cell with the mRNA, or compositions or lipid nanoparticles having the mRNA.

In one embodiment, isolated mRNA is provided comprising a synthetic 5′UTR, a coding sequence for a therapeutic or prophylactic gene product, and a poly A sequence, wherein the 5′UTR comprises a nucleotide sequence that forms at least a first stem loop or hairpin structure, wherein the at least first stem or hairpin comprises a first microRNA (miRNA) binding site or a portion thereof, the presence of which in the mRNA results in enhanced translation of the gene product relative to corresponding mRNA that lacks the nucleotide sequence. In one embodiment, the 5′UTR comprises at least a second stem loop or hairpin structure which optionally comprises at least one other copy of the miRNA binding site and which optionally is 3′ to the first stem loop or hairpin structure. In one embodiment, the at least first stem or hairpin comprising the at least first miRNA binding site or the at least one other stem or hairpin includes a complementary sequence of the miRNA binding site that results in a mismatch of one or more nucleotides. In one embodiment, the mismatch includes 2, 3, 4 or 5 mismatches. In one embodiment, the 5′UTR is 200 nucleotides or less in length. In one embodiment, the 5′UTR is 100 nucleotides or less in length. In one embodiment, the nucleotide sequence comprising the at least first miRNA binding site is in the first 25% of the 5′UTR. In one embodiment, the nucleotide sequence comprising the at least first miRNA binding site is in the first half of the 5′UTR. In one embodiment, the miRNA is expressed in a tissue- or cell- specific manner. In one embodiment, the 5′UTR further comprises a second nucleotide sequence that forms a stem loop structure and is 3′ to the nucleotide sequence comprising the at least first miRNA binding site. In one embodiment, the second nucleotide sequence comprises a further miRNA binding site which optionally is for the same miRNA as the first miRNA binding site. In one embodiment, the 5′UTR has a G-C poor sequence. In one embodiment, the 5′UTR has less than 50%, 40%, 30%, 20% or 10% G-C content. In one embodiment, the isolated mRNA comprises three or fewer miRNA binding sites. In one embodiment, the 5′UTR comprises an A/U rich sequence. In one embodiment, the 5′UTR comprises one of SEQ ID Nos. 1-18, 20-30, 40-46, 60-66 or 80-86, or a sequence with at least 80%, 82%, 84%, 86%, 88%, 90%, 92%, 94%, 95%, 96%, 98% or 99% nucleic acid sequence identity thereto. In one embodiment, at least one microRNA binding site is not part of a stem loop or hairpin. In one embodiment, the microRNA binding site is 30, 25, 20, 15, 14, 13, 12, 11, 10, 9, 8, 7, 6, or 5 nucleotides in length. In one embodiment, the microRNA binding site is 11, 10, 9, 8, 7, 6, or 5 nucleotides in length.

Further provided is a vector, e.g., a DNA vector, comprising a nucleic acid sequence encoding the mRNA. In one embodiment, the vector is a plasmid. In one embodiment, the vector is a viral vector.

Also provided is a composition comprising the isolated mRNA or the vector. In one embodiment, the composition comprises liposomes or nanoparticles.

In one embodiment, a method is provided to alter expression of a gene product in a mammalian cell, comprising: contacting the cell with an effective amount of the isolated mRNA or the vector. In one embodiment, the mammal is a human. In one embodiment, the cell is in a mammal. In one embodiment,

the composition comprises liposomes. In one embodiment, the liposomes comprise or more of DC-cholesterol, 1,2-dioleoyl-sn-glycero-3-phosphoethanolamine (DOPE), an ionizable cationic lipid or a lipidoid. In one embodiment, the composition comprises nanoparticles. In one embodiment, the composition is systemically administered. In one embodiment, the composition is orally administered. In one embodiment, the composition is injected. In one embodiment, the composition comprises about 0.01 mg/kg to about 100 mg/kg, about 0.05 mg/kg to about 10 mg/kg, about 10 mg/kg to about 75 mg/kg or
about 1 mg/kg to about 100 mg/kg of the mRNA or the vector. The presence of the miRNA binding sites can reduce the inhibitory effect of the corresponding.miRNA.

### BRIEF DESCRIPTION OF FIGURES

FIGS. 1A-B. Oligonucleotide mediated miR-148a antagonism elevates LDLR and ABAC1 expression in primary human hepatocytes, a mechanism that contributes reduced LDL and elevated HDL levels. A) Schematic presentation, (B) In vitro cell expression analysis.

FIG. 2. The 7 methylguanosine (m7G) 5′ cap structure (circle) at the 5′ end of the mRNA and the poly(A) tail (An) at the 3′ end stabilize the mRNA and stimulate translation. The 5′ untranslated region (UTR) contains secondary and tertiary structures and other sequence elements. RNA structures such as pseudoknots, hairpins and RNA Gquadruplexes (RG4s), as well as upstream open reading frames (uORFs) and upstream start codons (uAUGs), mainly inhibit translation. Internal ribosomal entry sites (IRESs) mediate translation initiation independently of the cap. RNA modifications, or RNA binding proteins (RBPs) and long non-coding RNAs (IncRNAs) that interact with RNA binding sites or form ribonucleoprotein (RNP) complexes, as well as the Kozak sequence around the start codon, can additionally regulate translation initiation.

FIG. 3. Secondary structure of the generic LDLR mRNA 5′UTR as predicted by m-fold program.

FIG. 4. Schematic representation of proposed mechanism of microRNAeffector regulated of mRNA constructs.

FIGS. 5A-5D. Secondary structures prediction of the engineered miR-148a effector regulated 5′UTRs used for C1, C2, C3, and C4 (SEQ ID NOs:87-90).

FIG. 6. Secondary structures prediction of the miR-148a effector regulated 5′UTRs of C6 construct with multiple 8 and 9-mer binding sites and a relatively GC-rich M domain (SEQ IDNO:91).

FIG. 7. Protein expression of LDLR mRNAs Constructs (C1-C9) in HepG2 at indicated times after transfection as compared to unmodified control construct (Ctrl). Immunoblot analysis for LDLR and ABCA1 are shown. Actin is used as an internal loading control. UT: Untreated, FDS: flanking dinucleotide sequence (#1: AAAAG/AUUU; SEQ ID NO:20) #2: AAUAAA; SEQ ID NO:21).

FIG. 8. Protein expression of unmodified control (Ctrl) and miR-148a binding C2 LDLR mRNAs constructs in HepG2 cells after 24h as compared to endogenous LDLR expression in response to Anti-LNA mediated miR-148a depletion.

FIG. 9. Injection of mRNA constructs into male LDLR KO mice fed a western-type of diet after four days. Levels and distribution of cholesterol isolated by Fast Protein Liquid Chromatography (FPLC) fractionation from pooled sera of LDLR. KO mice (n=5). Total cholesterol concentration from pooled sera is depicted in the table.

FIG. 10. Hepatic ABCA1 protein expression analysis from male LDLR KO mice treated with C2 and C3 mRNA constructs compared to generic LDLR mRNA treatment.

FIG. 11. Injection of mRNA constructs into female LDLR KO mice fed a western-type of diet after four days. Levels and distribution of cholesterol isolated by Fast Protein Liquid

Chromatography (FPLC) fractionation from pooled sera of LDLR. KO mice (n=5). Total cholesterol ) concentration from pooled sera are shown in the table.

FIG. 12. MIR-33a-3p improves the expression of synthetic WT-LDLR mRNA construct as well as elevates ABCA1 expression.

FIG. 13. Protein expression of LDLR mRNAs Constructs in HepG2 at indicated times after transfection as compared to endogenous LDLR level and unmodified control construct (Ctrl). Immunoblot analysis for LDLR are shown. Actin is used as an internal loading control. UT: Untreated, FDS: flanking dinucleotide sequence (#1: AAAAG/AUUU (SEQ ID NO:22), #2: AAUAAA (SEQ ID NO:23), #3: AACAAA (SEQ ID NO:24).

FIGS. 14A-14F. Additional secondary structures (SEQ ID NOS:92-103).

### DETAILED DESCRIPTION

### Definitions

The terms “treat” and “treating” as used herein refer to (i) preventing a pathologic condition from occurring (e.g., prophylaxis); (ii) inhibiting the pathologic condition or arresting its development; (iii) relieving the pathologic condition; and/or (iv) ameliorating, alleviating, lessening, and removing symptoms of a condition. A compound, e.g., nucleic acid molecule, described herein may be in an amount in a formulation or medicament, which is an amount that can lead to a biological effect, or lead to ameliorating, alleviating, lessening, relieving, diminishing or removing symptoms of a condition, e.g., disease, for example.

The term “therapeutically effective amount” as used herein refers to an amount of a compound, or an amount of a combination of compounds, to treat, inhibit or prevent a disease or disorder, or to prevent, inhibit or treat a symptom of the disease or disorder, in a subject.

The terms “subject,” “patient” or “subject in need thereof” refers to a living organism suffering from or prone to a disease or condition that can be treated by administration of a compound, pharmaceutical composition, or mixture. Non-limiting examples include humans, other mammals, bovines, rats, mice, dogs, monkeys, goat, sheep, cows, deer, and other non-mammalian animals. In some embodiments, a patient is human. In some embodiments, a patient is a domesticated animal. In some embodiments, a patient is a dog. In some embodiments, a patient is livestock animal. In some embodiments, a patient is a mammal. In some embodiments, a patient is a cat. In some embodiments, a patient is a horse. In some embodiments, a patient is bovine. In some embodiments, a patient is a canine. In some embodiments, a patient is a feline. In some embodiments, a patient is a non-human primate. In some embodiments, a patient is a mouse. In some embodiments, a patient is a rat. In some embodiments, a patient is a newborn animal. In some embodiments, a patient is a newborn human. In some embodiments, a patient is a newborn mammal. In some embodiments, a patient is an elderly animal. In some embodiments, a patient is an elderly human. In some embodiments, a patient is an elderly mammal. In some embodiments, a patient is a geriatric patient.

As used herein, the term “isolated” in the context of nucleic acid molecule refers to a nucleic acid molecule which is separated from other molecules which are present in the natural source of the nucleic acid molecule.

As used herein, the terms “prevent”, “prevention” and “preventing” refer to obtaining a prophylactic benefit in a subject receiving a pharmaceutical composition. With respect to achieving a prophylactic benefit, the object is to delay or prevent the symptoms associated with the pathological condition or disorder. A “prophylactically effective amount” refers to that amount of a prophylactic agent, sufficient to achieve at least one prophylactic benefit in a subject receiving the composition.

By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine, and uracil).

Polynucleotide modifications, e.g., for protecting exogenous polynucleotides from degradation, include, but are not limited to, addition of terminal amino group(s) and the use of modified internucleotide linkages such as, for example, phosphorothioates, phosphoramidates, and O-methyl ribose or deoxyribose residues,

Exemplary nucleic acid analogs may have a modified pyrimidine nucleobase, or a purine or pyrimidine base that contains an exocyclic amine.

Other nucleotide modifications include peptide nucleic acid (PNA) or locked nucleic acid (LNA), analogs of methyleneoxy (4′-CH₂-O-2′) BNA, phosphorothioate-methyleneoxy (4′-CH₂-0-2′) BNA and 2′-thio-BNAs, have also been prepared (Kumar et al., 1998), as well as amino- and 2′-methylamino-BNA. Preparation of locked nucleoside analogs comprising oligodeoxyribonucleotide duplexes as substrates for nucleic acid polymerases has also been described (WO 99/14226). Furthermore, synthesis of 2′-amino-BNA, a conformationally restricted high-affinity oligonucleotide analog has been described in the art (Singh et al., 1998).

Modified sugar moieties may be used, e.g., to alter, typically increase, the affinity of the polynucleotide for its target and/or increase nuclease resistance. A representative list of modified sugars includes but is not limited to bicyclic modified sugars (BNA's), including methyleneoxy (4′-CH₂-0-2′) BNA and ethyleneoxy (4′-(CH₂)2-O-2′ bridge) BNA ; substituted sugars, especially 2′-substituted sugars having a 2′-F, 2′-OCH, or a 2′-O(CH₂)2-OCH3 substituent group; and 4′-thio modified sugars. Sugars can also be replaced with sugar mimetic groups among others. Methods for the preparations of modified sugars are well known to those skilled in the art. Some representative patents and publications that teach the preparation of such modified sugars include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; 5,700,920; 6,531,584; and 6,600,032; and WO 2005/121371.

As used herein, the terms “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

As used herein, the term “contacting” means establishing a physical connection between two or more entities. For example, contacting a cell with an mRNA or a lipid nanoparticle composition means that the cell and mRNA or lipid nanoparticle are made to share a physical connection. Methods of contacting cells with external entities both in vivo, in vitro, and ex vivo are well known in the biological arts. In exemplary embodiments of the disclosure, the step of contacting a mammalian cell with a composition (e.g., an isolated mRNA, nanoparticle, or pharmaceutical composition of the disclosure) is performed in vivo. For example, contacting a lipid nanoparticle composition and a cell (for example, a mammalian cell) which may be disposed within an organism (e.g., a mammal) may be performed by any suitable administration route (e.g., parenteral administration to the organism, including intravenous, intramuscular, intradermal, and subcutaneous administration). For a cell present in vitro, a composition (e.g., a lipid nanoparticle or an isolated mRNA) and a cell may be contacted, for example, by adding the composition to the culture medium of the cell and may involve or result in transfection. Moreover, more than one cell may be contacted by a nanoparticle composition.

As used herein, the term “denaturation” refers to the process by which the hydrogen bonding between base paired nucleotides in a nucleic acid is disrupted, resulting in the loss of secondary and/or tertiary nucleic acid structure (e.g., the separation of previously annealed strands). Denaturation can occur by the application of an external substance, energy, or biochemical process to a nucleic acid. For example, local denaturation of nucleic acid structure by enzymatic activity occurs when biologically important transactions such as DNA replication, transcription, translation, or DNA repair need to occur. Folded structures (e.g., secondary and tertiary nucleic acid structures) of an mRNA can constitute a barrier to the scanning function of the PIC or the elongation function of the ribosome, resulting in a lower translation rate. During translation initiation, helicase activity provided by elFs (e.g., elF4A) can denature or unwind duplexed, double-stranded RNA structure.

As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; and (4) post-translational modification of a polypeptide or protein,

As used herein, the term “identity” refers to the overall relatedness between polymeric molecules, e.g., between polynucleotide molecules (e.g., DNA molecules and/or RNA molecules) and/or between polypeptide molecules. Calculation of the percent identity of two polynucleotide sequences, for example, can be performed by aligning the two sequences for optimal comparison purposes (e.g., gaps can be introduced in one or both of a first and a second nucleic acid sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap which needs to be introduced for optimal alignment of the two sequences. The comparison of sequences and determination of percent identity between two sequences can be accomplished using a mathematical algorithm. For example, the percent identity between two nucleotide sequences can be determined using methods such as those described in Computational Molecular Biology, Lesk, A. M., ed., Oxford University Press, New York, 1988; Biocomputing: Informatics and Genome Projects, Smith, D. W., ed., Academic Press, New York, 1993; Sequence Analysis in Molecular Biology, von Heinje, G., Academic Press, 1987; Computer Analysis of Sequence Data, Part I, Griffin, A. M., and Griffin, H. G., eds., Humana Press, New Jersey, 1994; and Sequence Analysis Primer, Gribskov, M. and Devereux, J., eds., M Stockton Press, New York, 1991; each of which is incorporated herein by reference. For example, the percent identity between two nucleotide sequences can be determined using the algorithm of Meyers and Miller (CABIOS, 1989, 4:11-17), which has been incorporated into the ALIGN program (version 2.0) using a PAM120 weight residue table, a gap length penalty of 12 and a gap penalty of 4. The percent identity between two nucleotide sequences can, alternatively, be determined using the GAP program in the GCG software package using an NWSgapdna.CMP matrix. Methods commonly employed to determine percent identity between sequences include, but are not limited to those disclosed in Carillo, H., and Lipman, D., SIAM J Applied Math., 48:1073 (1988); incorporated herein by reference. Techniques for determining identity are codified in publicly available computer programs. Exemplary computer software to determine homology between two sequences include, but are not limited to, GCG program package, Devereux et al., Nucleic Acids Research, 12(1): 387,1984, BLASTP, BLASTN, and FASTA, Altschul, S. F. et al., J. Molec. Biol., 215, 403, 1990.

A “fragment,” as used herein, refers to a portion. For example, fragments of proteins may include polypeptides obtained by digesting full-length protein isolated from cultured cells or obtained through recombinant DNA techniques.

As used herein, the term “AU-rich” refers to the nucleobase composition of a polynucleotide (e.g., mRNA), or any portion thereof (e.g., an RNA element), comprising adenine (A) and/or uracil (U) nucleobases, or derivatives or analogs thereof, wherein the AU-content is greater than 50%. The term “AU-rich” may be in reference to a 5′ UTR or any discrete sequence, fragment, or segment thereof which comprises greater than 50% or more AU-content. In some embodiments of the disclosure, AU-rich polynucleotides, or any portions thereof, are exclusively comprised of A and/or U nucleobases.

As used herein, the term “AU-content” refers to the percentage of nucleobases in a polynucleotide (e.g., mRNA), or a portion thereof (e.g., an RNA element), that are either A and U nucleobases, or derivatives or analogs thereof, (from a total number of possible nucleobases, including A and thymine (T) or U), and derivatives or analogs thereof, in DNA and in RNA. The term “AU-content” may refer to a 5′UTR, or any discrete sequence, fragment, or segment thereof.

As used herein, “heterologous” indicates that a sequence (e.g., an amino acid sequence or the polynucleotide that encodes an amino acid sequence) is not normally present in a given natural polypeptide or polynucleotide. For example, an amino acid sequence that corresponds to a domain or motif of one protein may be heterologous to a second protein.

As used herein, the term “hybridization” refers to the process of a first single-stranded nucleic acid, or a portion, fragment, or region thereof, annealing to a second single-stranded nucleic acid, or a portion, fragment, or region thereof, either from the same or separate nucleic acid molecules, mediated by Watson-Crick base pairing to form a secondary and/or tertiary structure. Complementary strands of linked nucleobases able to undergo hybridization can be from either the same or separate nucleic acids. Due to the thermodynamically favorable hydrogen bonding interaction between complementary base pairs, hybridization is a fundamental property of complementary nucleic acid sequences. Such hybridization of nucleic acids, or a portion or fragment thereof, may occur with “near” or “substantial” complementarity, as well as with exact complementarity.

As used herein, an “insertion” or an “addition” refers to a change in an amino acid or nucleotide sequence resulting in the addition of one or more amino acid residues or nucleotides, respectively, to a molecule as compared to a reference sequence, for example, the sequence found in a naturally-occurring molecule.

As used herein, an “insertion site” is a position or region of a scaffold polypeptide that is amenable to insertion of an amino acid sequence of a heterologous polypeptide. It is to be understood that an insertion site also may refer to the position or region of the polynucleotide that encodes the polypeptide (e.g., a codon of a polynucleotide that codes for a given amino acid in the scaffold polypeptide). In some embodiments, insertion of an amino acid sequence of a heterologous polypeptide into a scaffold polypeptide has little to no effect on the stability (e.g., conformational stability), expression level, or overall secondary structure of the scaffold polypeptide.

As used herein, the term “isolated” refers to a substance or entity that has been separated from at least some of the components with which it was associated (whether in nature or in an experimental setting). Isolated substances may have varying levels of purity in reference to the substances from which they have been associated. Isolated substances and/or entities may be separated from at least about 10%, about 20%, about 30%, about 40%, about 50%, about 60%, about 70%, about 80%, about 90%, or more of the other components with which they were initially associated. In some embodiments, isolated agents are more than about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components.

As used herein, an “mRNA” refers to a messenger ribonucleic acid. An mRNA may be naturally or non-naturally occurring or synthetic. For example, an mRNA may include modified and/or non-naturally occurring components such as one or more nucleobases, nucleosides, nucleotides, or linkers. An mRNA may include a cap structure, a 5′ transcript leader, a 5′ untranslated region, an initiator codon, an open reading frame, a stop codon, a chain terminating nucleoside, a stem-loop, a hairpin, a polyA sequence, a polyadenylation signal, and/or one or more cis-regulatory elements. An mRNA may have a nucleotide sequence encoding a polypeptide. Translation of an mRNA, for example, in vivo translation of an mRNA inside a mammalian cell, may produce a polypeptide. Traditionally, the basic components of a natural mRNA molecule include at least a coding region, a 5′-untranslated region (5′-UTR), a 3′UTR, a 5′ cap and a polyA sequence.

As used herein “modified” or “modification” refers to a changed state or a change in composition or structure of a polynucleotide (e.g., mRNA), Polynucleotides may be modified in various ways including chemically, structurally, and/or functionally. For example, polynucleotides may be structurally modified by the incorporation of one or more RNA elements, wherein the RNA element comprises a sequence and/or an RNA secondary structure(s) that provides one or more functions (e.g., translational regulatory activity). Accordingly, polynucleotides of the disclosure may be comprised of one or more modifications (e.g., may include one or more chemical, structural, or functional modifications, including any combination thereof).

As used herein, the term “nucleobase” (alternatively “nucleotide base” or “nitrogenous base”) refers to a purine or pyrimidine heterocyclic compound found in nucleic acids, including any derivatives or analogs of the naturally occurring purines and pyrimidines that confer improved properties (e.g., binding affinity, nuclease resistance, chemical stability) to a nucleic acid or a portion or segment thereof. Adenine, cytosine, guanine, thymine, and uracil are the nucleobases predominately found in natural nucleic acids. Other natural, non-natural, and/or synthetic nucleobases, as known in the art and/or described herein, can be incorporated into nucleic acids.

As used herein, the term “nucleic acid” is used in its broadest sense and encompasses any compound and/or substance that includes a polymer of nucleotides, or derivatives or analogs thereof. These polymers are often referred to as “polynucleotides”. Accordingly, as used herein the terms “nucleic acid” and “polynucleotide” are equivalent and are used interchangeably. Exemplary nucleic acids or polynucleotides of the disclosure include, but are not limited to, ribonucleic acids (RNAs), deoxyribonucleic acids (DNAs), DNA-RNA hybrids, RNAi-inducing agents, RNAi agents, siRNAs, shRNAs, mRNAs, modified mRNAs, miRNAs, antisense RNAs, ribozymes, catalytic DNA, RNAs that induce triple helix formation, threose nucleic acids (TNAs), glycol nucleic acids (GNAs), peptide nucleic acids (PNAs), locked nucleic acids (LNAs, including LNA having a β-D-ribo configuration, α-LNA having an α-L-ribo configuration (a diastereomer of LNA), 2′-amino-LNA having a 2′-amino functionalization, and 2′-amino-α-LNA having a 2′-amino functionalization) or hybrids thereof.

As used herein, the term “polypeptide” or “polypeptide of interest” refers to a polymer of amino acid residues typically joined by peptide bonds that can be produced naturally (e.g., isolated or purified) or synthetically.

As used herein, the term “stable RNA secondary structure” refers to a structure, fold, or conformation adopted by an RNA molecule, or local segment or portion thereof, that is persistently maintained under physiological conditions and characterized by a low free energy state. Typical examples of stable RNA secondary structures include duplexes, hairpins, and stem-loops. Stable RNA secondary structures are known in the art to exhibit various biological activities.

As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

As used herein, the terms “transfect”, “transfection” or “transfecting” refer to the act or method of introducing a molecule, usually a nucleic acid, into a cell.

### mRNA Constructs

The present disclosure provides synthetic polynucleotides (e.g., mRNAs or vectors encoding mRNAs) comprising a modification (e.g., an RNA element), wherein the modification provides a desired translational regulatory activity. In some embodiments, the disclosure provides a polynucleotide comprising a 5′ untranslated region (UTR), an initiation codon, an open reading frame encoding a polypeptide, a 3′ UTR, and at least one modification, wherein the at least one modification provides for enhanced translational activity. In some embodiments, the desired translational regulatory activity is an increase in the amount of polypeptide translated from the full open reading frame.

Accordingly, the present disclosure provides a polynucleotide, e.g., an mRNA, comprising an RNA element that comprises a sequence and/or an RNA secondary structure(s) that provides a desired translational activity as described herein.

In some embodiments, the disclosure provides mRNAs comprising RNA elements in the 5′UTR comprising one or more miRNA binding sites. In some embodiments, the disclosure provides mRNAs comprising RNA elements in the 5′UTR comprising AU-rich sequences. An RNA element is a portion, fragment or segment of an RNA molecule that has biological significance. In some embodiments, an RNA element comprises a stable RNA secondary structure, e.g., a stem loop or hairpin having sequences forming the base paired region that include one or more mismatches. For example, the 5′UTR may include a first miRNA binding site and another miRNA binding site with or without perfect complementarity to the first miRNA binding site.

Typical examples of stable RNA secondary structures include duplexes, hairpins, and stem-loops.

### mRNA CONSTRUCT COMPONENTS

An mRNA may be a naturally or non-naturally occurring mRNA. An mRNA may include one or more modified nucleobases, nucleosides, or nucleotides, as described below, in which case it may be referred to as a “modified mRNA”. As described herein “nucleoside” is defined as a compound containing a sugar molecule (e.g., a pentose or ribose) or derivative thereof in combination with an organic base (e.g., a purine or pyrimidine) or a derivative thereof (also referred to herein as “nucleobase”). As described herein, “nucleotide” is defined as a nucleoside including a phosphate group.

An mRNA may include a 5′ untranslated region (5′-UTR), a 3′ untranslated region (3′-UTR), and/or a coding region (e.g., an open reading frame). An exemplary 5′ UTR for use in the constructs is shown in SEQ ID Nos: 1-14 or 40-45, or a sequence with at least 80%, 82%, 84%, 86%, 88%, 90%, 92%, 94%, 95%, 96%, 98% or 99% nucleic acid sequence identity thereto. An mRNA may include any suitable number of base pairs, including tens (e.g., 10, 20, 30, 40, 50, 60, 70, 80, 90 or 100), hundreds (e.g., 200, 300, 400, 500, 600, 700, 800, or 900) or thousands (e.g., 1000, 2000, 3000, 4000, 5000, 6000, 7000, 8000, 9000, 10,000) of base pairs. Any number (e.g., all, some, or none) of nucleobases, nucleosides, or nucleotides may be an analog of a canonical species, substituted, modified, or otherwise non-naturally occurring. In certain embodiments, all of a particular nucleobase type may be modified.

In some embodiments, an mRNA as described herein may include a 5′ cap structure, a chain terminating nucleotide, optionally a Kozak sequence (also known as a Kozak consensus sequence), a stem loop, a polyA sequence, and/or a polyadenylation signal.

A 5′ cap structure or cap species is a compound including two nucleoside moieties joined by a linker and may be selected from a naturally occurring cap, a non-naturally occurring cap or cap analog, or an anti-reverse cap analog (ARCA). A cap species may include one or more modified nucleosides and/or linker moieties. For example, a natural mRNA cap may include a guanine nucleotide and a guanine (G) nucleotide methylated at the 7 position joined by a triphosphate linkage at their 5′ positions, e.g., m⁷G(5′)ppp(5′)G, commonly written as m⁷GpppG. A cap species may also be an anti-reverse cap analog. A non-limiting list of possible cap species includes m⁷GpppG, m⁷Gpppm⁷G, m⁷3′dGpppG, m₂⁷,O3′GpppG, m₂⁷,O3′GppppG, m₂⁷,O2′GppppG, m⁷Gpppm⁷G, m⁷3′dGpppG, m₂⁷,O3′GpppG, m₂⁷,O3′GppppG, and m₂⁷,O2′GppppG.

An mRNA may instead or additionally include a stem loop. A stem loop may include 2, 3, 4, 5, 6, 7, 8, or more nucleotide base pairs. For example, a stem loop may include 4, 5, 6, 7, or 8 nucleotide base pairs. A stem loop may be located in any region of an mRNA. For example, a stem loop may be located in, before, or after an untranslated region (a 5′ untranslated region or a 3′ untranslated region), a coding region, or a polyA sequence or tail.

An mRNA may instead or additionally include a polyA sequence and/or polyadenylation signal. A polyA sequence may be comprised entirely or mostly of adenine nucleotides or analogs or derivatives thereof. A polyA sequence may be a tail located adjacent to a 3′ untranslated region of an mRNA. In some embodiments, a polyA sequence may affect the nuclear export, translation, and/or stability of an mRNA. An mRNA may include different microRNA binding sites, e.g., of different sequence composition or length but specific for the same miRNA or for different miRNAs.

### Regions Having a 5′ Cap

The polynucleotide comprising an mRNA encoding a polypeptide of the present disclosure can further comprise a 5′ cap. The 5′ cap useful for polypeptide encoding mRNA can bind the mRNA Cap Binding Protein (CBP), thereby increasing mRNA stability. The cap can further assist the removal of 5′ proximal introns removal during mRNA splicing.

In some embodiments, the polynucleotide comprising an mRNA encoding a polypeptide of the present disclosure comprises a non-hydrolyzable cap structure preventing decapping and thus increasing mRNA half-life. Because cap structure hydrolysis requires cleavage of 5′-ppp-5′ phosphorodiester linkages, modified nucleotides can be used during the capping reaction. For example, a Vaccinia Capping Enzyme from New England Biolabs (Ipswich, Mass.) can be used with a-thio-guanosine nucleotides according to the manufacturer's instructions to create a phosphorothioate linkage in the 5′-ppp-5′ cap. Additional modified guanosine nucleotides can be used such as a-methyl-phosphonate and seleno-phosphate nucleotides.

In certain embodiments, the 5′ cap comprises 2′0-methylation of the ribose sugars of 5′-terminal and/or 5′-anteterminal nucleotides on the 2′-hydroxyl group of the sugar ring. In other embodiments, the caps for the polypeptide-encoding mRNA include cap analogs, which herein are also referred to as synthetic cap analogs, chemical caps, chemical cap analogs, or structural or functional cap analogs, differ from natural (i.e., endogenous, wild-type or physiological) 5′-caps in their chemical structure, while retaining cap function. Cap analogs can be chemically (i.e., non-enzymatically) or enzymatically synthesized and/or linked to the polynucleotides of the disclosure,

For example, the Anti-Reverse Cap Analog (ARCA) cap contains two guanines linked by a 5′-5′-triphosphate group, wherein one guanine contains an N7 methyl group as well as a 3′-O-methyl group (i.e., N7,3′-O-dimethyl-guanosine-5′-triphosphate-5′-guanosine (m.sup.7G-3′mppp-G; which can equivalently be designated 3′ O-Me-m⁷G(5′)ppp(5′)G). The 3′-O atom of the other, unmodified, guanine becomes linked to the 5′-terminal nucleotide of the capped polynucleotide. The N7- and 3′-O-methlyated guanine provides the terminal moiety of the capped polynucleotide. Another exemplary cap is mCAP, which is similar to ARCA but has a 2′-O-methyl group on guanosine (i.e., N7,2′-O-dimethyl-guanosine-5′-triphosphate-5′-guanosine, m⁷Gm-ppp-G). In some embodiments, the cap is a dinucleotide cap analog. As a non-limiting example, the dinucleotide cap analog can be modified at different phosphate positions with a boranophosphate group or a phophoroselenoate group such as the dinucleotide cap analogs described in U.S. Patent No. 8,519,110.

In another embodiment, the cap is a cap analog is a N7-(4-chlorophenoxyethyl) substituted dinucleotide form of a cap analog known in the art and/or described herein. Non-limiting examples of a N7-(4-chlorophenoxyethyl) substituted dinucleotide form of a cap analog include a N7-(4-chlorophenoxyethyl)-G(5′)ppp(5′)G and a N7-(4-chlorophenoxyethyl)-m³′-OG(5′)ppp(5′)G cap analog. See, e.g., the various cap analogs and the methods of synthesizing cap analogs described in Kore et al. (2013) Bioorganic & Medicinal Chemistry 21:4570-4574. In another embodiment, a cap analog of the present disclosure is a 4-chloro/bromophenoxyethyl analog.

While cap analogs allow for the concomitant capping of a polynucleotide or a region thereof, in an in vitro transcription reaction, up to 20% of transcripts can remain uncapped. This, as well as the structural differences of a cap analog from an endogenous 5′-cap structures of nucleic acids produced by the endogenous, cellular transcription machinery, can lead to reduced translational competency and reduced cellular stability.

An mRNA of the present disclosure can also be capped post-manufacture (whether IVT or chemical synthesis), using enzymes, in order to generate more authentic 5′-cap structures. As used herein, the phrase “more authentic” refers to a feature that closely mirrors or mimics, either structurally or functionally, an endogenous or wild type feature. That is, a “more authentic” feature is better representative of an endogenous, wild-type, natural or physiological cellular function and/or structure as compared to synthetic features or analogs, etc., of the prior art, or which outperforms the corresponding endogenous, wild-type, natural or physiological feature in one or more respects.

Non-limiting examples of more authentic 5′ cap structures of the present disclosure are those which, among other things, have enhanced binding of cap binding proteins, increased half-life, reduced susceptibility to 5′ endonucleases and/or reduced 5′decapping, as compared to synthetic 5′cap structures known in the art (or to a wild-type, natural or physiological 5′cap structure). For example, recombinant Vaccinia Virus Capping Enzyme and recombinant 2′-0-methyltransferase enzyme can create a canonical 5′-5′-triphosphate linkage between the 5′-terminal nucleotide of a polynucleotide and a guanine cap nucleotide wherein the cap guanine contains an N7 methylation and the 5′-terminal nucleotide of the mRNA contains a 2′-O-methyl. Such a structure is termed the Cap 1 structure. This cap results in a higher translational-competency and cellular stability and a reduced activation of cellular pro-inflammatory cytokines, as compared, e.g., to other 5′cap analog structures known in the art. Cap structures include, but are not limited to, 7mG(5′)ppp(5′)N,pN2p (cap 0), 7mG(5′)ppp(5′)N1mpNp (cap 1), and 7mG(5′)-ppp(5′)N1mpN2mp (cap 2).

According to the present disclosure, 5′ terminal caps can include endogenous caps or cap analogs. According to the present disclosure, a 5′ terminal cap can comprise a guanine analog. Useful guanine analogs include, but are not limited to, inosine, N1-methyl-guanosine, 2′fluoro-guanosine, 7-deaza-guanosine, 8-oxo-guanosine, 2-amino-guanosine, LNA-guanosine, and 2-azido-guanosine.

### Poly-A Tails

In some embodiments, a polynucleotide comprising an mRNA encoding a polypeptide of the present disclosure further comprises a poly A tail. In further embodiments, terminal groups on the poly-A tail can be incorporated for stabilization. In other embodiments, a poly-A tail comprises des-3′ hydroxyl tails. The useful poly-A tails can also include structural moieties or 2′-Omethyl modifications as taught by Li et al. (2005) Current Biology 15:1501-1507.

In one embodiment, the length of a poly-A tail, when present, is greater than 30 nucleotides in length. In another embodiment, the poly-A tail is greater than 35 nucleotides in length (e.g., at least or greater than about 35, 40, 45, 50, 55, 60, 70, 80, 90, 100, 120, 140, 160, 180, 200, 250, 300, 350, 400, 450, 500, 600, 700, 800, 900, 1,000, 1,100, 1,200, 1,300, 1,400, 1,500, 1,600, 1,700, 1,800, 1,900, 2,000, 2,500, and 3,000 nucleotides).

In some embodiments, the polynucleotide or region thereof includes from about 30 to about 3,000 nucleotides (e.g., from 30 to 50, from 30 to 100, from 30 to 250, from 30 to 500, from 30 to 750, from 30 to 1,000, from 30 to 1,500, from 30 to 2,000, from 30 to 2,500, from 50 to 100, from 50 to 250, from 50 to 500, from 50 to 750, from 50 to 1,000, from 50 to 1,500, from 50 to 2,000, from 50 to 2,500, from 50 to 3,000, from 100 to 500, from 100 to 750, from 100 to 1,000, from 100 to 1,500, from 100 to 2,000, from 100 to 2,500, from 100 to 3,000, from 500 to 750, from 500 to 1,000, from 500 to 1,500, from 500 to 2,000, from 500 to 2,500, from 500 to 3,000, from 1,000 to 1,500, from 1,000 to 2,000, from 1,000 to 2,500, from 1,000 to 3,000, from 1,500 to 2,000, from 1,500 to 2,500, from 1,500 to 3,000, from 2,000 to 3,000, from 2,000 to 2,500, and from 2,500 to 3,000).

In some embodiments, the poly-A tail is designed relative to the length of the overall polynucleotide or the length of a particular region of the polynucleotide. This design can be based on the length of a coding region, the length of a particular feature or region or based on the length of the ultimate product expressed from the polynucleotides,

In this context, the poly-A tail can be 10, 20, 30, 40, 50, 60, 70, 80, 90, or 100% greater in length than the polynucleotide or feature thereof. The poly-A tail can also be designed as a fraction of the polynucleotides to which it belongs. In this context, the poly-A tail can be 10, 20, 30, 40, 50, 60, 70, 80, or 90% or more of the total length of the construct, a construct region or the total length of the construct minus the poly-A tail. Further, engineered binding sites and conjugation of polynucleotides for Poly-A binding protein can enhance expression.

Additionally, multiple distinct polynucleotides can be linked together via the PABP (Poly-A binding protein) through the 3′-end using modified nucleotides at the 3′-terminus of the poly-A tail. Transfection experiments can be conducted in relevant cell lines at and protein production can be assayed by ELISA at 12 hours, 24 hours, 48 hours, 72 hours and day 7 post-transfection.

In some embodiments, the polynucleotides of the present disclosure are designed to include a polyA-G Quartet region. The G-quartet is a cyclic hydrogen bonded array of four guanine nucleotides that can be formed by G-rich sequences in both DNA and RNA. In this embodiment, the G-quartet is incorporated at the end of the poly-A tail. The resultant polynucleotide is assayed for stability, protein production and other parameters including half-life at various time points. It has been discovered that the polyA-G quartet results in protein production from an mRNA equivalent to at least 75% of that seen using a poly-A tail of 120 nucleotides alone.

### Modified mRNAs

In some embodiments, an mRNA of the disclosure comprises one or more modified nucleobases, nucleosides, or nucleotides. In some embodiments, modified mRNAs may have useful properties, including enhanced stability, intracellular retention, enhanced translation, and/or the lack of a substantial induction of the innate immune response of a cell into which the mRNA is introduced, as compared to a reference unmodified mRNA. Therefore, use of modified mRNAs may enhance the efficiency of protein production, intracellular retention of nucleic acids, as well as possess reduced immunogenicity.

Accordingly, in some embodiments, an mRNA described herein comprises a modification, wherein the modification is the incorporation of one or more chemically modified nucleotides. In some embodiments, one or more chemically modified nucleotides is incorporated into the initiation codon and functions to increases binding affinity between the initiation codon and the anticodon of the initiator Met-tRNAiMet. In some embodiments, the one or more chemically modified nucleotides is 2-thiouridine. In some embodiments, the one or more chemically modified nucleotides is 2′-O-methyl-2-thiouridine. In some embodiments, the one or more chemically modified nucleotides is 2-selenouridine. In some embodiments, the one or more chemically modified nucleotides is 2′-O-methyl ribose. In some embodiments, the one or more chemically modified nucleotides is selected from a locked nucleic acid, inosine, 2-methylguanosine, or 6-methyl-adenosine. In some embodiments, deoxyribonucleotides are incorporated into the modified mRNA.

A modified mRNA of the disclosure may include any suitable number of base pairs, including tens (e.g., 10, 20, 30, 40, 50, 60, 70, 80, 90 or 100), hundreds (e.g., 200, 300, 400, 500, 600, 700, 800, or 900) or thousands (e.g., 1000, 2000, 3000, 4000, 5000, 6000, 7000, 8000, 9000, 10,000) of base pairs. Any number (e.g., all, some, or none) of nucleobases, nucleosides, or nucleotides may be an analog of a canonical species, substituted, modified, or otherwise non-naturally occurring. In certain embodiments, all of a particular nucleobase type may be modified.

In some embodiments, a mRNA may instead or additionally include a chain terminating nucleoside. For example, a chain terminating nucleoside may include those nucleosides deoxygenated at the 2′ and/or 3′ positions of their sugar group. Such species may include 3′-deoxyadenosine (cordycepin), 3′-deoxyuridine, 3′-deoxycytosine, 3′-deoxyguanosine, 3′-deoxythymine, and 2′,3′-dideoxynucleosides, such as 2′,3′-dideoxyadenosine, 2′,3′-dideoxyuridine, 2′,3′-dideoxycytosine, 2′,3′-dideoxyguanosine, and 2′,3′-dideoxythymine. In some embodiments, incorporation of a chain terminating nucleotide into an mRNA, for example at the 3′-terminus, may result in stabilization of the mRNA, as described, for example, in International Patent Publication No. WO 2013/103659.

An mRNA may instead or additionally include a stem loop, such as a histone stem loop. A stem loop may include 2, 3, 4, 5, 6, 7, 8, or more nucleotide base pairs. For example, a stem loop may include 4, 5, 6, 7, or 8 nucleotide base pairs. A stem loop may be located in any region of an mRNA. For example, a stem loop may be located in, before, or after an untranslated region (a 5′ untranslated region or a 3′ untranslated region), a coding region, or a polyA sequence or tail. In some embodiments, a stem loop may affect one or more function(s) of an mRNA, such as initiation of translation, translation efficiency, and/or transcriptional termination.

In some embodiments, an mRNA includes one or more (e.g., 1, 2, 3 or 4) different modified nucleobases, nucleosides, or nucleotides. In some embodiments, an mRNA includes one or more (e.g., 1, 2, 3, 4, 5, 6, 7, 8, 9, 10, 20, 30, 40, 50, 60, 70, 80, 90, 100, or more) different modified nucleobases, nucleosides, or nucleotides. In some embodiments, the modified mRNA may have reduced degradation in a cell into which the mRNA is introduced, relative to a corresponding unmodified mRNA.

In some embodiments, the modified nucleobase is a modified uracil. Exemplary nucleobases and nucleosides having a modified uracil include pseudouridine (y), pyridin-4-one ribonucleoside, 5-aza-uridine, 6-aza-uridine, 2-thio-5-aza-uridine, 2-thio-uridine (s²U), 4-thio-uridine (s⁴U), 4-thio-pseudouridine, 2-thio-pseudouridine, 5-hydroxy-uridine (hoSU), 5-aminoallyl-uridine, 5-halo-uridine (e.g., 5-iodo-uridineor 5-bromo-uridine), 3-methyl-uridine (m³U), 5-methoxy-uridine (mo⁵U), uridine 5-oxyacetic acid (cmo⁵U), uridine 5-oxyacetic acid methyl ester (momo⁵U), 5-carboxymethyl-uridine (cm⁵U), 1-carboxymethyl-pseudouridine, 5- carboxyhydroxymethyl-uridine (chm⁵U). 5-carboxyhydroxymethyl-uridine methyl ester (mchm⁵U), 5-methoxycarbonylmethyl-uridine (mcmSU), 5-methoxycarbonylmethyl-2-thio-uridine (mcmas2U), 5-aminomethyl-2-thio-uridine (mnm⁵s^{2U}), 5-methylaminomethyl-uridine (mnm⁵U), 5-methylaminomethyl-2-thio-uridine (mnm⁵s²U), 5-methylaminomethyl-2-selenouridine (mnm⁵s²U), 5-carbamoylmethyl-uridine (ncm⁵U), 5-carboxymethylaminomethyl-uridine (cmnm⁵U), 5-carboxymethylaminomethyl-2-thio-uridine (cmnm⁵s²U), 5-propynyl-uridine, 1-propynyl-pseudouridine, 5-taurinomethyl-uridine (τm^{S}U), 1-taurinomethyl-pseudouridine, 5-taurinomethyl-2-thio-uridine(τm⁵s2U), 1-taurinomethyl-4-thio-pseudouridine, 5-methyl-uridine (m⁵U, i.e., having the nucleobase deoxythymine), 1-methyl-pseudouridine (m¹ψ), 5-methyl-2-thio-uridine (m5s²U), 1-methyl-4-thio-pseudouridine (m¹s⁴ψ), 4-thio-1-methyl-pseudouridine, 3-methyl-pseudouridine (m³ψ), 2-thio-1-methyl-pseudouridine, 1-methyl-1-deaza-pseudouridine, 2-thio-1-methyl-1-deaza-pseudouridine, dihydrouridine (D), dihydropseudouridine, 5,6-dihydrouridine, 5-methyl-dihydrouridine (m5D), 2-thio-dihydrouridine, 2-thio-dihydropseudouridine, 2-methoxy-uridine, 2-methoxy-4-thio-uridine, 4- methoxy-pseudouridine, 4-methoxy-2-thio-pseudouridine, N1-methyl-pseudouridine, 3-(3-amino-3-carboxypropyl)uridine (acp³U), 1-methyl-3-(3-amino-3-carboxypropyl)pseudouridine (acp³ψ), 5-(isopentenylaminomethyl)uridine (inm⁵S), 5-(isopentenylaminomethyl)-2-thio-uridine (inm⁵s2U), α-thio-uridine, 2′-O-methyl-uridine (Um), 5,2′-O-dimethyl-uridine (m⁵Um), 2′-O-methyl-pseudouridine (ψm), 2-thio-2′-O-methyl-uridine (s²Um), 5-methoxycarbonylmethyl-2′-O-methyl-uridine (mcm⁵Um), 5-carbamoylmethyl-2′-O-methyl-uridine (ncm⁵Um), 5-carboxymethylaminomethyl-2′-O-methyl-uridine (cmnm⁵Um), 3,2′-O-dimethyl-uridine (m³Um), and 5-(isopentenylaminomethyl)-2′-O-methyl-uridine (inm⁵Um), 1-thio-uridine, deoxythymidine, 2′-F-ara-uridine, 2′-F-uridine, 2′-OH-ara-uridine, 5-(2-carbomethoxyvinyl) uridine, and 5-[3-(1-E-propenylamino)] uridine.

In some embodiments, the modified nucleobase is a modified cytosine. Exemplary nucleobases and nucleosides having a modified cytosine include 5-aza-cytidine, 6-aza-cytidine, pseudoisocytidine, 3-methyl-cytidine (m³C), N4-acetyl-cytidine (ac⁴C), 5-formyl-cytidine (f⁵C), N4-methyl-cytidine (m⁴C), 5-methyl-cytidine (m⁵C), 5-halo- cytidine (e.g., 5-iodo-cytidine), 5-hydroxymethyl-cytidine (hm⁵C). 1-methyl-pseudoisocytidine, pyrrolo-cytidine, pyrrolo-pseudoisocytidine, 2-thio-cytidine (s²C), 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, lysidine (k₂C), α-thio-cytidine, 2′-O-methyl-cytidine (Cm), 5,2′-O-dimethyl-cytidine (m^{S}Cm), N4-acetyl-2′-O-methyl-cytidine (ac⁴Cm), N4,2′-O-dimethyl-cytidine (m⁴Cm), 5-formyl-2′-O-methyl-cytidine (f⁵Cm), N4,N4,2′-O-trimethyl-cytidine (m⁴²Cm), 1-thio-cytidine, 2′-F-ara-cytidine, 2′-F-cytidine, and 2′-OH-ara-cytidine.

In some embodiments, the modified nucleobase is a modified adenine. Exemplary nucleobases and nucleosides having a modified adenine include α-thio-adenosine, 2-amino-purine, 2, 6-diaminopurine, 2-amino-6-halo-purine (e.g., 2-amino-6-chloro-purine), 6-halo-purine (e.g., 6-chloro-purine), 2-amino-6-methyl-purine, 8-azido-adenosine, 7-deaza-adenine, 7-deaza-8-aza-adenine, 7-deaza-2-amino-purine, 7-deaza-8-aza-2-amino- purine, 7-deaza-2,6-diaminopurine, 7-deaza-8-aza-2,6-diaminopurine, 1-methyl-adenosine (m¹A), 2-methyl-adenine (m²A), N6-methyl-adenosine (m⁶A), 2-methylthio-N6-methyl-adenosine (ms.sup.²m⁶A), N6-isopentenyl-adenosine (i⁶A), 2-methylthio-N6-isopentenyl-adenosine (ms²i6A), N6-(cis-hydroxyisopentenyl)adenosine (io⁶A), 2-methylthio-N6-(cis-hydroxyisopentenyl)adenosine (ms²io⁶A), N6-glycinylcarbamoyl-adenosine (g5A), N6-threonylcarbamoyl-adenosine (t⁶A), N6-methyl-N6-threonylcarbamoyl-adenosine (m⁶t⁶A), 2-methylthio-N6-threonylcarbamoyl-adenosine (ms²g⁶A), N6, N6-dimethyl-adenosine (m⁶²A), N6-hydroxynorvalylcarbamoyl-adenosine (hn⁶A), 2-methylthio-N6-hydroxynorvalylcarbamoyl-adenosine (ms²hn⁵A), N6-acetyl-adenosine (ac⁶A), 7-methyl-adenine, 2-methylthio-adenine, 2-methoxy-adenine, α-thio-adenosine, 2′-O-methyl-adenosine (Am), N6,2′-O-dimethyl-adenosine (m⁶Am), N6, N6,2′-O-trimethyl-adenosine (m⁶²Am), 1,2′-O-dimethyl-adenosine (m¹Am). 2′-O-ribosyladenosine (phosphate) (Ar(p)), 2-amino-N6-methyl-purine, 1-thio-adenosine, 8-azido-adenosine, 2′-F-ara-adenosine, 2′-F-adenosine, 2′-OH-ara- adenosine, and N6-(19-amino-pentaoxanonadecyl)-adenosine.

In some embodiments, the modified nucleobase is a modified guanine. Exemplary nucleobases and nucleosides having a modified guanine include α-thio-guanosine, inosine (1), 1-methyl-inosine (m¹I), wyosine (imG), methylwyosine (mimG), 4-demethyl-wyosine (imG-14), isowyosine (imG2), wybutosine (yW), peroxywybutosine (o2yW), hydroxywybutosine (OhyW), undermodified hydroxywybutosine (OhyW*), 7-deaza-guanosine, queuosine (Q), epoxyqueuosine (oQ), galactosyl-queuosine (galQ), mannosyl-queuosine (manQ), 7-cyano-7-deaza-guanosine (preQo), 7-aminomethyl-7-deaza-guanosine (preQi), archaeosine (G+), 7-deaza-8-aza-guanosine, 6-thio-guanosine, 6-thio-7-deaza-guanosine, 6-thio-7-deaza-8-aza- guanosine, 7-methyl-guanosine (m.sup.7G), 6-thio-7-methyl-guanosine, 7-methyl-inosine, 6-methoxy-guanosine, 1-methyl-guanosine (mG), N2-methyl-guanosine (m²G), N2,N2-dimethyl-guanosine (m²²G), N2,7-dimethyl-guanosine (m²′7G), N2, N2,7-dimethyl-guanosine (m²′2′⁷G), 8-oxo-guanosine, 7-methyl-8-oxo-guanosine, 1-methyl-6-thio-guanosine, N2-methyl-6-thio-guanosine, N2,N2-dimethyl-6-thio-guanosine, α-thio-guanosine, 2′-O-methyl-guanosine (Gm), N2-methyl-2′-O-methyl-guanosine (m²Gm), N2,N2-dimethyl-2′-O-methyl-guanosine (m²²Gm), 1-methyl-2′-O-methyl-guanosine (mGm), N2,7-dimethyl-2′-O-methyl-guanosine (m^{2,7}Gm), 2′-O-methyl-inosine (Im), 1,2′-O-dimethyl-inosine (m¹im), 2′-O-ribosylguanosine (phosphate) (Gr(p)). 1-thio-guanosine, 06-methyl-guanosine, 2′-F-ara-guanosine, and 2′-F-guanosine.

In some embodiments, an mRNA of the disclosure includes a combination of one or more of the aforementioned modified nucleobases (e.g., a combination of 2, 3 or 4 of the aforementioned modified nucleobases.)

In some embodiments, the modified nucleobase is pseudouridine (ψ), N1-methylpseudouridine (m¹ψ), 2-thiouridine, 4′-thiouridine, 5-methylcytosine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine, or 2′-O-methyl uridine. In some embodiments, an mRNA of the disclosure includes a combination of one or more of the aforementioned modified nucleobases (e.g., a combination of 2, 3 or 4 of the aforementioned modified nucleobases.)

In some embodiments, the modified nucleobase is a modified cytosine. Exemplary nucleobases and nucleosides having a modified cytosine include N4-acetyl-cytidine (ac⁴C), 5-methyl-cytidine (m⁵C), 5-halo-cytidine (e.g., 5-iodo-cytidine), 5-hydroxymethyl-cytidine (hm⁵C), 1-methyl-pseudoisocytidine, 2-thio-cytidine (s²C), 2-thio-5-methyl-cytidine. In some embodiments, an mRNA of the disclosure includes a combination of one or more of the aforementioned modified nucleobases (e.g., a combination of 2, 3 or 4 of the aforementioned modified nucleobases.)

In some embodiments, the modified nucleobase is a modified adenine. Exemplary nucleobases and nucleosides having a modified adenine include 7-deaza-adenine, 1-methyl-adenosine (m¹A), 2-methyl-adenine (m²A), N6-methyl-adenosine (m⁵A). In some embodiments, an mRNA of the disclosure includes a combination of one or more of the aforementioned modified nucleobases (e.g., a combination of 2, 3 or 4 of the aforementioned modified nucleobases.) In some embodiments, the modified nucleobase is a modified guanine. Exemplary nucleobases and nucleosides having a modified guanine include inosine (I), 1-methyl-inosine (m¹I), wyosine (imG), methylwyosine (mimG), 7-deaza-guanosine, 7-cyano-7-deaza-guanosine (preQo), 7-aminomethyl-7-deaza-guanosine (preQi), 7-methyl-guanosine (m⁷G), 1-methyl-guanosine (mG), 8-oxo-guanosine, 7-methyl-8-oxo-guanosine. In some embodiments, an mRNA of the disclosure includes a combination of one or more of the aforementioned modified nucleobases (e.g., a combination of 2, 3 or 4 of the aforementioned modified nucleobases.)

In some embodiments, the modified nucleobase is 1-methyl-pseudouridine (mψ), 5-methoxy-uridine (mo⁵U), 5-methyl-cytidine (m⁵C), pseudouridine (ψ), α-thio-guanosine, or α-thio- adenosine. In some embodiments, an mRNA of the disclosure includes a combination of one or more of the aforementioned modified nucleobases (e.g., a combination of 2, 3 or 4 of the aforementioned modified nucleobases.)

In some embodiments, the mRNA comprises pseudouridine (ψ). In some embodiments, the mRNA comprises pseudouridine (ψ) and 5-methyl-cytidine (m⁵C). In some embodiments, the mRNA comprises 1-methyl-pseudouridine (m¹ψ). In some embodiments, the mRNA comprises 1-methyl-pseudouridine (m¹ψ) and 5-methyl-cytidine (m⁵C). In some embodiments, the mRNA comprises 2-thiouridine (s²U). In some embodiments, the mRNA comprises 2-thiouridine and 5-methyl-cytidine (m⁵C). In some embodiments, the mRNA comprises 5-methoxy-uridine (mo⁵U). In some embodiments, the mRNA comprises 5-methoxy-uridine (moSU) and 5-methyl-cytidine (m⁵C). In some embodiments, the mRNA comprises 2′-O-methyl uridine. In some embodiments, the mRNA comprises 2′-O-methyl uridine and 5-methyl-cytidine (m⁵C). In some embodiments, the mRNA comprises N6-methyl-adenosine (m⁶A). In some embodiments, the mRNA comprises N6-methyl-adenosine (m.sup.6A) and 5-methyl-cytidine (m⁵C).

In certain embodiments, an mRNA of the disclosure is uniformly modified (i.e., fully modified, modified through-out the entire sequence) for a particular modification. For example, an mRNA can be uniformly modified with 5-methyl-cytidine (m⁵C), meaning that all cytosine residues in the mRNA sequence are replaced with 5-methyl-cytidine (m⁵C). Similarly, mRNAs of the disclosure can be uniformly modified for any type of nucleoside residue present in the sequence by replacement with a modified residue such as those set forth above.

In some embodiments, an mRNA of the disclosure may be modified in a coding region (e.g., an open reading frame encoding a polypeptide). In other embodiments, an mRNA may be modified in regions besides a coding region. For example, in some embodiments, a 5′-UTR and/or a 3′-UTR are provided, wherein either or both may independently contain one or more different nucleoside modifications. In such embodiments, nucleoside modifications may also be present in the coding region,

### MicroRNA (miRNA) Binding Sites

Polynucleotides of the disclosure include microRNA (miRNA) binding sites In some embodiments, a polynucleotide (e.g., a ribonucleic acid (RNA), e.g., a messenger RNA (mRNA)) of the disclosure comprises an open reading frame (ORF) encoding a polypeptide of interest and further comprises one or more miRNA binding site(s) in he 5′UTR. Inclusion or incorporation of miRNA binding site(s) provides for regulation of polynucleotides of the disclosure, and in turn, of the polypeptides encoded therefrom, based on tissue-specific and/or cell-type specific expression of naturally-occurring miRNAs.

A miRNA, e.g., a natural-occurring miRNA, is a 19-25 nucleotide long noncoding RNA that binds to a polynucleotide and down-regulates gene expression either by reducing stability or by inhibiting translation of the polynucleotide. A miRNA sequence comprises a “seed” region, i.e., a sequence in the region of positions 2-8 of the mature miRNA. A miRNA seed can comprise positions 2-8 or 2-7 of the mature miRNA. In some embodiments, a miRNA seed can comprise 7 nucleotides (e.g., nucleotides 2-8 of the mature miRNA), wherein the seed-complementary site in the corresponding miRNA binding site is flanked by an adenosine (A) opposed to miRNA position 1. In some embodiments, a miRNA seed can comprise 6 nucleotides (e.g., nucleotides 2-7 of the mature miRNA), wherein the seed-complementary site in the corresponding miRNA binding site is flanked by an adenosine (A) opposed to miRNA position 1. See, for example, Grimson A, Farh K K, Johnston W K, Garrett-Engele P, Lim L P, Bartel D P; Mol Cell. 2007 Jul. 6; 27(1):91-105. miRNA profiling of the target cells or tissues can be conducted to determine the presence or absence of miRNA in the cells or tissues. In some embodiments, a polynucleotide (e.g., a ribonucleic acid (RNA), e.g., a messenger RNA (mRNA)) of the disclosure comprises one or more microRNA binding sites (microRNA target sequences which are microRNA complementary sequences including microRNA seed complementary sequences).

As used herein, the term “microRNA (miRNA or miR) binding site” refers to a sequence within a polynucleotide, e.g., within a DNA or within an RNA transcript, including in the 5′UTR, e.g., mRNA may comprise one or more miRNA binding site(s).

The miRNA binding site can have complementarity to, for example, a 19-25 nucleotide miRNA sequence, to a 19-23 nucleotide miRNA sequence, or to a 22 nucleotide miRNA sequence. A miRNA binding site can be complementary to only a portion of a miRNA, e.g., to a portion less than 1, 2, 3, or 4 nucleotides of the full length of a naturally-occurring miRNA sequence, e.g., to 2 to 10 nucleotides of a seed region.

In some embodiments, a miRNA binding site includes a sequence that has complementarity (e.g., partial or complete complementarity) with a miRNA seed sequence. In some embodiments, the miRNA binding site includes a sequence that has complete complementarity with a miRNA seed sequence. In some embodiments, a miRNA binding site includes a sequence that has complementarity (e.g., partial or complete complementarity) with an miRNA sequence. In some embodiments, the miRNA binding site includes a sequence that has complete complementarity with a miRNA sequence. In some embodiments, a miRNA binding site has complete complementarity with a miRNA sequence but for 1, 2, or 3 nucleotide substitutions, terminal additions, and/or truncations.

In some embodiments, the miRNA binding site is the same length as the corresponding miRNA. In other embodiments, the miRNA binding site is one, two, three, four, five, six, seven, eight, nine, ten, eleven or twelve nucleotide(s) shorter than the corresponding miRNA at the 5′ terminus, the 3′ terminus, or both. In still other embodiments, the microRNA binding site is two nucleotides shorter than the corresponding microRNA at the 5′ terminus, the 3′ terminus, or both. The miRNA binding sites that are shorter than the corresponding miRNAs are still capable of degrading the mRNA incorporating one or more of the miRNA binding sites or preventing the mRNA from translation,

In some embodiments, the miRNA binding site has one, two, three, four, five, six, seven, eight, nine, ten, eleven or twelve mismatch(es) from the corresponding miRNA, e.g., one, two, three, four, or five mismatches relative to the seed sequence of the miRNA.

In some embodiments, the miRNA binding site has at least about ten, at least about eleven, at least about twelve, at least about thirteen, at least about fourteen, at least about fifteen, at least about sixteen, at least about seventeen, at least about eighteen, at least about nineteen, at least about twenty, or at least about twenty-one contiguous nucleotides complementary to at least about ten, at least about eleven, at least about twelve, at least about thirteen, at least about fourteen, at least about fifteen, at least about sixteen, at least about seventeen, at least about eighteen, at least about nineteen, at least about twenty, or at least about twenty-one, respectively, contiguous nucleotides of the corresponding miRNA.

In one embodiment, a polynucleotide of the disclosure can include two, three, four, five, six, seven, eight, nine, ten, or more miRNA-binding sites in the 5′-UTR.

In one embodiment, a polynucleotide of the disclosure has less than five, four, three or two miRNA-binding sites in the 5′-UTR.

At least one miRNA binding site can be engineered into the 5′UTR of a polynucleotide of the disclosure. In this context, at least two, at least three, at least four, at least five, at least six, at least seven, at least eight, at least nine, at least ten, or more miRNA binding sites can be engineered into a 5′UTR of a polynucleotide of the disclosure. In one embodiment, miRNA binding sites incorporated into a polynucleotide of the disclosure can be the same or can be different miRNA sites. A combination of different miRNA binding sites incorporated into a polynucleotide of the disclosure can include combinations in which more than one copy of any of the different miRNA sites are incorporated. In another embodiment, miRNA binding sites incorporated into a polynucleotide of the disclosure can target the same or different tissues in the body.

In some embodiments, a polynucleotide of the disclosure can be designed to incorporate miRNA binding sites that either have 100% identity to the complement of a known miRNA seed sequences or have less than 100% identity t the complement of a miRNA seed sequences. In some embodiments, a polynucleotide of the disclosure can be designed to incorporate miRNA binding sites that have at least: 60%, 65%, 70%, 75%, 80%, 85%, 90%, 95%, 96%, 97%, 98%, or 99% identity to the complement of a known miRNA seed sequences. The miRNA seed sequence can be partially mutated. In essence, the degree of match or mis-match between the miRNA binding site and the miRNA seed may act as a rheostat to more finely tune the ability of the miRNA to modulate protein expression. In addition, mutation in the non-seed region of a miRNA binding site can also impact the ability of a miRNA to modulate protein expression.

In one embodiment, a miRNA binding site sequence can be incorporated into the loop of a stem loop, e.g.,incorporated into the 5′ or 3′ stem of the stem loop.

In one embodiment, the 5′-UTR of a polynucleotide of the disclosure can comprise at least one miRNA binding site sequence which optionally includes the complement of a seed sequence, e.g., a miRNA binding site sequence that has at least 7, 8, 9, 10, 11 or 12 nucleotides having 0, 1, 2, 3, 4, or 5 mismatches to the complement of a corresponding miRNA. The miRNA sequence can be, but is not limited to, a 19 or 22 nucleotide sequence and/or a miRNA sequence without the seed.

### Delivery Agents

a. Lipid Compound

The present disclosure provides pharmaceutical compositions with advantageous properties. The lipid compositions described herein may be advantageously used in lipid nanoparticle compositions for the delivery of therapeutic and/or prophylactic agents, e.g., mRNAs, to mammalian cells or organs. For example, the lipids described herein have little or no immunogenicity. For example, the lipid compounds disclosed herein have a lower immunogenicity as compared to a reference lipid (e.g., MC3, KC2, or DLinDMA). For example, a formulation comprising a lipid disclosed herein and a therapeutic or prophylactic agent, e.g., mRNA, has an increased therapeutic index as compared to a corresponding formulation which comprises a reference lipid (e.g., MC3, KC2, or DLinDMA) and the same therapeutic or prophylactic agent.

In certain embodiments, the present application provides pharmaceutical compositions comprising:
(a) a polynucleotide comprising a nucleotide sequence encoding a polypeptide; and
(b) a delivery agent.

### Lipid Nanoparticle Formulations

In some embodiments, nucleic acids of the disclosure (e.g., mRNA) are formulated in a lipid nanoparticle (LNP). Lipid nanoparticles typically comprise ionizable cationic lipid, non-cationic lipid, sterol and PEG lipid components along with the nucleic acid cargo of interest. The lipid nanoparticles of the disclosure can be generated using components, compositions, and methods as are generally known in the art, see for example PCT/US2016/052352; PCT/US2016/068300; PCT/US2017/037551; PCT/US2015/027400; PCT/US2016/047406; PCT/US2016000129; PCT/US2016/014280; PCT/US2016/014280; PCT/US2017/038426; PCT/US2014/027077; PCT/US2014/055394; PCT/US2016/52117; PCT/US2012/069610; PCT/US2017/027492; PCT/US2016/059575 and PCT/US2016/069491 all of which are incorporated by reference herein in their entirety.

Nucleic acids of the present disclosure (e.g., mRNA) are typically formulated in lipid nanoparticle. In some embodiments, the lipid nanoparticle comprises at least one ionizable cationic lipid, at least one non-cationic lipid, at least one sterol, and/or at least one polyethylene glycol (PEG)-modified lipid.

In some embodiments, the lipid nanoparticle comprises a molar ratio of 20-60% ionizable cationic lipid. For example, the lipid nanoparticle may comprise a molar ratio of 20-50%, 20-40%, 20-30%, 30-60%, 30-50%, 30-40%, 40-60%, 40-50%, or 50-60% ionizable cationic lipid. In some embodiments, the lipid nanoparticle comprises a molar ratio of 20%, 30%, 40%, 50, or 60% ionizable cationic lipid.

In some embodiments, the lipid nanoparticle comprises a molar ratio of 5-25% non-cationic lipid. For example, the lipid nanoparticle may comprise a molar ratio of 5-20%, 5-15%, 5-10%, 10-25%, 10-20%, 10-25%, 15-25%, 15-20%, or 20-25% non-cationic lipid. In some embodiments, the lipid nanoparticle comprises a molar ratio of 5%, 10%, 15%, 20%, or 25% non-cationic lipid.

In some embodiments, the lipid nanoparticle comprises a molar ratio of 25-55% sterol. For example, the lipid nanoparticle may comprise a molar ratio of 25-50%, 25-45%, 25-40%, 25-35%, 25-30%, 30-55%, 30-50%, 30-45%, 30-40%, 30-35%, 35-55%, 35-50%, 35-45%, 35-40%, 40-55%, 40-50%, 40-45%, 45-55%, 45-50%, or 50-55% sterol, In some embodiments, the lipid nanoparticle comprises a molar ratio of 25%, 30%, 35%, 40%, 45%, 50%, or 55% sterol.

In some embodiments, the lipid nanoparticle comprises a molar ratio of 0.5-15% PEG-modified lipid. For example, the lipid nanoparticle may comprise a molar ratio of 0.5-10%, 0.5-5%, 1-15%, 1-10%, 1-5%, 2-15%, 2-10%, 2-5%, 5-15%, 5-10%, or 10-15%. In some embodiments, the lipid nanoparticle comprises a molar ratio of 0.5%, 1%, 2%, 3%, 4%, 5%, 6%, 7%, 8%, 9%, 10%, 11%, 12%, 13%, 14%, or 15% PEG-modified lipid.

In some embodiments, the lipid nanoparticle comprises a molar ratio of 20-60% ionizable cationic lipid, 5-25% non-cationic lipid, 25-55% sterol, and 0.5-15% PEG-modified lipid,

### Phospholipids

The lipid composition of the lipid nanoparticle composition disclosed herein can comprise one or more phospholipids, for example, one or more saturated or (poly)unsaturated phospholipids or a combination thereof. In general, phospholipids comprise a phospholipid moiety and one or more fatty acid moieties.

A phospholipid moiety can be selected, for example, from the non-limiting group consisting of phosphatidyl choline, phosphatidyl ethanolamine, phosphatidyl glycerol, phosphatidyl serine, phosphatidic acid, 2-lysophosphatidyl choline, and a sphingomyelin.

A fatty acid moiety can be selected, for example, from the non-limiting group consisting of lauric acid, myristic acid, myristoleic acid, palmitic acid, palmitoleic acid, stearic acid, oleic acid, linoleic acid, alpha-linolenic acid, erucic acid, phytanoic acid, arachidic acid, arachidonic acid, eicosapentaenoic acid, behenic acid, docosapentaenoic acid, and docosahexaenoic acid.

Particular phospholipids can facilitate fusion to a membrane. For example, a cationic phospholipid can interact with one or more negatively charged phospholipids of a membrane (e.g., a cellular or intracellular membrane). Fusion of a phospholipid to a membrane can allow one or more elements (e.g., a therapeutic agent) of a lipid-containing composition (e.g., LNPs) to pass through the membrane permitting, e.g., delivery of the one or more elements to a target tissue.

Non-natural phospholipid species including natural species with modifications and substitutions including branching, oxidation, cyclization, and alkynes are also contemplated. For example, a phospholipid can be functionalized with or cross-linked to one or more alkynes (e.g., an alkenyl group in which one or more double bonds is replaced with a triple bond). Under appropriate reaction conditions, an alkyne group can undergo a copper-catalyzed cycloaddition upon exposure to an azide. Such reactions can be useful in functionalizing a lipid bilayer of a nanoparticle composition to facilitate membrane permeation or cellular recognition or in conjugating a nanoparticle composition to a useful component such as a targeting or imaging moiety (e.g., a dye).

Phospholipids include, but are not limited to, glycerophospholipids such as phosphatidylcholines, phosphatidylethanolamines, phosphatidylserines, phosphatidylinositols, phosphatidy glycerols, and phosphatidic acids. Phospholipids also include phosphosphingolipid, such as sphingomyelin.

In some embodiments, a phospholipid of the disclosure comprises 1,2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-gly cero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2 cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, and mixtures thereof.

### Alternative Lipids

In certain embodiments, a phospholipid useful or potentially useful in the present disclosure comprises a modified phosphocholine moiety, wherein the alkyl chain linking the quaternary amine to the phosphoryl group is not ethylene (e.g., n is not 2). Therefore, in certain embodiments, a phospholipid useful.

In certain embodiments, an alternative lipid is used in place of a phospholipid of the present disclosure.

In certain embodiments, an alternative lipid of the disclosure is oleic acid.

### Structural Lipids

The lipid composition of a pharmaceutical composition disclosed herein can comprise one or more structural lipids. As used herein, the term “structural lipid” refers to sterols and also to lipids containing sterol moieties.

Incorporation of structural lipids in the lipid nanoparticle may help mitigate aggregation of other lipids in the particle. Structural lipids can be selected from the group including but not limited to, cholesterol, fecosterol, sitosterol, ergosterol, campesterol, stigmasterol, brassicasterol, tomatidine, tomatine, ursolic acid, alpha-tocopherol, hopanoids, phytosterols, steroids, and mixtures thereof. In some embodiments, the structural lipid is a sterol. As defined herein, “sterols” are a subgroup of steroids consisting of steroid alcohols. In certain embodiments, the structural lipid is a steroid. In certain embodiments, the structural lipid is cholesterol. In certain embodiments, the structural lipid is an analog of cholesterol. In certain embodiments, the structural lipid is alpha-tocopherol.

In some embodiments, the structural lipids may be one or more of the structural lipids described in U.S. Application No. 62/520,530.

### Polyethylene Glycol (PEG)-Lipids

The lipid composition of a pharmaceutical composition disclosed herein can comprise one or more a polyethylene glycol (PEG) lipid.

As used herein, the term “PEG-lipid” refers to polyethylene glycol (PEG)-modified lipids. Non-limiting examples of PEG-lipids include PEG-modified phosphatidylethanolamine and phosphatidic acid, PEG-ceramide conjugates (e.g., PEG-CerC14 or PEG-CerC20), PEG-modified dialkylamines and PEG-modified 1,2-diacyloxypropan-3-amines. Such lipids are also referred to as PEGylated lipids, For example, a PEG lipid can be PEG-c-DOMG, PEG-DMG, PEG-DLPE, PEG-DMPE, PEG-DPPC, or a PEG-DSPE lipid.

In some embodiments, the PEG-lipid includes, but not limited to 1,2-dimyristoyl-sn-glycerol methoxypolyethylene glycol (PEG-DMG), 1,2-distearoyl-sn-glycero-3-phosphoethanolamine-N-[amino(polyethylene glycol)] (PEG-DSPE), PEG-disteryl glycerol (PEG-DSG), PEG-dipalmetoleyl, PEG-dioleyl, PEG-distearyl, PEG-diacylglycamide (PEG-DAG), PEG-dipalmitoyl phosphatidylethanolamine (PEG-DPPE), or PEG-1,2-dimyristyloxipropyl-3-amine (PEG-c-DMA).

In one embodiment, the PEG-lipid is selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and mixtures thereof.

In some embodiments, the lipid moiety of the PEG-lipids includes those having lengths of from about C14 to about C22, or about C14 to about C16. In some embodiments, a PEG moiety, for example an mPEG-NH.sub.2, has a size of about 1000, 2000, 5000, 10,000, 15,000 or 20,000 daltons. In one embodiment, the PEG-lipid is PEG2k-DMG.

In one embodiment, the lipid nanoparticles described herein can comprise a PEG lipid which is a non-diffusible PEG. Non-limiting examples of non-diffusible PEGs include PEG-DSG and PEG-DSPE.

PEG-lipids are known in the art, such as those described in U.S. Pat. No. 8,158,601 and International Publ. No. WO 2015/130584 A2, which are incorporated herein by reference in their entirety.

In general, some of the other lipid components (e.g., PEG lipids) of various formulae, described herein may be synthesized as described International Patent Application No. PCT/US2016/000129, filed Dec. 10, 2016, entitled “Compositions and Methods for Delivery of Therapeutic Agents,” which is incorporated by reference in its entirety.

The lipid component of a lipid nanoparticle composition may include one or more molecules comprising polyethylene glycol, such as PEG or PEG-modified lipids. Such species may be alternately referred to as PEGylated lipids. A PEG lipid is a lipid modified with polyethylene glycol. A PEG lipid may be selected from the non-limiting group including PEG-modified phosphatidylethanolamines, PEG-modified phosphatidic acids, PEG-modified ceramides, PEG-modified dialkylamines, PEG-modified diacylglycerols, PEG-modified dialkylglycerols, and mixtures thereof. For example, a PEG lipid may be PEG-c-DOMG, PEG-DMG, PEG-DLPE, PEG-DMPE, PEG-DPPC, or a PEG-DSPE lipid.

In some embodiments, a LNP of the disclosure comprises an ionizable cationic lipid of a phospholipid comprising DOPE, a structural lipid comprising cholesterol, and a PEG lipid comprising a compound having Formula VII.

In some embodiments, a LNP of the disclosure comprises an N:P ratio of from about 2:1 to about 30:1.

In some embodiments, a LNP of the disclosure comprises an N:P ratio of about 6:1.

In some embodiments, a LNP of the disclosure comprises an N:P ratio of about 3:1.

In some embodiments, a LNP of the disclosure comprises a wt/wt ratio of the ionizable cationic lipid component to the RNA of from about 10:1 to about 100:1.

In some embodiments, a LNP of the disclosure comprises a wt/wt ratio of the ionizable cationic lipid component to the RNA of about 20:1.

In some embodiments, a LNP of the disclosure comprises a wt/wt ratio of the ionizable cationic lipid component to the RNA of about 10:1.

In some embodiments, a LNP of the disclosure has a mean diameter from about 50 nm to about 150 nm.

In some embodiments, a LNP of the disclosure has a mean diameter from about 70 nm to about 120 nm.

### Nanoparticle Compositions

In some embodiments, the pharmaceutical compositions disclosed herein are formulated as lipid nanoparticles (LNP). Accordingly, the present disclosure also provides nanoparticle compositions comprising (i) a lipid composition comprising a delivery agent such as compound as described herein, and (ii) a polynucleotide encoding a polypeptide, e.g., an isolated mRNA or vector as described herein. In such nanoparticle composition, the lipid composition disclosed herein can encapsulate the polynucleotide encoding a polypeptide.

Nanoparticle compositions are typically sized on the order of micrometers or smaller and can include a lipid bilayer. Nanoparticle compositions encompass lipid nanoparticles (LNPs), liposomes (e.g., lipid vesicles), and lipoplexes. For example, a nanoparticle composition can be a liposome having a lipid bilayer with a diameter of 500 nm or less.

Nanoparticle compositions include, for example, lipid nanoparticles (LNPs), liposomes, and lipoplexes. In some embodiments, nanoparticle compositions are vesicles including one or more lipid bilayers. In certain embodiments, a nanoparticle composition includes two or more concentric bilayers separated by aqueous compartments. Lipid bilayers can be functionalized and/or crosslinked to one another. Lipid bilayers can include one or more ligands, proteins, or channels.

In one embodiment, a lipid nanoparticle comprises an ionizable lipid, a structural lipid, a phospholipid, and mRNA. In some embodiments, the LNP comprises an ionizable lipid, a PEG-modified lipid, a sterol and a structural lipid. In some embodiments, the LNP has a molar ratio of about 20-60% ionizable lipid:about 5-25% structural lipid:about 25-55% sterol; and about 0.5-15% PEG-modified lipid.

In some embodiments, the LNP has a polydispersity value of less than 0.4. In some embodiments, the LNP has a net neutral charge at a neutral pH. In some embodiments, the LNP has a mean diameter of 50-150 nm. In some embodiments, the LNP has a mean diameter of 80-100 nm.

As generally defined herein, the term “lipid” refers to a small molecule that has hydrophobic or amphiphilic properties. Lipids may be naturally occurring or synthetic. Examples of classes of lipids include, but are not limited to, fats, waxes, sterol-containing metabolites, vitamins, fatty acids, glycerolipids, glycerophospholipids, sphingolipids, saccharolipids, and polyketides, and prenol lipids. In some instances, the amphiphilic properties of some lipids lead them to form liposomes, vesicles, or membranes in aqueous media.

In some embodiments, a lipid nanoparticle (LNP) may comprise an ionizable lipid. As used herein, the term “ionizable lipid” has its ordinary meaning in the art and may refer to a lipid comprising one or more charged moieties. In some embodiments, an ionizable lipid may be positively charged or negatively charged. An ionizable lipid may be positively charged, in which case it can be referred to as “cationic lipid”. In certain embodiments, an ionizable lipid molecule may comprise an amine group, and can be referred to as an ionizable amino lipid. As used herein, a “charged moiety” is a chemical moiety that carries a formal electronic charge, e.g., monovalent (+1, or −1), divalent (+2, or −2), trivalent (+3, or −3), etc. The charged moiety may be anionic (i.e., negatively charged) or cationic (i.e., positively charged). Examples of positively-charged moieties include amine groups (e.g., primary, secondary, and/or tertiary amines), ammonium groups, pyridinium group, guanidine groups, and imidizolium groups. In a particular embodiment, the charged moieties comprise amine groups. Examples of negatively-charged groups or precursors thereof, include carboxylate groups, sulfonate groups, sulfate groups, phosphonate groups, phosphate groups, hydroxyl groups, and the like. The charge of the charged moiety may vary, in some cases, with the environmental conditions, for example, changes in pH may alter the charge of the moiety, and/or cause the moiety to become charged or uncharged. In general, the charge density of the molecule may be selected as desired.

As used herein, “size” or “mean size” in the context of nanoparticle compositions refers to the mean diameter of a nanoparticle composition.

In one embodiment, the polynucleotide encoding a polypeptide is formulated in lipid nanoparticles having a diameter from about 10 to about 100 nm such as, but not limited to, about 10 to about 20 nm, about 10 to about 30 nm, about 10 to about 40 nm, about 10 to about 50 nm, about 10 to about 60 nm, about 10 to about 70 nm, about 10 to about 80 nm, about 10 to about 90 nm, about 20 to about 30 nm, about 20 to about 40 nm, about 20 to about 50 nm, about 20 to about 60 nm, about 20 to about 70 nm, about 20 to about 80 nm, about 20 to about 90 nm, about 20 to about 100 nm, about 30 to about 40 nm, about 30 to about 50 nm, about 30 to about 60 nm, about 30 to about 70 nm, about 30 to about 80 nm, about 30 to about 90 nm, about 30 to about 100 nm, about 40 to about 50 nm, about 40 to about 60 nm, about 40 to about 70 nm, about 40 to about 80 nm, about 40 to about 90 nm, about 40 to about 100 nm, about 50 to about 60 nm, about 50 to about 70 nm, about 50 to about 80 nm, about 50 to about 90 nm, about 50 to about 100 nm, about 60 to about 70 nm, about 60 to about 80 nm, about 60 to about 90 nm, about 60 to about 100 nm, about 70 to about 80 nm, about 70 to about 90 nm, about 70 to about 100 nm, about 80 to about 90 nm, about 80 to about 100 nm and/or about 90 to about 100 nm.

In one embodiment, the nanoparticles have a diameter from about 10 to 500 nm. In one embodiment, the nanoparticle has a diameter greater than 100 nm, greater than 150 nm, greater than 200 nm, greater than 250 nm, greater than 300 nm, greater than 350 nm, greater than 400 nm, greater than 450 nm, greater than 500 nm, greater than 550 nm, greater than 600 nm, greater than 650 nm, greater than 700 nm, greater than 750 nm, greater than 800 nm, greater than 850 nm, greater than 900 nm, greater than 950 nm or greater than 1000 nm.

In some embodiments, the largest dimension of a nanoparticle composition is 1 am or shorter (e.g., 1 am, 900 nm, 800 nm, 700 nm, 600 nm, 500 nm, 400 nm, 300 nm, 200 nm, 175 nm, 150 nm, 125 nm, 100 nm, 75 nm, 50 nm, or shorter).

A nanoparticle composition can be relatively homogenous. A polydispersity index can be used to indicate the homogeneity of a nanoparticle composition, e.g., the particle size distribution of the nanoparticle composition. A small (e.g., less than 0.3) polydispersity index generally indicates a narrow particle size distribution. A nanoparticle composition can have a polydispersity index from about 0 to about 0.25, such as 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, or 0.25. In some embodiments, the polydispersity index of a nanoparticle composition disclosed herein can be from about 0.10 to about 0.20.

The zeta potential of a nanoparticle composition can be used to indicate the electrokinetic potential of the composition. For example, the zeta potential can describe the surface charge of a nanoparticle composition. Nanoparticle compositions with relatively low charges, positive or negative, are generally desirable, as more highly charged species can interact undesirably with cells, tissues, and other elements in the body. In some embodiments, the zeta potential of a nanoparticle composition disclosed herein can be from about −10 mV to about +20 mV, from about −10 mV to about +15 mV, from about 10 mV to about +10 mV, from about −10 mV to about +5 mV, from about −10 mV to about 0 mV, from about −10 mV to about −5 mV, from about −5 mV to about +20 mV, from about −5 mV to about +15 mV, from about −5 mV to about +10 mV, from about −5 mV to about +5 mV, from about −5 mV to about 0 mV, from about 0 mV to about +20 mV, from about 0 mV to about +15 mV, from about 0 mV to about +10 mV, from about 0 mV to about +5 mV, from about +5 mV to about +20 mV, from about +5 mV to about +15 mV, or from about +5 mV to about +10 mV.

In some embodiments, the zeta potential of the lipid nanoparticles can be from about 0 mV to about 100 mV, from about 0 mV to about 90 mV, from about 0 mV to about 80 mV, from about 0 mV to about 70 mV, from about 0 mV to about 60 mV, from about 0 mV to about 50 mV, from about 0 mV to about 40 mV, from about 0 mV to about 30 mV, from about 0 mV to about 20 mV, from about 0 mV to about 10 mV, from about 10 mV to about 100 mV, from about 10 mV to about 90 mV, from about 10 mV to about 80 mV, from about 10 mV to about 70 mV, from about 10 mV to about 60 mV, from about 10 mV to about 50 mV, from about 10 mV to about 40 mV, from about 10 mV to about 30 mV, from about 10 mV to about 20 mV, from about 20 mV to about 100 mV, from about 20 mV to about 90 mV, from about 20 mV to about 80 mV, from about 20 mV to about 70 mV, from about 20 mV to about 60 mV, from about 20 mV to about 50 mV, from about 20 mV to about 40 mV, from about 20 mV to about 30 mV, from about 30 mV to about 100 mV, from about 30 mV to about 90 mV, from about 30 mV to about 80 mV, from about 30 mV to about 70 mV, from about 30 mV to about 60 mV, from about 30 mV to about 50 mV, from about 30 mV to about 40 mV, from about 40 mV to about 100 mV, from about 40 mV to about 90 mV, from about 40 mV to about 80 mV, from about 40 mV to about 70 mV, from about 40 mV to about 60 mV, and from about 40 mV to about 50 mV. In some embodiments, the zeta potential of the lipid nanoparticles can be from about 10 mV to about 50 mV, from about 15 mV to about 45 mV, from about 20 mV to about 40 mV, and from about 25 mV to about 35 mV. In some embodiments, the zeta potential of the lipid nanoparticles can be about 10 mV, about 20 mV, about 30 mV, about 40 mV, about 50 mV, about 60 mV, about 70 mV, about 80 mV, about 90 mV, and about 100 mV.

### Pharmaceutical Compositions

The present disclosure includes pharmaceutical compositions comprising an mRNA, a vector encoding the mRNA or a nanoparticle (e.g., a lipid nanoparticle) described herein, in combination with one or more pharmaceutically acceptable excipient, carrier or diluent. In particular embodiments, the mRNA is present in a nanoparticle, e.g., a lipid nanoparticle. In particular embodiments, the mRNA or nanoparticle is present in a pharmaceutical composition. In various embodiments, the one or more mRNA present in the pharmaceutical composition is encapsulated in a nanoparticle, e.g., a lipid nanoparticle. In particular embodiments, the molar ratio of the first mRNA to the second mRNA is about 1:50, about 1:25, about 1:10, about 1:5, about 1:4, about 1:3, about 1:2, about 1:1, about 2:1, about 3:1, about 4:1, or about 5:1, about 10:1, about 25:1 or about 50:1. In particular embodiments, the molar ratio of the first mRNA to the second mRNA is greater than 1:1.

In some embodiments, a composition described herein comprises an mRNA encoding a polypeptide or a vector encoding the mRNA. In some embodiments, the polypeptide is a therapeutic polypeptide. In some embodiments, the polypeptide is an enzyme. In some embodiments, the polypeptide is an antibody. In some embodiments, the polypeptide comprises an antigen.

Pharmaceutical compositions may optionally include one or more additional active substances, for example, therapeutically and/or prophylactically active substances. Pharmaceutical compositions of the present disclosure may be sterile and/or pyrogen-free. General considerations in the formulation and/or manufacture of pharmaceutical agents may be found, for example, in Remington: The Science and Practice of Pharmacy 21.sup.st ed., Lippincott Williams & Wilkins, 2005 (incorporated herein by reference in its entirety). In particular embodiments, a pharmaceutical composition comprises an mRNA and a lipid nanoparticle, or complexes thereof.

Formulations of the pharmaceutical compositions described herein may be prepared by any method known or hereafter developed in the art of pharmacology. In general, such preparatory methods include the step of bringing the active ingredient into association with an excipient and/or one or more other accessory ingredients, and then, if necessary and/or desirable, dividing, shaping and/or packaging the product into a desired single- or multi-dose unit.

Relative amounts of the active ingredient, the pharmaceutically acceptable excipient, and/or any additional ingredients in a pharmaceutical composition in accordance with the disclosure will vary, depending upon the identity, size, and/or condition of the subject treated and further depending upon the route by which the composition is to be administered. By way of example, the composition may include between 0.1% and 100%, e.g., between 0.5% and 70%, between 1% and 30%, between 5% and 80%, or at least 80% (w/w) active ingredient.

The mRNAs of the disclosure or a vector encoding the mRNA can be formulated using one or more excipients to: (1) increase stability: (2) increase cell transfection; (3) permit the sustained or delayed release (e.g., from a depot formulation of the mRNA); (4) alter the biodistribution (e.g., target the mRNA to specific tissues or cell types); (5) increase the translation of a polypeptide encoded by the mRNA in vivo; and/or (6) alter the release profile of a polypeptide encoded by the mRNA in vivo. In addition to traditional excipients such as any and all solvents, dispersion media, diluents, or other liquid vehicles, dispersion or suspension aids, surface active agents, isotonic agents, thickening or emulsifying agents, preservatives, excipients of the present disclosure can include, without limitation, lipidoids, liposomes, lipid nanoparticles (e.g., liposomes and micelles), polymers, lipoplexes, core-shell nanoparticles, peptides, proteins, carbohydrates, cells transfected with mRNAs (e.g., for transplantation into a subject), hyaluronidase, nanoparticle mimics and combinations thereof. Accordingly, the formulations of the disclosure can include one or more excipients, each in an amount that together increases the stability of the mRNA, increases cell transfection by the mRNA, increases the expression of a polypeptide encoded by the mRNA, and/or alters the release profile of a mRNA-encoded polypeptide. Further, the mRNAs of the present disclosure may be formulated using self-assembled nucleic acid nanoparticles.

Various excipients for formulating pharmaceutical compositions and techniques for preparing the composition are known in the art (see Remington: The Science and Practice of Pharmacy, 21st Edition, A. R. Gennaro, Lippincott, Williams & Wilkins, Baltimore, Md., 2006; incorporated herein by reference in its entirety). The use of a conventional excipient medium may be contemplated within the scope of the present disclosure, except insofar as any conventional excipient medium may be incompatible with a substance or its derivatives, such as by producing any undesirable biological effect or otherwise interacting in a deleterious manner with any other component(s) of the pharmaceutical composition. Excipients may include, for example: antiadherents, antioxidants, binders, coatings, compression aids, disintegrants, dyes (colors), emollients, emulsifiers, fillers (diluents), film formers or coatings, glidants (flow enhancers), lubricants, preservatives, printing inks, sorbents, suspensing or dispersing agents, sweeteners, and waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol.

In some embodiments, the formulations described herein may include at least one pharmaceutically acceptable salt. Examples of pharmaceutically acceptable salts that may be included in a formulation of the disclosure include, but are not limited to, acid addition salts, alkali or alkaline earth metal salts, mineral or organic acid salts of basic residues such as amines; alkali or organic salts of acidic residues such as carboxylic acids; and the like. Representative acid addition salts include acetate, acetic acid, adipate, alginate, ascorbate, aspartate, benzenesulfonate, benzene sulfonic acid, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like.

In some embodiments, the formulations described herein may contain at least one type of polynucleotide. As a non-limiting example, the formulations may contain 1, 2, 3, 4, 5 or more than 5 mRNAs described herein. In some embodiments, the formulations described herein may contain at least one mRNA encoding a polypeptide and at least one nucleic acid sequence such as, but not limited to, an siRNA, an shRNA, a snoRNA, and an miRNA.

Liquid dosage forms for e.g., parenteral administration include, but are not limited to, pharmaceutically acceptable emulsions, microemulsions, nanoemulsions, solutions, suspensions, syrups, and/or elixirs. In addition to active ingredients, liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, oral compositions can include adjuvants such as wetting agents, emulsifying and/or suspending agents. In certain embodiments for parenteral administration, compositions are mixed with solubilizing agents such as CREMAPHOR®, alcohols, oils, modified oils, glycols, polysorbates, cyclodextrins, polymers, and/or combinations thereof.

Injectable preparations, for example, sterile injectable aqueous or oleaginous suspensions may be formulated according to the known art using suitable dispersing agents, wetting agents, and/or suspending agents. Sterile injectable preparations may be sterile injectable solutions, suspensions, and/or emulsions in nontoxic parenterally acceptable diluents and/or solvents, for example, as a solution in 1,3-butanediol. Among the acceptable vehicles and solvents that may be employed are water, Ringer's solution, U.S.P., and isotonic sodium chloride solution. Sterile, fixed oils are conventionally employed as a solvent or suspending medium. For this purpose any bland fixed oil can be employed including synthetic mono- or diglycerides. Fatty acids such as oleic acid can be used in the preparation of injectables. Injectable formulations can be sterilized, for example, by filtration through a bacterial-retaining filter, and/or by incorporating sterilizing agents in the form of sterile solid compositions which can be dissolved or dispersed in sterile water or other sterile injectable medium prior to use.

In some embodiments, pharmaceutical compositions including at least one mRNA described herein are administered to mammals (e.g., humans). Although the descriptions of pharmaceutical compositions provided herein are principally directed to pharmaceutical compositions which are suitable for administration to humans, it will be understood by the skilled artisan that such compositions are generally suitable for administration to any other animal, e.g., to a non-human mammal. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals, including commercially relevant mammals such as cattle, pigs, horses, sheep, cats, dogs, mice, and/or rats; and/or birds, including commercially relevant birds such as poultry, chickens, ducks, geese, and/or turkeys. In particular embodiments, a subject is provided with two or more mRNAs described herein. In particular embodiments, the first and second mRNAs are provided to the subject at the same time or at different times, e.g., sequentially. In particular embodiments, the first and second mRNAs are provided to the subject in the same pharmaceutical composition or formulation, e.g., to facilitate uptake of both mRNAs by the same cells.

A pharmaceutical composition including one or more mRNAs or a vector encoding the mRNA of the disclosure may be administered to a subject by any suitable route. In some embodiments, compositions of the disclosure are administered by one or more of a variety of routes, including parenteral (e.g., subcutaneous, intracutaneous, intravenous, intraperitoneal, intramuscular, intraarticular, intraarterial, intrasynovial, intrasternal, intrathecal, intralesional, or intracranial injection, as well as any suitable infusion technique), oral, trans- or intra-dermal, interdermal, rectal, intravaginal, topical (e.g. by powders, ointments, creams, gels, lotions, and/or drops), mucosal, nasal, buccal, enteral, vitreal, intratumoral, sublingual, intranasal; by intratracheal instillation, bronchial instillation, and/or inhalation; as an oral spray and/or powder, nasal spray, and/or aerosol, and/or through a portal vein catheter. In some embodiments, a composition may be administered intravenously, intramuscularly, intradermally, intra-arterially, intratumorally, subcutaneously, or by inhalation. In some embodiments, a composition is administered intramuscularly. However, the present disclosure encompasses the delivery of compositions of the disclosure by any appropriate route taking into consideration likely advances in the sciences of drug delivery. In general, the most appropriate route of administration will depend upon a variety of factors including the nature of the pharmaceutical composition including one or more mRNAs (e.g., its stability in various bodily environments such as the bloodstream and gastrointestinal tract), and the condition of the patient (e.g., whether the patient is able to tolerate particular routes of administration).

In certain embodiments, compositions of the disclosure may be administered at dosage levels sufficient to deliver from about 0.0001 mg/kg to about 10 mg/kg, from about 0.001 mg/kg to about 10 mg/kg, from about 0.005 mg/kg to about 10 mg/kg, from about 0.01 mg/kg to about 10 mg/kg, from about 0.1 mg/kg to about 10 mg/kg, from about 1 mg/kg to about 10 mg/kg, from about 2 mg/kg to about 10 mg/kg, from about 5 mg/kg to about 10 mg/kg, from about 0.0001 mg/kg to about 5 mg/kg, from about 0.001 mg/kg to about 5 mg/kg, from about 0.005 mg/kg to about 5 mg/kg, from about 0.01 mg/kg to about 5 mg/kg, from about 0.1 mg/kg to about 10 mg/kg, from about 1 mg/kg to about 5 mg/kg. from about 2 mg/kg to about 5 mg/kg, from about 0.0001 mg/kg to about 1 mg/kg. from about 0.001 mg/kg to about 1 mg/kg. from about 0.005 mg/kg to about 1 mg/kg, from about 0.01 mg/kg to about 1 mg/kg, or from about 0.1 mg/kg to about 1 mg/kg in a given dose, where a dose of 1 mg/kg provides 1 mg of mRNA or nanoparticle per 1 kg of subject body weight. In particular embodiments, a dose of about 0.005 mg/kg to about 5 mg/kg of mRNA or nanoparticle of the disclosure may be administrated.

A dose may be administered one or more times per day, in the same or a different amount, to obtain a desired level of mRNA expression and/or effect (e.g., a therapeutic effect). The desired dosage may be delivered, for example, three times a day, two times a day, once a day, every other day, every third day, every week, every two weeks, every three weeks, or every four weeks. In certain embodiments, the desired dosage may be delivered using multiple administrations (e.g., two, three, four, five, six, seven, eight, nine, ten, eleven, twelve, thirteen, fourteen, or more administrations), In some embodiments, a single dose may be administered, for example, prior to or after a surgical procedure or in the instance of an acute disease, disorder, or condition. The specific therapeutically effective, prophylactically effective, or otherwise appropriate dose level for any particular patient will depend upon a variety of factors including the severity and identify of a disorder being treated, if any; the one or more mRNAs employed; the specific composition employed; the age, body weight, general health, sex, and diet of the patient; the time of administration, route of administration, and rate of excretion of the specific pharmaceutical composition employed; the duration of the treatment; drugs used in combination or coincidental with the specific pharmaceutical composition employed; and like factors well known in the medical arts.

An mRNA or composition (e.g., a pharmaceutical composition) or a vector encoding the mRNA of the disclosure may be administered by any route which results in a therapeutically effective outcome. These include, but are not limited, to intradermal, intramuscular, intranasal, and/or subcutaneous administration. The present disclosure provides methods comprising administering RNA compositions and lipid nanoparticles of the disclosure to a subject in need thereof. The exact amount will vary from subject to subject, depending on the species, age, and general condition of the subject, the severity of the disease, the particular composition, its mode of administration, its mode of activity, and the like. RNA compositions and lipid nanoparticles of the disclosure are typically formulated in dosage unit form for ease of administration and uniformity of dosage. It will be understood, however, that the total daily usage of RNA (e.g., mRNA) or vector containing compositions may be decided by the attending physician within the scope of sound medical judgment. The specific therapeutically effective, prophylactically effective, or appropriate imaging dose level for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

The effective amount of an RNA composition, a vector or lipid nanoparticle of the disclosure, as provided herein, may be as low as 10 pg, administered for example as a single dose or as two 5 μg doses. In some embodiments, the effective amount is a total dose of 10 μg-300 μg, For example, the effective amount may be a total dose of 10 μg, 20 μg, 25 μg, 30 μg, 35 μg, 40 μg, 45 μg, 50 μg, 55 μg, 60 μg, 65 μg, 70 μg, 75 μg, 80 μg, 85 μg, 90 μg, 95 μg, 100 μg, 110 μg, 120 μg, 130 μg, 140 μg, 150 μg, 160 μg, 170 μg, 180 μg, 190 μg or 200 μg, 210 μg, 220 μg, 230 μg, 240 μg, 250 μg, 260 μg, 270 μg, 280 μg, 290 μg or 300 μg. In some embodiments, the effective amount is a total dose of 10 μg-300 μg. In some embodiments, the effective amount is a total dose of 30 μg-100 μg or 50 μg-200 μg.

In some embodiments, RNA (e.g., mRNA) compositions, a vector and lipid nanoparticles may be administered at dosage levels sufficient to deliver 0.0001 mg/kg to 100 mg/kg, 0.001 mg/kg to 0.05 mg/kg. 0.005 mg/kg to 0.05 mg/kg, 0.001 mg/kg to 0.005 mg/kg, 0.05 mg/kg to 0.5 mg/kg, 0.01 mg/kg to 50 mg/kg. 0.1 mg/kg to 40 mg/kg. 0.5 mg/kg to 30 mg/kg, 0.01 mg/kg to 10 mg/kg, 0.1 mg/kg to 10 mg/kg, or 1 mg/kg to 25 mg/kg, of subject body weight per day, one or more times a day, per week, per month, etc. to obtain the desired therapeutic, diagnostic, prophylactic, or imaging effect (see e.g., the range of unit doses described in International Publication No. WO2013078199, herein incorporated by reference in its entirety). The desired dosage may be delivered three times a day, two times a day, once a day, every other day, every third day, every week, every two weeks, every three weeks, every four weeks, every 2 months, every three months, every 6 months, etc. In certain embodiments, the desired dosage may be delivered using multiple administrations (e.g., two, three, four, five, six, seven, eight, nine, ten, eleven, twelve, thirteen, fourteen, or more administrations). When multiple administrations are employed, split dosing regimens such as those described herein may be used. In exemplary embodiments, RNA (e.g., mRNA) compositions may be administered at dosage levels sufficient to deliver 0.0005 mg/kg to 0.01 mg/kg, e.g., about 0.0005 mg/kg to about 0.0075 mg/kg, e.g., about 0.0005 mg/kg, about 0.001 mg/kg, about 0.002 mg/kg, about 0.003 mg/kg, about 0.004 mg/kg or about 0.005 mg/kg.

In some embodiments, RNA (e.g., mRNA) or vector compositions may be administered once or twice (or more) at dosage levels sufficient to deliver 0.025 mg/kg to 0.250 mg/kg, 0.025 mg/kg to 0.500 mg/kg, 0.025 mg/kg to 0.750 mg/kg, or 0.025 mg/kg to 1.0 mg/kg.

In one embodiment, the LNP may include MC3.

### Exemplary Disorders or Diseases for Use with the Compositions

The compositions may be employed to prevent, inhibit or treat a variety of disorders or diseases associated with a deficiency in (or absence of) a protein or an aberrant protein (e.g., with low or no activity or excessive or unregulated activity) (see Table 1 for a list of monogenic disorders). Genes that may be employed include but are not limited to those that prevent, inhibit or treat hemophilia, anemia or other blood disorders, cancer, cardiovascular disease, lysosomal storage diseases, musculoskeletal diseases, neurodegenerative diseases, respiratory disease, and the like. Exemplary genes are shown in Table 2.

TABLE 1

| | |
|---|---|
| Monogenic disorders | Cancer |
| Adrenoleukodystrophy | Gynaecological - breast, ovary, cervix, |
| | vulva |
| α-1 antitrypsin deficiency | Nervous system - glioblastoma, |
| | leptomeningeal carcinomatosis, glioma, |
| | astrocytoma, neuroblastoma, |
| | retinoblastoma |
| Becker muscular dystrophy | Gastrointestinal - colon, colorectal, liver |
| | metastases, post-hepatitis liver cancer, |
| | pancreas, gall bladder |
| β-thalassaemia | Genitourinary - prostate, renal, bladder, |
| | anogenital neoplasia |
| Canavan disease | Skin - melanoma (malignant/metastatic) |
| Chronic granulomatous disease | Head and neck - nasopharyngeal |
| | carcinoma, squamous cell carcinoma, |
| | oesophageal cancer |
| Cystic fibrosis | Lung - adenocarcinoma, small |
| | cell/nonsmall cell, mesothelioma |
| Duchenne muscular dystrophy | Haematological - leukaemia, |
| | lymphoma, multiple myeloma |
| Fabry disease | Sarcoma |
| Familial adenomatous polyposis | Germ cell |
| Familial hypercholesterolaemia | Li-Fraumeni syndrome |
| Fanconi anaemia | Thyroid |
| Galactosialidosis | Neurological diseases |
| Gaucher's disease | Alzheimer's disease |
| Gyrate atrophy | Amyotrophic lateral sclerosis |
| Haemophilia A and B | Carpal tunnel syndrome |
| Hurler syndrome | Cubital tunnel syndrome |
| Hunter syndrome | Diabetic neuropathy |
| Huntington's chorea | Epilepsy |
| Junctional epidermolysis bullosa | Multiple sclerosis |
| Late infantile neuronal ceroid | Myasthenia gravis |
| lipofuscinosis | |
| Leukocyte adherence deficiency | Parkinson's disease |
| Limb girdle muscular dystrophy | Peripheral neuropathy |
| Lipoprotein lipase deficiency | Pain |
| Mucopolysaccharidosis type VII | Ocular diseases |
| Ornithine transcarbamylase | Age-related macular degeneration |
| deficiency | |
| Pompe disease | Diabetic macular edema |
| Purine nucleoside phosphorylase | Glaucoma |
| deficiency | |
| Recessive dystrophic | Retinitis pigmentosa |
| epidermolysis bullosa | |
| Sickle cell disease | Superficial corneal opacity |
| Severe combined | Choroideraemia |
| immunodeficiency | |
| Tay Sachs disease | Leber congenital amaurosis |
| Wiskott-Aldrich syndrome | Inflammatory diseases |
| Cardiovascular disease | Arthritis (rheumatoid, inflammatory, |
| | degenerative) |
| Anaemia of end stage renal | Degenerative joint disease |
| disease | |
| Angina pectoris (stable, | Degenerative joint disease |
| unstable, refractory) | |
| Coronary artery stenosis | Ulcerative colitis |
| Critical limb ischaemia | Severe inflammatory disease of |
| | the rectum |
| Heart failure | Other diseases |
| Intermittent claudication | Chronic renal disease |
| Myocardial ischaemia | Erectile dysfunction |
| Peripheral vascular disease | Detrusor overactivity |
| Pulmonary hypertension | Parotid salivary hypofunction |
| Tangier disease | |
| (hypoalphalipoproteinemia) | |
| Venous ulcers | Oral mucositis |
| Infectious disease | Fractures |
| Adenovirus infection | Type I diabetes |
| Cytomegalovirus infection | Diabetic ulcer/foot ulcer |
| Epstein-Barr virus | Graft versus host disease/transplant |
| | patients |
| Hepatitis B and C | |
| HIV/AIDS | |
| Influenza | |
| Japanese encephalitis | |
| Malaria | |
| Paediatric respiratory disease | |
| Respiratory syncytial virus | |
| Tetanus | |
| Tuberculosis | |

TABLE 2

| Gene Symbol | Protein name | Related Diseases |
|---|---|---|
| ABCA1 | ATP-binding cassette | Tangier disease |
| | transporter ABCA1 | |
| BCL2L11 | BCL2-like 11 (apoptosis | Cancer, e.g., human |
| | facilitator) | T-cell acute |
| | | lymphoblastic |
| | | leukemia and |
| | | lymphoma |
| BRCA1 | breast cancer 1, early | Cancer, e.g., breast |
| | onset | cancer, pancreatic |
| | | cancer |
| F8 | coagulation factor VIII, | Hemophilia |
| | procoagulant component | |
| FLI1 | Friend leukemia virus | cancer, e.g., Ewing's |
| | integration 1 | sarcoma, and |
| | | myelodysplasia |
| FMR1 | fragile X mental | Fragile X syndrome |
| | retardation 1 | and premature |
| | | ovarian failure |
| FNDC5 | fibronectin type III | Obesity, Type 2 |
| | domain containing 5 | Diabetes |
| GCK | glucokinase (hexokinase | Obesity, Type 2 |
| | 4) | Diabetes, and |
| | | Hyperinsulinemic |
| | | hypoglycemia |
| GLP1R | glucagon-like peptide 1 | Type 2 Diabetes |
| | receptor | |
| GRN | granulin | autoimmune, |
| | | inflammatory, |
| | | dementia/CNS |
| | | disease, cancer, e.g., |
| | | hepatic cancer |
| HAMP | hepcidin antimicrobial | hemochromatosis, |
| | peptide | thalassemia |
| HPRT1 | hypoxanthine | Lesch-Nyhan disease |
| | phosphoribosyltransferase | and HPRT-related |
| | 1 | gout |
| IDO1 | indoleamine 2,3- | autoimmune and |
| | dioxygenase 1 | inflammatory |
| | | diseases |
| IGF1 | insulin-like growth factor 1 | metabolic disease, |
| | (somatomedin C) | delayed growth, |
| | | cancer |
| IL10 | interleukin 10 | Autoimmune and |
| | | inflammatory |
| | | diseases, e.g., |
| | | graft vs. |
| | | host disease and |
| | | rheumatoid arthritis |
| LDLR | low density lipoprotein | dyslipidemias, |
| | receptor | atherosclerosis , and |
| | | hypercholesterolemia |
| NANOG | Nanog homeobox | tissue regeneration |
| PTGS2 | prostaglandin- | inflammation, cancer, |
| | endoperoxide synthase 2 | infectious disease |
| | (prostaglandin G/H | |
| | synthase and | |
| | cyclooxygenase) | |
| RB1 | retinoblastoma 1 | cancer, e.g., bladder |
| | | cancer, |
| | | osteosarcoma, |
| | | retinoblastoma, small |
| | | cell lung cancer |
| SCARB1 | Scavenger receptor class | Coronary artery |
| | B type 1 | disease, |
| | | atherosclerosis |
| SERPINF1 | serpin peptidase inhibitor, | cancer, choroidal |
| | Glade F (alpha-2 | neovascularization, |
| | antiplasmin, pigment | cardiovascular |
| | epithelium derived | disease, diabetes, |
| | factor), member 1 | and osteogenesis |
| | | imperfecta |
| SIRT1 | sirtuin 1 | Metabolic disease, |
| | | aging |
| SIRT6 | sirtuin 6 | antioxidative |
| | | pathway, anti-NFKB |
| SMAD7 | SMAD family member 7 | Acute kidney injury |
| | | (anti-TGFb), |
| | | colorectal cancer |
| ST7 | suppression of | cancer, e.g., myeloid |
| | tumorigenicity 7 | cancer, head and neck |
| | | squamous cell |
| | | carcinomas, breast |
| | | cancer, colon |
| | | carcinoma, and |
| | | prostate cancer |
| STAT3 | signal transducer and | tissue regeneration |
| | activator of transcription 3 | and Hyper-IgE |
| | (acute-phase response | recurrent infection |
| | factor) | syndrome |
| CFTR | Cystic fibrosis | Cystic fibrosis (CF) |
| | transmembrane | and congenital |
| | conductance regulator | bilateral absence of |
| | | vas deferens |
| | | (CBAVD) |
| PAH | Phenylalanine | Phenylketonuria |
| | hydroxylase | (PKU) |
| CEP290 | Centrosomal protein of | Leber's congenital |
| | 290 kDa | amaurosis (LCA), |
| | | Bardet-Biedl |
| | | syndrome (BBS), |
| | | Joubert syndrome, |
| | | Meckel syndrome, |
| | | Sior-Loken syndrome |
| CD274 | cluster of differentiation | Autoimmune disease, |
| (also | 274 (also known as | transplant rejection, |
| known as | Programmed cell death 1 | allergies or asthma |
| PD-L1) | ligand 1) | |
| ADIPOQ | adiponectin, C1Q and | Obesity and obesity- |
| | collagen domain | linked diseases (e.g., |
| | containing (also known as | hypertension, |
| | adiponectin) | metabolic |
| | | dysfunction, type 2 |
| | | diabetes, |
| | | atherosclerosis, and |
| | | ischemic heart |
| | | disease) |

### Hemophilia-F8. F9, F11, VWF

Hemophilia is a group of hereditary genetic disorders that impair the body's ability to control blood clotting or coagulation, which is used to stop bleeding when a blood vessel is broken. Like most recessive sex-linked, X chromosome disorders, hemophilia is more likely to occur in males than females. For example, Hemophilia A (clotting factor Vill deficiency), the most common form of the disorder, is present in about 1 in 5,000-10,000 male births. Hemophilia B (factor IX deficiency) occurs in around 1 in about 20,000-34,000 male births, Hemophilia lowers blood plasma clotting factor levels of the coagulation factors, e.g., F8, needed for a normal clotting process. Thus, when a blood vessel is injured, a temporary scab does form, but the missing coagulation factors prevent fibrin formation, which is necessary to maintain the blood clot. F8, for example, encodes Factor VIII (FVIII), an essential blood clotting protein. Factor VIII participates in blood coagulation; it is a cofactor for factor IXa which, in the presence of Ca+2 and phospholipids forms a complex that converts factor X to the activated form Xa.

Aspects of the disclosure herein provide methods and compositions that are useful for upregulating F8 for the treatment and/or prevention of diseases associated with reduced F8 expression or function such as hemophilia. Aspects of the disclosure herein provide methods and compositions that are useful for upregulating F9 for the treatment and/or prevention of diseases associated with reduced F9 expression or function such as hemophilia. Aspects of the disclosure herein provide methods and compositions that are useful for upregulating F11 for the treatment and/or prevention of diseases associated with reduced F11 expression or function such as hemophilia. Aspects of the disclosure herein provide methods and compositions that are useful for upregulating VWF for the treatment and/or prevention of diseases associated with reduced VFW expression or function such as Von Willebrand's Disease

Thus, in one embodiment, the compositions may be employed to prevent, inhibit or treat hemophilia including but not limited to hemophilia A, characterized by low levels of or the absence of factor 8 (Also called FVIII or factor VIII deficiency), hemophilia B, characterized by low levels of or the absence of factor 9 (Also called FIX or factor IX deficiency), hemophilia C, characterized by low levels of or the absence of factor 11 (Also called FXI or factor XI deficiency), or Von Willebrands Disease, characterized by a deficiency of a blood clotting protein Von Willebrand factor.

### Lysosomal Storage Diseases

In one embodiment, the compositions may be employed to prevent, inhibit or treat a lysosomal storage disease. Lysosomal storage diseases include, but are not limited to, mucopolysaccharidosis (MPS) diseases, for instance, mucopolysaccharidosis type I, e.g., Hurler syndrome and the variants Scheie syndrome and Hurler-Scheie syndrome (a deficiency in alpha-L-iduronidase); Hunter syndrome (a deficiency of iduronate-2-sulfatase); mucopolysaccharidosis type III, e.g., Sanfilippo syndrome (A, B, C or D; a deficiency of heparan sulfate sulfatase, N-acetyl-alpha-D-glucosaminidase, acetyl CoÅ:alpha-glucosaminide N-acetyl transferase or N-acetylglucosamine-6-sulfate sulfatase); mucopolysaccharidosis type IV e.g., mucopolysaccharidosis type IV, e.g., Morquio syndrome (a deficiency of galactosamine-6-sulfate sulfatase or beta-galactosidase); mucopolysaccharidosis type VI, e.g., Maroteaux-Lamy syndrome (a deficiency of arylsulfatase B); mucopolysaccharidosis type II; mucopolysaccharidosis type III (A, B, C or D; a deficiency of heparan sulfate sulfatase, N-acetyl-alpha-D-glucosaminidase, acetyl CoA:alpha-glucosaminide N-acetyl transferase or N-acetylglucosamine-6-sulfate sulfatase); mucopolysaccharidosis type IV (A or B; a deficiency of galactosamine-6-sulfatase and beta-galatacosidase); mucopolysaccharidosis type VI (a deficiency of arylsulfatase B); mucopolysaccharidosis type VII (a deficiency in beta-glucuronidase); mucopolysaccharidosis type VIII (a deficiency of glucosamine-6-sulfate sulfatase); mucopolysaccharidosis type IX (a deficiency of hyaluronidase); Tay-Sachs disease (a deficiency in alpha subunit of beta-hexosaminidase); Sandhoff disease (a deficiency in both alpha and beta subunit of beta-hexosaminidase); GM1 gangliosidosis (type I or type II); Fabry disease (a deficiency in alpha galactosidase); metachromatic leukodystrophy (a deficiency of aryl sulfatase A); Pompe disease (a deficiency of acid maltase); fucosidosis (a deficiency of fucosidase); alpha-mannosidosis (a deficiency of alpha-mannosidase); beta-mannosidosis (a deficiency of beta-mannosidase), ceroid lipofuscinosis, and Gaucher disease (types I, Il and Ill; a deficiency in glucocerebrosidase), as well as disorders such as Hermansky-Pudlak syndrome; Amaurotic idiocy; Tangier disease; aspartylglucosaminuria; congenital disorder of glycosylation, type la; Chediak-Higashi syndrome; macular dystrophy, comeal, 1; cystinosis, nephropathic; Fanconi-Bickel syndrome; Farber lipogranulomatosis; fibromatosis; geleophysic dysplasia; glycogen storage disease l; glycogen storage disease lb; glycogen storage disease Ic; glycogen storage disease Ill; glycogen storage disease IV; glycogen storage disease V; glycogen storage disease VI; glycogen storage disease VII; glycogen storage disease 0; immunoosseous dysplasia, Schimke type; lipidosis; lipase b; mucolipidosis Il, including the variant form; mucolipidosis IV; neuraminidase deficiency with beta-galactosidase deficiency; mucolipidosis I; Niemann-Pick disease (a deficiency of sphingomyelinase); Niemann-Pick disease without sphingomyelinase deficiency (a deficiency of a npc1 gene encoding a cholesterol metabolizing enzyme); Refsum disease; Sea-blue histiocyte disease; infantile sialic acid storage disorder; sialuria; multiple sulfatase deficiency; triglyceride storage disease with impaired long-chain fatty acid oxidation; Winchester disease; Wolman disease (a deficiency of cholesterol ester hydrolase); Deoxyribonuclease I-like 1 disorder; arylsulfatase E disorder; ATPase, H+transporting, lysosomal, subunit 1 disorder; glycogen storage disease Ilb; Ras-associated protein rab9 disorder; chondrodysplasia punctata 1, X-linked recessive disorder; glycogen storage disease VIII; lysosome-associated membrane protein 2 disorder; Menkes syndrome; congenital disorder of glycosylation, type Ic; and sialuria.

### Cancer-SERPINF1, BCL2L11, BRCA1, RB1, ST7

In one embodiment, the compositions may be employed to prevent, inhibit or treat cancer. Cancer is a broad group of various diseases, all involving unregulated cell growth. In cancer, cells divide and grow uncontrollably, forming malignant tumors, and invade nearby parts of the body. Several genes, many classified as tumor suppressors, are down-regulated during cancer progression, e.g., SERPINF1, BCL2L11, BRCA1, RB1, and ST7, and have roles in inhibiting genomic instability, metabolic processes, immune response, cell growth/cell cycle progression, migration, and/or survival. These cellular processes are important for blocking tumor progression. SERPINF1 encodes an anti-angiogenic factor. BCL2L11 encodes an apoptosis facilitator. BRCA1 encodes a RING finger protein involved in DNA damage repair. RB1 prevents excessive cell growth by inhibiting cell cycle progression until a cell is ready to divide. ST7 suppresses tumor growth in mouse models and is involved in regulation of genes involved in differentiation. Aspects of the disclosure herein provide methods and compositions that are useful for upregulating SERPINF1, BCL2L11, BRCA1, RB1, and ST7 for the treatment and/or prevention of diseases associated with reduced SERPINF1, BCL2L11, BRCA1, RB1, and ST7 expression or function such as cancer. For example, aspects of the invention disclosed herein provide methods and compositions that are useful for upregulating BCL2L11 for the treatment or prevention of human T-cell acute lymphoblastic leukemia and lymphoma. In another example, aspects of the invention disclosed herein provide methods and compositions that are useful for upregulating BRCA1 for the treatment or prevention of breast cancer or pancreatic cancer. In another example, aspects of the invention disclosed herein provide methods and compositions that are useful for upregulating RB1 for the treatment or prevention of bladder cancer, osteosarcoma, retinoblastoma, or small cell lung cancer. In another example, aspects of the invention disclosed herein provide methods and compositions that are useful for upregulating ST7 for the treatment or prevention of myeloid cancer, head and neck squamous cell carcinomas, breast cancer, colon carcinoma, or prostate cancer.

Examples of cancer include but are not limited to leukemias, lymphomas, myelomas, carcinomas, metastatic carcinomas, sarcomas, adenomas, nervous system cancers and genito-urinary cancers. In some embodiments, the cancer is adult and pediatric acute lymphoblastic leukemia, acute myeloid leukemia, adrenocortical carcinoma, AIDS-related cancers, anal cancer, cancer of the appendix, astrocytoma, basal cell carcinoma, bile duct cancer, bladder cancer, bone cancer, osteosarcoma, fibrous histiocytoma, brain cancer, brain stem glioma, cerebellar astrocytoma, malignant glioma, ependymoma, medulloblastoma, supratentorial primitive neuroectodermal tumors, hypothalamic glioma, breast cancer, male breast cancer, bronchial adenomas, Burkitt lymphoma, carcinoid tumor, carcinoma of unknown origin, central nervous system lymphoma, cerebellar astrocytoma, malignant glioma, cervical cancer, childhood cancers, chronic lymphocytic leukemia, chronic myelogenous leukemia, chronic myeloproliferative disorders, colorectal cancer, cutaneous T-cell lymphoma, endometrial cancer, ependymoma, esophageal cancer, Ewing family tumors, extracranial germ cell tumor, extragonadal germ cell tumor, extrahepatic bile duct cancer, intraocular melanoma, retinoblastoma, gallbladder cancer, gastric cancer, gastrointestinal stromal tumor, ovarian germ cell tumor, gestational trophoblastic tumor, glioma, hairy cell leukemia, head and neck cancer, hepatocellular cancer, Hodgkin lymphoma, non-Hodgkin lymphoma, hypopharyngeal cancer, hypothalamic and visual pathway glioma, intraocular melanoma, islet cell tumors, Kaposi sarcoma, kidney cancer, renal cell cancer, laryngeal cancer, lip and oral cavity cancer, small cell lung cancer, non-small cell lung cancer, primary central nervous system lymphoma, Waldenstrom macroglobulinemia, malignant fibrous histiocytoma, medulloblastoma, melanoma, Merkel cell carcinoma, malignant mesothelioma, squamous neck cancer, multiple endocrine neoplasia syndrome, multiple myeloma, mycosis fungoides, myelodysplastic syndromes, myeloproliferative disorders, chronic myeloproliferative disorders, nasal cavity and paranasal sinus cancer, nasopharyngeal cancer, neuroblastoma, oropharyngeal cancer, ovarian cancer, pancreatic cancer, parathyroid cancer, penile cancer, pharyngeal cancer, pheochromocytoma, pineoblastoma and supratentorial primitive neuroectodermal tumors, pituitary cancer, plasma cell neoplasms, pleuropulmonary blastoma, prostate cancer, rectal cancer, rhabdomyosarcoma, salivary gland cancer, soft tissue sarcoma, uterine sarcoma, Sezary syndrome, non-melanoma skin cancer, small intestine cancer, squamous cell carcinoma, squamous neck cancer, supratentorial primitive neuroectodermal tumors, testicular cancer, throat cancer, thymoma and thymic carcinoma, thyroid cancer, transitional cell cancer, trophoblastic tumors, urethral cancer, uterine cancer, uterine sarcoma, vaginal cancer, vulvar cancer, or Wilms tumor.

### Fragile X Syndrome—FMR1

Fragile X syndrome (FXS) (also known as Martin-Bell syndrome, or Escalante's syndrome) is a genetic syndrome that is the most commonly known single-gene cause of autism and the most common inherited cause of intellectual disability. It results in a spectrum of intellectual disability ranging from mild to severe as well as physical characteristics such as an elongated face, large or protruding ears, and larger testes (macroorchidism), behavioral characteristics such as stereotypical movements (e.g. hand-flapping), and social anxiety. Fragile X syndrome is associated with the expansion of the CGG trinucleotide repeat affecting the Fragile X mental retardation 1 (FMR1) gene on the X chromosome, resulting reduced expression of the X mental retardation protein (FMRP), which is required for normal neural development. Aspects of the invention disclosed herein provide methods and compositions that are useful for upregulating FMR1 for the treatment and/or prevention of diseases associated with reduced FMR1 expression or function such as Fragile X syndrome.

### Premature Ovarian Failure—FMR1

Premature Ovarian Failure (POF), also known as premature ovarian insufficiency, primary ovarian insufficiency, premature menopause, or hypergonadotropic hypogonadism, is the loss of function of the ovaries before age 40. POF can be associated mutations in the Fragile X mental retardation 1 (FMR1) gene on the X chromosome, resulting reduced expression of the X mental retardation protein (FMRP). Aspects of the disclosure herein provide methods and compositions that are useful for upregulating FMR1 for the treatment and/or prevention of diseases associated with reduced FMR1 expression or function such as Premature Ovarian Failure.

### Obesity—FNDC5, GCK, ADIPOQ

Obesity is a medical condition in which excess body fat has accumulated to the extent that it may have an adverse effect on health, leading to reduced life expectancy and/or increased health problems. A person is considered obese when his or her weight is 20% or more above normal weight. The most common measure of obesity is the body mass index or BMI. A person is considered overweight if his or her BMI is between 25 and 29.9; a person is considered obese if his or her BMI is over 30. Obesity increases the likelihood of various diseases, particularly heart disease, type 2 diabetes, obstructive sleep apnea, certain types of cancer, and osteoarthritis. Obesity is most commonly caused by a combination of excessive food energy intake, lack of physical activity, and genetic susceptibility. Overexpression of FNDC5, fibronectin type II containing 5, has been shown in animal models to reduce body weight in obese mice. GCK, glucokinase (hexokinase 4), phosphorylates glucose to produce glucose-6-phosphate, the first step in most glucose metabolism pathways. Mutations in the GCK gene have been found to be associated with obesity in humans. Aspects of the disclosure herein provide methods and compositions that are useful for upregulating FNDC5 for the treatment and/or prevention of diseases associated with reduced FNDC5 expression or function such as obesity. Aspects of the disclosure herein provide methods and compositions that are useful for upregulating GCK for the treatment and/or prevention of diseases associated with reduced GCK expression or function such as obesity.

Adiponectin, encoded by the ADIPOQ gene, is a hormone that regulates metabolism of lipids and glucose. Adipocytes found in adipose tissue secrete adiponectin into the bloodstream where it self-associates into larger structures by binding of multiple adiponectin trimers to form hexamers and dodecamers. Adiponectin levels are inversely related to the amount of body fat in an individual and positively associated with insulin sensitivity both in healthy subjects and in diabetic patients. Adiponectin has a variety of protective properties against obesity-linked complications, such as hypertension, metabolic dysfunction, type 2 diabetes, atherosclerosis, and ischemic heart disease through its anti-inflammatory and anti-atherogenic properties. Specifically with regard to type 2 diabetes, administration of adiponectin has been accompanied by a reduction in plasma glucose and an increase in insulin sensitivity. Aspects of the disclosure herein provide methods and compositions that are useful for upregulating ADIPOQ for the treatment and/or prevention of diseases associated with reduced ADIPOQ expression or function such as obesity or an obesity-linked disease or disorders such as hypertension, metabolic dysfunction, type 2 diabetes, atherosclerosis, and ischemic heart disease.

### Type 2 Diabetes—FNDC5, GCK, GLP1R, SIRT1, ADIPOQ

Type 2 diabetes (also called Diabetes mellitus type 2 and formally known as adult-onset diabetes) a metabolic disorder that is characterized by high blood glucose in the context of insulin resistance and relative insulin deficiency. Type 2 diabetes makes up about 90% of cases of diabetes with the other 10% due primarily to diabetes mellitus type 1 and gestational diabetes. Obesity is thought to be the primary cause of type 2 diabetes in people who are genetically predisposed to the disease. The prevalence of diabetes has increased dramatically in the last 50 years. As of 2010 there were approximately 285 million people with the disease compared to around 30 million in 1985. Overexpression of FNDC5, fibronectin type Il containing 5, has been shown in animal models to improve their insulin sensitivity. GCK, glucokinase (hexokinase 4), phosphorylates glucose to produce glucose-6-phosphate, the first step in most glucose metabolism pathways. Mutations in the GCK gene are known to be associated with Type 2 Diabetes. Glucagon-like peptide 1 receptor (GLP1R) is known to be expressed in pancreatic beta cells. Activated GLP1R stimulates the adenylyl cyclase pathway which results in increased insulin synthesis and release of insulin. SIRT1 (Sirtuin 1, also known as NAD-dependent deacetylase sirtuin-1) is an enzyme that deacetylates proteins that contribute to cellular regulation. Sirtuin 1 is downregulated in cells that have high insulin resistance and inducing its expression increases insulin sensitivity, suggesting the molecule is associated with improving insulin sensitivity. Aspects of the disclosure herein provide methods and compositions that are useful for upregulating FNDC5 for the treatment and/or prevention of diseases associated with reduced FNDC5 expression or function such as Type 2 Diabetes. Aspects of the disclosure herein provide methods and compositions that are useful for upregulating GCK for the treatment and/or prevention of diseases associated with reduced GCK expression or function such as Type 2 Diabetes. Aspects of the disclosure herein provide methods and compositions that are useful for upregulating GLP1R for the treatment and/or prevention of diseases associated with reduced GLP1R expression or function such as Type 2 Diabetes. Aspects of the disclosure herein provide methods and compositions that are useful for upregulating SIRT1 for the treatment and/or prevention of diseases associated with reduced SIRT1 expression or function such as Type 2 Diabetes. Aspects of the disclosure herein provide methods and compositions that are useful for upregulating ADIPOQ for the treatment and/or prevention of diseases associated with reduced ADIPOQ expression or function such as Type 2 Diabetes.

### Metabolic Disease—IGF1, SIRT1

Inborn errors of metabolism comprise a large class of genetic diseases involving disorders of metabolism. The majority are due to defects of single genes that code for enzymes that facilitate conversion of various substances (substrates) into others (products). In most of the disorders, problems arise due to accumulation of substances which are toxic or interfere with normal function, or to the effects of reduced ability to synthesize essential compounds. Inborn errors of metabolism are now often referred to as congenital metabolic diseases or inherited metabolic diseases. IGF-1, Insulin growth factor-1, is a hormone similar in molecular structure to insulin. IGF-1 plays an important role in childhood growth and continues to have anabolic effects in adults. Reduced IGF-1 and mutations in the IGF-1 gene are associated with metabolic disease. SIRT1 (Sirtuin 1, also known as NAD-dependent deacetylase sirtuin-1) is an enzyme that deacetylates proteins that contribute to cellular regulation. SIRT1 has been shown to de-acetylate and affect the activity of both members of the PGC1-alpha/ERR-alpha complex, which are essential metabolic regulatory transcription factors. Aspects of the disclosure herein provide methods and compositions that are useful for upregulating IGF-1 for the treatment and/or prevention of diseases associated with reduced IGF-1 expression or function such as metabolic disease. Aspects of the disclosure herein provide methods and compositions that are useful for upregulating SIRT1 for the treatment and/or prevention of diseases associated with reduced SIRT1 expression or function such as metabolic disease.

### Aging/Senescence—SIRT1

Senescence is the state or process of aging. Cellular senescence is a phenomenon where isolated cells demonstrate a limited ability to divide in culture, while organismal senescence is the aging of organisms. After a period of near perfect renewal (in humans, between 20 and 35 years of age), organismal senescence/aging is characterised by the declining ability to respond to stress, increasing homeostatic imbalance and increased risk of disease. This currently irreversible series of changes inevitably ends in death. SIRT1 (Sirtuin 1, also known as NAD-dependent deacetylase sirtuin-1) is an enzyme that deacetylates proteins that contribute to cellular regulation. Mice overexpressing SIRT1 present lower levels of DNA damage, decreased expression of the ageing-associated gene p16Ink4a, a better general health and fewer spontaneous carcinomas and sarcomas. Aspects of the disclosure herein provide methods and compositions that are useful for upregulating SIRT1 for the treatment and/or prevention of biological processes associated with reduced SIRT1 expression or function such as aging.

### Autoimmune—GRN, IDO1, CD274

Autoimmune diseases arise from an inappropriate immune response of the body against substances and tissues normally present in the body. In other words, the immune system mistakes some part of the body as a pathogen and attacks its own cells. Autoimmune diseases are classified by corresponding types of hypersensitivity: type Il, type III, or type IV. Examples of autoimmune disease include, but are not limited to, Ankylosing Spondylitis, Autoimmune cardiomyopathy, Autoimmune hemolytic anemia, Autoimmune hepatitis, Autoimmune inner ear disease, immune lymphoproliferative syndrome, Autoimmune peripheral neuropathy, Autoimmune pancreatitis, Autoimmune polyendocrine syndrome, Autoimmune thrombocytopenia purpura, Celiac disease, Cold agglutinin disease, Contact dermatitis, Crohn's disease, Dermatomyositis, Diabetes mellitus type 1, Eosinophilic fasciitis, Gastrointestinal pemphigoid, Goodpasture's syndrome, Graves' disease, Guillain-Barré syndrome, Hashimoto's encephalopathy, Hashimoto's thyroiditis, Idiopathic thrombocytopenia purpura, Lupus erythematosus, Miller-Fisher syndrome, Myasthenia gravis, Pemphigus vulgaris, Pernicious anaemia, Polymyositis, Primary biliary cirrhosis, Psoriasis, Psoriatic arthritis, Relapsing polychondritis, Rheumatoid arthritis, Sjögren's syndrome, Temporal arteritis, Transverse myelitis, Ulcerative colitis, Undifferentiated connective tissue disease, Vasculitis, Vitiligo, and Wegener's granulomatosis. IDO1 encodes indoleamine 2,3-dioxygenase (IDO)-a heme enzyme that catalyzes the first and rate-limiting step in tryptophan catabolismo N-formyl-kynurenine. This enzyme acts on multiple tryptophan substrates including D-tryptophan, L-tryptophan, 5-hydroxy-tryptophan, tryptamine, and serotonin. This enzyme is thought to play a role in a variety of pathophysiological processes such as antimicrobial and antitumor defense, neuropathology, immunoregulation, and antioxidant activity. Increased catabolismof tryptophan by IDO1 suppresses T cell responses in a variety of diseases or states, including autoimmune disorders. GRN encodes a precursor protein called Progranulin, which is then cleaved to form the secreted protein granulin. Granulin regulates cell division, survival, motility and migration. Granulin has roles in cancer, inflammation, host defense, cartilage development and degeneration, and neurological functions, Downregulation of GRN has been shown to increase the onset of autoimmune diseases like rheumatoid arthritis. Aspects of the disclosure herein provide methods and compositions that are useful for upregulating IDO1 for the treatment and/or prevention of diseases associated with reduced IDO1 expression or function such as autoimmune diseases. Aspects of the disclosure herein provide methods and compositions that are useful for upregulating GRN for the treatment and/or prevention of diseases associated with reduced GRN expression or function such as autoimmune diseases.

CD274 (also known as PDL1) is a transmembrane protein containing IgV-like and IgC-like extracellular domains expressed on immune cells and non-hematopoietic cells, and is a ligand for the programmed death receptor (PD-1) expressed on lymphocytes and macrophages. PD-1 and CD274 interactions are essential in maintaining the balance of T-cell activation, tolerance, and immune-mediated tissue damage. CD274 is involved in inhibiting the initial phase of activation and expansion of self-reactive T cells, and restricting self-reactive T-cell effector function and target organ injury. More specifically, activation of PD-1 by CD274 inhibits T-cell proliferation, cytokine production, and cytolytic function by blocking the induction of phosphatidylinositol-3-kinase (PI3K) activity and downstream activation of Akt.

Decreased expression of CD274 results in autoimmunity in animal models. For example, mice deficient for the CD274 receptor, PD-1, developed features of late onset lupus. In another instance, blockade of CD274 activity in a mouse model of Type 1 diabetes resulted in accelerated progression of diabetes. In yet another example, CD274 blockade in an animal model of multiple sclerosis resulted in accelerated disease onset and progression.

Increasing expression of CD274 offers a novel approach for treating diseases related to inappropriate or undesirable activation of the immune system, including in the context of translation rejection, allergies, asthma and autoimmune disorders. Aspects of the disclosure herein provide methods and compositions that are useful for upregulating CD274 for the treatment and/or prevention of diseases associated with reduced CD274 expression or function such as autoimmune disease, transplant rejection, allergies or asthma.

### Inflammation (Chronic Inflammation)—GRN, IDO1, IL 10

Inflammation is part of the complex biological response of vascular tissues to harmful stimuli, such as pathogens, damaged cells, or irritants. Inflammation is a protective attempt by the organism to remove the injurious stimuli and to initiate the healing process. However, chronic inflammation can also lead to a host of diseases, such as hay fever, periodontitis, atherosclerosis, and rheumatoid arthritis. Prolonged inflammation, known as chronic inflammation, leads to a progressive shift in the type of cells present at the site of inflammation and is characterized by simultaneous destruction and healing of the tissue from the inflammatory process. Inflammatory disorders include, but are not limited to, acne vulgaris, asthma, autoimmune diseases, celiac disease, chronic prostatitis, glomerulonephritis, inflammatory bowel diseases, pelvic inflammatory disease, reperfusion injury, rheumatoid arthritis, sarcoidosis, transplantation rejection (graft vs host disease), vasculitis and interstitial cystitis.

GRN encodes a precursor protein called Progranulin, which is then cleaved to form the secreted protein granulin. Granulin regulates cell division, survival, motility and migration. Granulin has roles in cancer, inflammation, host defense, cartilage development and degeneration, and neurological functions. GRN has been shown to alleviate inflammatory arthritis symptoms in mouse models. Indoleamine 2,3-dioxygenase 1 (IDO1; previously referred as IDO or INDO) is the main inducible and rate-limiting enzyme for the catabolismof the amino acid tryptophan through the kynurenine pathway. Increased catabolismof tryptophan by IDO1 suppresses T cell responses in a variety of diseases, such as allograft rejection.

Aspects of the disclosure herein provide methods and compositions that are useful for upregulating GRN for the treatment and/or prevention of diseases associated with reduced GRN expression or function such as chronic inflammation. Aspects of the disclosure herein provide methods and compositions that are useful for upregulating GRN for the treatment and/or prevention of diseases associated with reduced GRN expression or function such as rheumatoid arthritis. Aspects of the disclosure herein provide methods and compositions that are useful for upregulating IDO1 for the treatment and/or prevention of diseases associated with reduced IDO1 expression or function such as chronic inflammation. Aspects of the disclosure herein provide methods and compositions that are useful for upregulating IDO1 for the treatment and/or prevention of diseases associated with reduced IDO1 expression or function such as graft vs. host disease.

IL−10 is capable of inhibiting synthesis of pro-inflammatory cytokines such as IFN-γ, IL-2, IL-3, TNFα and GM-CSF made by cells such as macrophages and regulatory T-cells. It also displays a potent ability to suppress the antigen-presentation capacity of antigen presenting cells. Treatment with IL−10 (e.g. as a recombinant protein given to patients) is currently in clinical trials for Crohn's disease. Genetic variation in the IL−10 pathway modulates severity of acute graft-versus-host disease. Mouse models of arthritis have been shown to have decreased levels of IL-10. Aspects of the disclosure herein provide methods and compositions that are useful for upregulating GRN for the treatment and/or prevention of diseases associated with reduced GRN expression or function such as chronic inflammation.

Aspects of the disclosure herein provide methods and compositions that are useful for upregulating IL−10 for the treatment and/or prevention of diseases associated with reduced IL−10 expression or function such as chronic inflammation. Aspects of the disclosure herein provide methods and compositions that are useful for upregulating IL−10 for the treatment and/or prevention of diseases associated with reduced IL−10 expression or function such as rheumatoid arthritis. Aspects of the disclosure herein provide methods and compositions that are useful for upregulating IL−10 for the treatment and/or prevention of diseases associated with reduced IL−10 expression or function such as graft vs host disease. Aspects of the disclosure herein provide methods and compositions that are useful for upregulating IL−10 for the treatment and/or prevention of diseases associated with reduced IL−10 expression or function such as Crohn's disease.

### Infectious Disease—PTGS2

Infectious diseases, also known as transmissible diseases or communicable diseases comprise clinically evident illness (i.e., characteristic medical signs and/or symptoms of disease) resulting from the infection, presence and growth of pathogenic biological agents in an individual host organism. Infectious pathogens include some viruses, bacteria, fungi, protozoa, multicellular parasites, and aberrant proteins known as prions. A contagious disease is a subset of infectious disease that is especially infective or easily transmitted. Prostaglandin-endoperoxide synthase 2, also known as cyclooxygenase-2 or simply COX-2, is an enzyme that in humans is encoded by the PTGS2 gene. Prostaglandin endoperoxide H synthase, COX 2, converts arachidonic acid (AA) to prostaglandin endoperoxide H2. COX-2 is elevated during inflammation and infection. Aspects of the disclosure herein provide methods and compositions that are useful for upregulating PTGS2 for the treatment and/or prevention of diseases associated with reduced PTGS2 expression or function such as infectious disease.

### CNS Disease—IGF1, GRN

Central nervous system (CNS) disease can affect either the spinal cord (myelopathy) or brain (encephalopathy), both of which are part of the central nervous system. CNS diseases include Encephalitis, Meningitis, Tropical spastic paraparesis, Arachnoid cysts, Amyotrophic lateral sclerosis, Huntington's disease, Alzheimer's disease, Dementia, Locked-in syndrome, Parkinson's disease, Tourette', and Multiple sclerosis. CNS diseases have a variety of causes including Trauma, Infections, Degeneration, Structural defects, Tumors, Autoimmune Disorders, and Stroke. Symptoms range from persistent headache, loss of feeling, memory loss, loss of muscle strength, tremors, seizures, slurred speech, and in some cases, death. IGF-1, Insulin growth factor-1, is a hormone similar in molecular structure to insulin. IGF-I deficiency is associated with neurodegenerative disease and has been shown to improve survival of neurons both in vitro and in vivo. Aspects of the disclosure herein provide methods and compositions that are useful for upregulating IGF1 for the treatment and/or prevention of diseases associated with reduced IGF1 expression or function such as CNS disease.

GRN encodes a precursor protein called Progranulin, which is then cleaved to form the secreted protein granulin. Granulin regulates cell division, survival, motility and migration. Granulin has roles in cancer, inflammation, host defense, cartilage development and degeneration, and neurological functions. Mutations in granulin are associated with dementia. Aspects of the disclosure herein provide methods and compositions that are useful for upregulating GRN for the treatment and/or prevention of diseases associated with reduced GRN expression or function such as CNS disease.

### Hemochromatosis—HAMP

Hemochromatosis is the abnormal accumulation of iron in parenchymal organs, leading to organ toxicity. This is the most common inherited liver disease in Caucasians and the most common autosomal recessive genetic disorder. HAMP (hepcidin antimicrobial peptide) encodes the protein hepcidin, which plays a major role in maintaining iron balance in the body. Hepcidin circulates in the blood and inhibits iron absorption by the small intestine when the body's supply of iron is too high. Hepcidin interacts primarily with other proteins in the intestines, liver, and certain white blood cells to adjust iron absorption and storage. At least eight mutations in the HAMP-gene have been identified that result in reduced levels of hepcidin and hemochromatosis, Aspects of the disclosure herein provide methods and compositions that are useful for upregulating HAMP for the treatment and/or prevention of diseases associated with reduced HAMP expression or function such as hemochromatosis.

### Acute Kidney Injury—SMAD7

Acute kidney injury (AKI), previously called acute renal failure (ARF), is a rapid loss of kidney function. Its causes are numerous and include low blood volume from any cause, exposure to substances harmful to the kidney, and obstruction of the urinary tract. AKI may lead to a number of complications, including metabolic acidosis, high potassium levels, uremia, changes in body fluid balance, and effects to other organ systems. SMAD7 (Mothers against decapentaplegic homolog 7) is a protein that, as its name describes, is a homolog of the Drosophila gene: “Mothers against decapentaplegic”. It belongs to the SMAD family of proteins, which belong to the TGFB superfamily of ligands. Like many other TGFß family members, SMAD7 is involved in cell signaling. It is a TGFβ type 1 receptor antagonist. It blocks TGFB1 and activin associated with the receptor, blocking access to SMAD2. It is an inhibitory SMAD (I-SMAD) and is enhanced by SMURF2. Upon TGF-β treatment, SMAD7 binds to discrete regions of Pellino-1 via distinct regions of the SMAD MH2 domains. The interaction block formation of the IRAK1-mediated IL-1R/TLR signaling complex therefore abrogates NF-κB activity, which subsequently causes reduced expression of pro-inflammatory genes. Overexpression of SMAD7 in the kidney using gene therapy inhibited renal fibrosis and inflammatory pathways. Aspects of the disclosure herein provide methods and compositions that are useful for upregulating SMAD7 for the treatment and/or prevention of diseases associated with reduced SMAD7 expression or function such as acute kidney injury.

### Thalassemia—HAMP

Thalassemia is a group of inherited autosomal recessive blood disorders, resulting in a reduced rate of synthesis or no synthesis of one of the globin chains that make up hemoglobin. This can cause the formation of abnormal hemoglobin molecules or reduced numbers of hemoglobin, thus causing anemia, the characteristic presenting symptom of the thalassemias. HAMP (hepcidin antimicrobial peptide) encodes the protein hepcidin, which plays a major role in maintaining iron balance in the body. Hepcidin circulates in the blood and inhibits iron absorption by the small intestine when the body's supply of iron is too high. HAMP expression has been shown to be lower in patients with thalassemia and is associated with iron-overload (sometimes called hemochromatosis) in these patients. Aspects of the disclosure herein provide methods and compositions that are useful for upregulating HAMP for the treatment and/or prevention of diseases associated with reduced HAMP expression or function such as thalassemia.

### Lesch-Nyhan Disease—HPRT1

Lesch-Nyhan syndrome (LNS), also known as Nyhan's syndrome, Kelley-Seegmiller syndrome and Juvenile gout, is a rare inherited disorder caused by a deficiency of the enzyme hypoxanthine-guanine phosphoribosyltransferase (HGPRT), produced by mutations in the HPRT gene located on the X chromosome. LNS affects about one in 380,000 live births. The HGPRT deficiency causes a build-up of uric acid in all body fluids. This results in both hyperuricemia and hyperuricosuria, associated with severe gout and kidney problems. Neurological signs include poor muscle control and moderate mental retardation. Aspects of the disclosure herein provide methods and compositions that are useful for upregulating HPRT for the treatment and/or prevention of diseases associated with reduced HPRT expression or function such as Lesch-Nyhan syndrome.

### Delayed Growth—IGF-1

Delayed growth is poor or abnormally slow height or weight gains in a child typically younger than age 5. IGF-1, Insulin growth factor-1, is a hormone similar in molecular structure to insulin. IGF-1 plays an important role in childhood growth and continues to have anabolic effects in adults. IGF1 deficiency has been shown to be associated with delayed growth and short stature in humans. Aspects of the disclosure herein provide methods and compositions that are useful for upregulating IGF1 for the treatment and/or prevention of diseases associated with reduced IGF1 expression or function such as delayed growth.

### Dyslipidemias and Atherosclerosis—LDLR

Accumulation of lipids in the blood can cause a variety of conditions and diseases, e.g. dyslipidemia and atherosclerosis. Atherosclerosis in particular is the leading cause of death in industrialized societies, making prevention and treatment a high public health concern. Low-density lipoprotein (LDL) is a major transporter of fat molecules, e.g., cholesterol, in the blood stream that delivers fat molecules to cells. High-density lipoprotein (HDL) is another transporter of fat molecules that moves lipids, e.g., cholesterol, from cells to the liver. High levels of LDL are associated with health problems such as dyslipidemia and atherosclerosis, while HDL is protective against atherosclerosis and is involved in maintenance of cholesterol homeostasis.

Dyslipidemia generally describes a condition when an abnormal amount of lipids is present in the blood. Hyperlipidemia, which accounts for the majority of dyslipidemias, refers to an abnormally high amount of lipids in the blood. Hyperlipidemia is often associated with hormonal diseases such as diabetes, hypothyroidism, metabolic syndrome, and Cushing syndrome. Examples of common lipids in dyslipidemias include triglycerides, cholesterol and fat. Abnormal amounts lipids or lipoproteins in the blood can lead to atherosclerosis, heart disease, and stroke.

Atherosclerosic diseases, e.g. coronary artery disease (CAD) and myocardial infarction (MI), involve a thickening of artery walls caused by accumulation of fat in the blood, most commonly cholesterol. This thickening is thought to be the result of chronic inflammation of arteriole walls due to accumulation of LDLs in the vessel walls. LDL molecules can become oxidized once inside vessel walls, resulting in cell damage and recruitment of immune cells like macrophages to absorb the oxidized LDL. Once macrophages internalize oxidized LDL, they become saturated with cholesterol and are referred to as foam cells. Smooth muscle cells are then recruited and form a fibrous region. These processes eventually lead to formation of plaques that block arteries and can cause heart attack and stroke. HDL is capable of transporting cholesterol from foam cells to the liver, which aids in inhibition of inflammation and plaque formation.

The LDLR gene encodes the Low-Density Lipoprotein (LDL) Receptor, which is a mosaic protein of about 840 amino acids (after removal of signal peptide) that mediates the endocytosis of cholesterol-rich LDL. It is a cell-surface receptor that recognizes the apoprotein B 100 which is embedded in the phospholipid outer layer of LDL particles. LDL receptor complexes are present in clathrin-coated pits (or buds) on the cell surface, which when bound to LDL-cholesterol via adaptin, are pinched off to form clathrin-coated vesicles inside the cell. This allows LDL-cholesterol to be bound and internalized in a process known as endocytosis. This occurs in all nucleated cells (not erythrocytes), but mainly in the liver which removes about 70% of LDL from the circulation. Aspects of the disclosure herein provide methods and compositions that are useful for upregulating LDLR for the treatment and/or prevention of diseases associated with reduced LDLR expression or function such as dyslipidemia or atherosclerosis. Rare, compound heterozygous variants in SCARB1 segregate with severe, premature Cornary artery disease. The maternal variant (c.754_755delinsC) was recently discovered to be the first identified SCARB1 null allele, characterized by the absence of RNA and protein expression. This variant on the paternal allele (c.956G>T [p.G319V]) results in decreased cholesterol uptake, decreased SR-BI:HDL binding, and increased affinity for SR-BI dimerization. p.G319V knock-in mouse model display nearly 100% homozygous lethality and elevated plasma cholesterol in heterozygous animals, further confirming pathogenicity of this variant (Ref: Koenig SN, et al. Circulation Research. 2021;129:296-307).

ATP-binding cassette transporter ABCA1 is a protein which in humans is encoded by the ABCA1 gene. This transporter is a major regulator of cellular cholesterol and phospholipid homeostasis. Mutations in this gene have been associated with Tangier disease and familial high-density lipoprotein deficiency. ABCA1 has been shown to be reduced in Tangier disease which features physiological deficiencies of HDL

### Tissue Regeneration—NANOG

Regeneration is the process of renewal, restoration, and growth of cells and organs in response to disturbance or damage. Strategies for regeneration of tissue include the rearrangement of pre-existing tissue, the use of adult somatic stem cells and the dedifferentiation and/or transdifferentiation of cells, and more than one mode can operate in different tissues of the same animal. During the developmental process, genes are activated that serve to modify the properties of cells as they differentiate into different tissues. Development and regeneration involves the coordination and organization of populations cells into a blastema, which is a mound of stem cells from which regeneration begins, Dedifferentiation of cells means that they lose their tissue-specific characteristics as tissues remodel during the regeneration process. Transdifferentiation of cells occurs when they lose their tissue-specific characteristics during the regeneration process, and then re-differentiate to a different kind of cell. These strategies result in the re-establishment of appropriate tissue polarity, structure and form. NANOG is a transcription factor involved with self-renewal of undifferentiated embryonic stem cells through maintenance of pluripotency. Aspects of the disclosure herein provide methods and compositions that are useful for upregulating NANOG for tissue regeneration.

### Oxidative Stress/Antioxidative Pathway—SIRT6

Cells are protected against oxidative stress by an interacting network of antioxidant enzymes, Oxidation reactions can produce superoxides or free radicals. In turn, these radicals can start chain reactions. When the chain reaction occurs in a cell, it can cause damage or death to the cell. Antioxidants terminate these chain reactions by removing free radical intermediates, and inhibit other oxidation reactions. The superoxide released by processes such as oxidative phosphorylation is first converted to hydrogen peroxide and then further reduced to give water. This detoxification pathway is the result of multiple enzymes, with superoxide dismutases catalysing the first step and then catalases and various peroxidases removing hydrogen peroxide. As oxidative stress appears to be an important part of many human diseases, the use of antioxidants in pharmacology is highly attractive. Mono-ADP-ribosyltransferase sirtuin-6 is an enzyme that in humans is encoded by the SIRT6 gene. Sirtuin-6 has been shown to have a protective role against metabolic damage caused by a high fat diet. SIRT6 deficiency is associated with metabolic defects that lead to oxidative stress. Aspects of the disclosure herein provide methods and compositions that are useful for upregulating SIRT6 for tissue regeneration. Aspects of the disclosure herein provide methods and compositions that are useful for upregulating SIRT6 for the treatment and/or prevention of diseases associated with reduced SIRT6 expression or function such as oxidative stress.

### Choroidal Neovascularization—SERPINF1

Choroidal neovascularization (CNV) is the creation of new blood vessels in the choroid layer of the eye. This is a common symptom of the degenerative maculopathy wet AMD (age-related macular degeneration). Serpin F1 (SERPINF1), also known as Pigment epithelium-derived factor (PEDF), is a multifunctional secreted protein that has anti-angiogenic, anti-tumorigenic, and neurotrophic functions. The anti-angiogenic properties of SERPINF1 allow it to block new blood vessel formation. Aspects of the disclosure herein provide methods and compositions that are useful for upregulating SERPINF1 for the treatment and/or prevention of diseases associated with reduced SERPINF1 expression or function such as Choroidal neovascularization.

### Cardiovascular Disease—SERPINF1

Cardiovascular disease is a class of diseases that involve the heart or blood vessels (arteries and veins). Cardiovascular diseases remain the biggest cause of deaths worldwide. Types of cardiovascular disease include, Coronary heart disease, Cardiomyopathy, Hypertensive heart disease, Heart failure, Corpulmonale, Cardiac dysrhythmias, Inflammatory heart disease, Valvular heart disease, Stroke and Peripheral arterial disease. Serpin F1 (SERPINF1), also known as Pigment epithelium-derived factor (PEDF), is a multifunctional secreted protein that has anti-angiogenic, anti-tumorigenic, and neurotrophic functions, SERPINF1 has been shown to have a protective role in atherosclerosis, the main cause of coronary heart disease, myocardial infarction and heart failure due to its anti-inflammatory, antioxidant and antithrombotic effects in the vessel wall and platelets. Additionally, SERPINF1 has strong antiangiogenic effects by inducing apoptosis in endothelial cells and by regulating the expression of other angiogenic factors.

Aspects of the disclosure herein provide methods and compositions that are useful for upregulating SERPINF1 for the treatment and/or prevention of diseases associated with reduced SERPINF1 expression or function such as cardiovascular disease.

### Hyperimmunoglobulin E Syndrome—STAT3

Loss-of-function mutations in the STAT3 gene result in Hyperimmunoglobulin E syndrome, associated with recurrent infections as well as disordered bone and tooth development.

### Leber's Congenital Amaurosis (LCA), Bardet-Biedl Syndrome (BBS). Joubert Syndrome. Meckel Syndrome, Sior-Loken Syndrome—CEP290

Leber's congenital amaurosis (LCA) is a rare autosomal recessive eye disease resulting in a severe form of retinal dystrophy that is present from birth. LCA results in slow or non-existent pupillary responses, involuntary eye movement, and severe loss of vision. LCA is thought to be caused by abnormal photoreceptor cell development or degeneration. Bardet-Biedl syndrome (BBS) is characterized by retinal dystrophy and retinitis pigmentosa. Other manifestations include polydactyly and renal abnormalities. Both LCA and BBS are associated with mutations in Centrosomal protein 290 kDA (CEP290).

CEP290 is a large coiled-coil protein found in the centrosome and cilia of cells. CEP290 modulates ciliary formation and is involved in trafficking ciliary proteins between the cell body and the cilium of a cell. Reduction or abolishment of CEP290 activity, results in retinal and photoreceptor degeneration. This generation is thought to be the result of defects in ciliogenesis. CEP290 is also associated with Joubert syndrome, Meckel syndrome, and Sior-Loken syndrome. Aspects of the disclosure herein provide methods and compositions that are useful for upregulating CEP290 for the treatment and/or prevention of diseases associated with reduced CEP290 expression or function such as Leber's congenital amaurosis (LCA), Bardet-Biedl syndrome (BBS), Joubert syndrome, Meckel syndrome, Sior-Loken syndrome.

### Phenylketonuria—PAH

Phenylketonuria (PKU) is an autosomal recessive metabolic disease caused by elevated levels of Phenyalanine (Phe) in the blood. Phe is a large neutral amino acid (LNAA) that interacts with the LNAA transporter in order to cross the blood-brain barrier. When Phe is in excess in the blood, it saturates the LNAA transporter, prevent other essential LNAAs from crossing the blood-brain barrier. This results in depletion of these amino acids in the brain, leading to slowing of the development of the brain and mental retardation. PKU can be managed by strictly controlling and monitoring Phe levels in the diet in infants and children. However, if left untreated, severe mental retardation, irregular motor functions, and behavioral disorders result from Phe accumulation in the blood.

Phe accumulation in the blood is the result of mutations in the Phenylalanine hydroxylase (PAH) gene, which encodes phenylalanine hydroxylase protein. Phenylalanine hydroxylase is an enzyme that generates tyrosine through hydroxylation of the aromatic side-chain of Phe. Phenylalanine hydroxylase is the rate-limiting enzyme in the degradation of excess Phe. When phenylalanine hydroxylase levels are decreased or enzyme functionality is compromised, Phe begins to accumulate in the blood, resulting in PKU. Aspects of the disclosure herein provide methods and compositions that are useful for upregulating PAH for the treatment and/or prevention of diseases associated with reduced PAH expression or function such as PKU.

### Congenital Bilateral Absence of Vas Deferens (CBAVD) and Cystic Fibrosis (CF)—CFTR

CFTR is a cyclic-AMP activated ATP-gated anion channel that transports ions across cell membranes. CFTR is predominantly found in epithelial cells in the lung, liver, pancreas, digestive tract, reproductive tract, and skin. A main function of CFTR is to move chloride and thiocyanate ions out of epithelial cells. In order to maintain electrical balance, sodium ions move with the chloride and thiocyanate ions, resulting in an increase of electrolytes outside of the cell. This increase results in movement of water out of the cell by osmosis, creating bodily fluids such as mucus, sweat, and digestive juices, depending on the organ. When CFTR activity is reduced or abolished, ion transport is affected, resulting in reduced water movement out of cells and abnormally viscous bodily fluids (e.g., sticky and viscous mucus, sweat, or digestives juices).

Mutations in CFTR are associated with congenital bilateral absence of vas deferens (CBAVD) and cystic fibrosis. Males with congenital bilateral absence of the vas deferens often have mutations that result in reduced CFTR activity. As a result of these mutations, the movement of water and salt into and out of cells is disrupted. This disturbance leads to the production of a large amount of thick mucus that blocks the developing vas deferens (a tube that carries sperm from the testes) and causes it to degenerate, resulting in infertility.

Cystic fibrosis (CF) is an autosomal recessive disease characterized by overly viscous secretions in the lungs, pancreas, liver, and intestine. In the lungs, difficulty breathing, and frequent infection are common results of mucus build-up. Viscous secretions in the pancreas lead to scarring, fibrosis, and cyst formation which can subsequently lead to diabetes. Additionally, absorption of nutrients in the intestine is decreased due to a lack of digestive enzymes provided by the pancreas. Blockage of the intestine is also common due to thickening of the feces. Aspects of the disclosure herein provide methods and compositions that are useful for upregulating CFTR for the treatment and/or prevention of diseases associated with reduced CFTR expression or function such CBAVD or CF.

### EXEMPLARY EMBODIMENTS

### Exemplary miRNA Sequences

miRNA148 Binding Sites

miR148a DNA has the following sequence:gaggcaaagt totgagacac tocgactctg agtatgatag aagtcagtgc actacagaac titgtetc (SEQ ID NO:32) and miRNA148a RNA has the following sequence GAGGCAAAGUUCUGAGACACUCCGACUCUGAGUAUGAUAGAAGUCAGUGCACUACAGAACUUU GUCUC (SEQ ID NO:33), e.g., a miRNA148 binding site has the following sequence: AGUGCACUG (SEQ ID NO:16), GUGCACUG (SEQ ID NO:17), or GUGCACUG (SEQ ID NO:18),

In one embodiment, a miRNA148 binding site has X1X2X3X4X5X6X7X8X9X10 (SEQ ID NO:25), wherein X1, X2, X3 and X10 are independently absent.

In one embodiment, X1 or X6 independently is not A. In one embodiment, X2, X4 or X9 independently is not G. In one embodiment, X5 or X7 independently is not C. In one embodiment, X3 or X8 independently is not U. In one embodiment at least two nucleotides of X1-X10 differ from SEQ ID NO:16, 17 or 18. In one embodiment at least three nucleotides of X1-X10 differ from SEQ ID NO:16, 17 or 18. In one embodiment at least four nucleotides of X1-X10 differ from SEQ ID NO:16, 17 or 18. In one embodiment, the 5′UTR has one of SEQ ID NO: 16, 17 or 18 and a sequence with one, two or three nucleotide substitutions relative to one of SEQ ID NO: 16, 17 or 18. Those substitutions may be adjacent to each other or interspersed within the sequence.

In one embodiment, miRNA binding sites useful in the methods include but are not limited to 5′-X1X2UGCACUGX10-3′ (SEQ ID NO:26), wherein X1, X2 and X10 are independently absent or are from 1 to 20 ribonucleotides in length, e.g., X1, X2 or X3 are 1, 2, 3, 4, 5, 6, 7, 8, 9, 10, or 15 ribonucleotides in length, as well as the corresponding miRNA DNA sequences, and sequences having at least 90%, 92%, 95%, 96%, or 99% identity thereto. In one embodiment, the ribonucleotides or deoxyribonucleotides include one or more modified ribonucleotides or deoxyribonucleotides, e.g., modified phosphate linkages, modified sugars, modified nucleobases, or combinations thereof. In one embodiment, X1 is A. In one embodiment, X2 is G. In one embodiment, X10 is A.

In one embodiment, miRNA binding sites useful in the methods include but are not limited to 5′-X1X2X3GCACUGX10-3′ (SEQ ID NO:27), wherein X1, X2 and X10 are independently absent or are from 1 to 20 ribonucleotides in length, e.g., X1, X2 or X3 are 1, 2, 3, 4, 5, 6, 7, 8, 9, 10, or 15 ribonucleotides in length, as well as the corresponding miRNA DNA sequences, and sequences having at least 90%, 92%, 95%, 96%, or 99% identity thereto. In one embodiment, the ribonucleotides or deoxyribonucleotides include one or more modified ribonucleotides or deoxyribonucleotides, e.g., modified phosphate linkages, modified sugars, modified nucleobases, or combinations thereof. In one embodiment, X1 is A. In one embodiment, X2 is G. In one embodiment, X3 is U. In one embodiment, X3 is A, C or G. In one embodiment, X10 is A.

In one embodiment, miRNA binding sites useful in the methods include but are not limited to 5′-X1X2X3X4CACUGX10-3′ (SEQ ID NO:28), wherein X1, X2 and X10 are independently absent or are from 1 to 20 ribonucleotides in length, e.g., X1, X2 or X3 are 1, 2, 3, 4, 5, 6, 7, 8, 9, 10, or 15 ribonucleotides in length, as well as the corresponding miRNA DNA sequences, and sequences having at least 90%, 92%, 95%, 96%, or 99% identity thereto. In one embodiment, the ribonucleotides or deoxyribonucleotides include one or more modified ribonucleotides or deoxyribonucleotides, e.g., modified phosphate linkages, modified sugars, modified nucleobases, or combinations thereof. In one embodiment, X1 is A. In one embodiment, X2 is G. In one embodiment, X3 is U. In one embodiment, X4 is G. In one embodiment, X4 is C, A or U. In one embodiment, X10 is A.

In one embodiment, miRNA binding sites useful in the methods include but are not limited to 5′-X1X2X3X4X5ACUGX10-3′ (SEQ ID NO:29), wherein X1, X2 and X10 are independently absent or are from 1 to 20 ribonucleotides in length, e.g., X1, X2 or X3 are 1, 2, 3, 4, 5, 6, 7, 8, 9, 10, or 15 ribonucleotides in length, as well as the corresponding miRNA DNA sequences, and sequences having at least 90%, 92%, 95%, 96%, or 99% identity thereto. In one embodiment, the ribonucleotides or deoxyribonucleotides include one or more modified ribonucleotides or deoxyribonucleotides, e.g., modified phosphate linkages, modified sugars, modified nucleobases, or combinations thereof. In one embodiment, X1 is A. In one embodiment, X2 is G. In one embodiment, X3 is U. In one embodiment, X4 is G. In one embodiment X5 is C. In one embodiment, X5 is G, A or U. In one embodiment, X10 is A.

In one embodiment, miRNA binding sites useful in the methods include but are not limited to 5′-X1X2X3X4X5X6CUGX10-3′ (SEQ ID NO:30), wherein X1, X2 and X10 are independently absent or are from 1 to 20 ribonucleotides in length, e.g., X1, X2 or X3 are 1, 2, 3, 4, 5, 6, 7, 8, 9, 10, or 15 ribonucleotides in length, as well as the corresponding miRNA DNA sequences, and sequences having at least 90%, 92%, 95%, 96%, or 99% identity thereto. In one embodiment, the ribonucleotides or deoxyribonucleotides include one or more modified ribonucleotides or deoxyribonucleotides, e.g., modified phosphate linkages, modified sugars, modified nucleobases, or combinations thereof. In one embodiment, X1 is A. In one embodiment, X2 is G. In one embodiment, X3 is U. In one embodiment, X4 is G. In one embodiment X5 is C. In one embodiment X6 is A. In one embodiment, X6 is U, C or G. In one embodiment, X10 is A.

Exemplary sequences in 5′UTR sequences having a miR148 binding site include but are not limited to:

C-2
(SEQ ID NO: 1)
AGGGAAAUAAGAGAGAAAAGAAGAGUAAGAAGAAAUAUAAGACCCCGGCGCCGCCACCAUG
C-2/1
(SEQ ID NO: 2)
GGGAAAUAAGAGAGAAAAGAAGAGUAAGAAGAAAUAUAAGACCCCGGCGCCGCCACCAUG
C-2/2
(SEQ ID NO: 3)
GGGAAAUAAGAGAGAAAAGAAGAGUAAGAAGAAAUAUAAGACCCCGGCGCCGCCACCAUG
C-3
(SEQ ID NO: 4)
GGGAAAUAAGAGAGAAAAGAAGAGUAAGAAGAAAUAUAAGACCCCGGCGCCGCCACCAUG
C-4
(SEQ ID NO: 5)
GGGAACAAAUGCACUGAAACAAAUGCACUGAAACAAAUGCACUGAGGGAAAUAAGAGAGAAAA
GAAGAGUAAGAAGAAAUAUAAGACCCCGGCGCCGCCACCAUG
5
(SEQ ID NO: 6)
GGGAACAAUUGCACUGAAACAAAUGCACUGAAACAAAUGCACUGAAACAAAUGCACUGAGGGA
AAUAAGUGCACUGAAAAUAAGUGCACUGAAAAUAAGUGCACUGACCCCGGCGCCGCCACCAUG
C-5
(SEQ ID NO: 7)
AGGAACAAUCUGCACUGAAACAAACUGCACUGAAACAAACUGCACUGAAACAAACUGCACUGAG
GGAAAUAAGUGCACUGAAAAUAAGUGCACUGAAAAUAAGUGCACUGACCCCGGCGCCGCCACC
AUG
C-7
(SEQ ID NO: 8)
AGGAAAAGUGCACUGAAUUUAGGGAAAUAAGAGAGAAAAGAAGAGUAAGAAGAAAUAUAAGAC
CCCGGCGCCGCCACCAUG
C-2/7-1
(SEQ ID NO: 9)
AGGAAAAGAGUCCACUGAAUUUAGGGAAAUAAGAGAGAAAAGAAGAGUAAGAAGAAAUAUAAG
ACCCCGGCGCCGCCACCAUG
C-2/7-2
(SEQ ID NO: 10)
AGGAAAAGAGUGCACUGAAUUUAAAAAGAGUGCACUGAGGGAAAUAAGAGAGAAAAGAAGAGU
AAGAAGAAAUAUAAGACCCCGGCGCCGCCACCAUG
C-2/7-2F
(SEQ ID NO: 11)
AGGAAAAGAGUGCACUGAAUUUAAAAAGAGUGCACUGAAUUUAGGGAAAUAAGAGAGAAAAGA
AGAGUAAGAAGAAAUAUAAGACCCCGGCGCCGCCACCAUG
Cx2/7-3
(SEQ ID NO: 12)
AGGAAAAGAGUCCACUGAAUUUAAAAAGAGUGCACUGAAUUUAAAAAGAGUGCACUGAGGGAA
AUAAGAGAGAAAAGAAGAGUAAGAAGAAAUAUAAGACCCCGGCGCCGCCACCAUG
C-8
(SEQ ID NO: 13)
AGGAAUAAACUGCACUGAAAUAAACUGCACUGAAAUAAACUGCACUGAAAUAAAGGGAAAUAAG
AGAGAAAAGAAGAGUAAGAAGAAAUAUAAGACCCCGGCGCCGCCACCAUG
C-9
(SEQ ID NO: 14)
AGGAAUAAACUGCACUGAAAUAAAGGGAAAUAAGAGAGAAAAGAAGAGUAAGAAGAAAUAUAA
GACCCCGGCGCCGCCACCAUG

(SEQ ID NO: 15)
GGGAAAUAAGAGAGAAAAGAAGAGUAAGAAGAAAUAUAAGACCCCGGCG
CCGCCACCAUG

miRNA192-5p Binding Sites

In one embodiment, a miR192-5p binding site has the following sequence CAUAGGUCAG (SEQ ID NO:60).

In one embodiment, a miR192-5p binding site has Y1Y2Y3Y4Y5Y6Y7Y8Y9Y10 (SEQ ID NO:61) wherein Y1, Y2, Y3 and Y10 are independently absent. In one embodiment, Y2, X4 or X9 independently is not A. In one embodiment, Y5, Y6 or Y10 independently is not G. In one embodiment, Y1, or Y8 independently is not C. In one embodiment, Y3, or Y7 independently is not U.

In one embodiment at least two nucleotides of Y1-Y10 differ from SEQ ID NO:60.

In one embodiment at least three nucleotides of Y1-Y10 differ from SEQ ID NO:60.

In one embodiment at least four nucleotides of Y1-Y10 differ from SEQ ID NO:60.

In one embodiment, the 5′UTR has SEQ ID NO:60 and a sequence with one, two or three nucleotide substitutions relative to SEQ ID NO:60. Those substitutions may be adjacent to each other or interspersed within the sequence.

In one embodiment, miRNA binding sites useful in the methods include but are not limited to 5′-Y1Y2UAGGUCAY10-3′ (SEQ ID NO:62), wherein Y1, Y2 and Y10 are independently absent or are from 1 to 20 ribonucleotides in length, e.g., Y1, Y2 or Y3 are 1, 2, 3, 4, 5, 6, 7, 8, 9, 10, or 15 ribonucleotides in length, as well as the corresponding miRNA DNA sequences, and sequences having at least 90%, 92%, 95%, 96%, or 99% identity thereto, In one embodiment, the ribonucleotides or deoxyribonucleotides include one or more modified ribonucleotides or deoxyribonucleotides, e.g., modified phosphate linkages, modified sugars, modified nucleobases, or combinations thereof. In one embodiment, Y1 is C. In one embodiment, Y2 is A. In one embodiment, Y10 is G.

In one embodiment, miRNA binding sites useful in the methods include but are not limited to 5′-Y1Y2Y3AGGUCAY10-3′ (SEQ ID NO:63), wherein Y1, Y2 and Y10 are independently absent or are from 1 to 20 ribonucleotides in length, e.g., Y1, Y2 or Y3 are 1, 2, 3, 4, 5, 6, 7, 8, 9, 10, or 15 ribonucleotides in length, as well as the corresponding miRNA DNA sequences, and sequences having at least 90%, 92%, 95%, 96%, or 99% identity thereto. In one embodiment, the ribonucleotides or deoxyribonucleotides include one or more modified ribonucleotides or deoxyribonucleotides, e.g., modified phosphate linkages, modified sugars, modified nucleobases, or combinations thereof. In one embodiment, Y1 is C. In one embodiment, Y2 is A. In one embodiment, Y3 is U. In one embodiment, Y10 is G.

In one embodiment, miRNA binding sites useful in the methods include but are not limited to 5′-Y1Y2Y3Y4GGUCAY10-3′ (SEQ ID NO:64), wherein Y1, Y2 and Y10 are independently absent or are from 1 to 20 ribonucleotides in length, e.g., Y1, Y2 or Y3 are 1, 2, 3, 4, 5, 6, 7, 8, 9, 10, or 15 ribonucleotides in length, as well as the corresponding miRNA DNA sequences, and sequences having at least 90%, 92%, 95%, 96%, or 99% identity thereto, In one embodiment, the ribonucleotides or deoxyribonucleotides include one or more modified ribonucleotides or deoxyribonucleotides, e.g., modified phosphate linkages, modified sugars, modified nucleobases, or combinations thereof. In one embodiment, Y1 is C. In one embodiment, Y2 is A. In one embodiment, Y3 is U. In one embodiment, Y4 is A, C or U. In one embodiment, Y10 is A.

In one embodiment, miRNA binding sites useful in the methods include but are not limited to 5′-Y1Y2Y3Y4Y5GUCAY10-3′ (SEQ ID NO:65), wherein Y1, Y2 and Y10 are independently absent or are from 1 to 20 ribonucleotides in length, e.g., Y1, Y2 or Y3 are 1, 2, 3, 4, 5, 6, 7, 8, 9, 10, or 15 ribonucleotides in length, as well as the corresponding miRNA DNA sequences, and sequences having at least 90%, 92%, 95%, 96%, or 99% identity thereto. In one embodiment, the ribonucleotides or deoxyribonucleotides include one or more modified ribonucleotides or deoxyribonucleotides, e.g., modified phosphate linkages, modified sugars, modified nucleobases, or combinations thereof. In one embodiment, Y1 is C. In one embodiment, Y2 is A. In one embodiment, Y3 is U. In one embodiment, Y4 is A, U or G. In one embodiment Y5 is G or C. In one embodiment, Y10 is A.

In one embodiment, miRNA binding sites useful in the methods include but are not limited to 5′-Y1Y2Y3Y4Y5Y6UCAY10-3′ (SEQ ID NO:66), wherein Y1, Y2 and Y10 are independently absent or are from 1 to 20 ribonucleotides in length, e.g., Y1, Y2 or Y3 are 1, 2, 3, 4, 5, 6, 7, 8, 9, 10, or 15 ribonucleotides in length, as well as the corresponding miRNA DNA sequences, and sequences having at least 90%, 92%, 95%, 96%, or 99% identity thereto. In one embodiment, the ribonucleotides or deoxyribonucleotides include one or more modified ribonucleotides or deoxyribonucleotides, e.g., modified phosphate linkages, modified sugars, modified nucleobases, or combinations thereof. In one embodiment, Y1 is C. In one embodiment, Y2 is A. In one embodiment, Y3 is U. In one embodiment, Y4 is A, C or G. In one embodiment Y5 is C. In one embodiment Y6 is A or U. In one embodiment, Y6 is U, C or G. In one embodiment, Y10 is A.

Exemplary sequences in 5′UTR sequences having a miR192-5p binding site include but are not limited to:

(SEQ ID NO: 42)
AGGAACAAACAUAGGUCAGAACAAACAUAGGUCAGAACAAACAUAGGUC
AGGGGAAAUAAGAGAGAAAAGAAGAGUAAGAAGAAAUAUAAGACCCCGG
CGCCUUGCCACCAUG;
(SEQ ID NO: 43)
AGGAACAAACAUAGGUCAGGGGAAAUAAGAGAGAAAAGAAGAGUAAGAA
GAAAUAUAAGACCC GCGCCUGCCACCAUG;
(SEQ ID NO: 45)
AGGAACAAACAUAGGUCAGAACAAACAUAGGUCAGAACAGAUACAAUGC
ACGGGAAAUAAGAGAGAAAAGAAGAGUAAGAAGAAAUAUAAGACCCCGU
CGCCUUGCCACCAUG.

miR33a-5p Binding Sites

A miR33a-5p binding site includes UACAAUGCAC (SEQ ID NO:70).

A miR33a-5p binding site includes Z1Z2Z3Z4Z5Z6Z728Z9Z10 (SEQ ID NO:71) wherein Z1, Z2, Z3 and Z10 are independently absent. In one embodiment, Z2, Z4, Z5 or Z9 independently is not A. In one embodiment, Z7 is not G. In one embodiment, Z3, Z8 or Z10 independently is not C. In one embodiment, Z1 or Z7=6 independently is not U.

In one embodiment at least two nucleotides of Z1-210 differ from SEQ ID NO:70

In one embodiment at least three nucleotides of Z1-Z10 differ from SEQ ID NO:70.

In one embodiment at least four nucleotides of Z1-Z10 differ from SEQ ID NO:70.

In one embodiment, the 5′UTR has SEQ ID NO-70 and a sequence with one, two or three nucleotide substitutions relative to SEQ ID NO:70. Those substitutions may be adjacent to each other or interspersed within the sequence.

Exemplary sequences in 5′UTR sequences having a miR33a-5p binding site include but are not limited to:

(SEQ ID NO: 44)
AGGAACAAACAUAGGUCAGAACAAACAUAGGUCAGAACAGAUACAAUGC
ACGGGAAAUAAGAGAGAAAAGAAGAGUAAGAAGAAAUAUAAGACCCCGU
CGCCUUGCCACCAUG
or
(SEQ ID NO: 104)
AGGAACAAAGUAUAAGCUAAACAAAGUAUAAGCUAAACAGAUACAAUGC
ACGGGAAAUAAGAGAGAAAAGAAGAGUAAGAAGAAAUAUAAGACCCCGU
CGUUGCGCCACCAUG.

miRNA21-5p Binding Sites

A miR21-5p binding site includes GUAUAAGCUA (SEQ ID NO:80).

A miR21-5p binding site includes W1W2W3W4W5W6W7W8W9W10 (SEQ ID NO:81), wherein

W1, W2, W3 and W10 are independently absent. In one embodiment, W3, W5, W6 or W10 independently is not A. In one embodiment, W1 or W7 independently is not G. In one embodiment, W8 independently is not C. In one embodiment, W2, W4 or W9 independently is not U.

In one embodiment at least two nucleotides of W1-W10 differ from SEQ ID NO:80.

In one embodiment at least three nucleotides of W1-W10 differ from SEQ ID NO:80.

In one embodiment at least four nucleotides of W1-W10 differ from SEQ ID NO:80.

In one embodiment, the 5′UTR has and a sequence with one, two or three nucleotide substitutions relative to SEQ ID NO:80. Those substitutions may be adjacent to each other or interspersed within the sequence.

In one embodiment, miRNA binding sites useful in the methods include but are not limited to 5′-W1W2AUAAGCU W10-3′ (SEQ ID NO:82), wherein W1, W2 and W10 are independently absent or are from 1 to 20 ribonucleotides in length, e.g., W1, W2 or W3 are 1, 2, 3, 4, 5, 6, 7, 8, 9, 10, or 15 ribonucleotides in length, as well as the corresponding miRNA DNA sequences, and sequences having at least 90%, 92%, 95%, 96%, or 99% identity thereto. In one embodiment, the ribonucleotides or deoxyribonucleotides include one or more modified ribonucleotides or deoxyribonucleotides, e.g., modified phosphate linkages, modified sugars, modified nucleobases, or combinations thereof. In one embodiment, W1 is G. In one embodiment, W2 is U. In one embodiment, W10 is A.

In one embodiment, miRNA binding sites useful in the methods include but are not limited to 5′-W1W2W3UAAGCUW10-3′ (SEQ ID NO:83), wherein W1, W2 and W10 are independently absent or are from 1 to 20 ribonucleotides in length, e.g., W1, W2 or W3 are 1, 2, 3, 4, 5, 6, 7, 8, 9, 10, or 15 ribonucleotides in length, as well as the corresponding miRNA DNA sequences, and sequences having at least 90%, 92%, 95%, 96%, or 99% identity thereto. In one embodiment, the ribonucleotides or deoxyribonucleotides include one or more modified ribonucleotides or deoxyribonucleotides, e.g., modified phosphate linkages, modified sugars, modified nucleobases, or combinations thereof. In one embodiment, W1 is G. In one embodiment, W2 is U. In one embodiment, W3 is A. In one embodiment, W10 is A.

In one embodiment, miRNA binding sites useful in the methods include but are not limited to 5′-W1W2W3W4AAGCUW10-3′ (SEQ ID NO:84), wherein W1, W2 and W10 are independently absent or are from 1 to 20 ribonucleotides in length, e.g., W1, W2 or W3 are 1, 2, 3, 4, 5, 6, 7, 8, 9, 10, or 15 ribonucleotides in length, as well as the corresponding miRNA DNA sequences, and sequences having at least 90%, 92%, 95%, 96%, or 99% identity thereto. In one embodiment, the ribonucleotides or deoxyribonucleotides include one or more modified ribonucleotides or deoxyribonucleotides, e.g., modified phosphate linkages, modified sugars, modified nucleobases, or combinations thereof. In one embodiment, W1 is G. In one embodiment, W2 is U. In one embodiment, W3 is A. In one embodiment, W4 is A, C or U. In one embodiment, W10 is A.

In one embodiment, miRNA binding sites useful in the methods include but are not limited to 5′-W1W2W3W4W5AGCUW10-3′ (SEQ ID NO:85), wherein W1, W2 and W10 are independently absent or are from 1 to 20 ribonucleotides in length, e.g., W1, W2 or W3 are 1, 2, 3, 4, 5, 6, 7, 8, 9, 10, or 15 ribonucleotides in length, as well as the corresponding miRNA DNA sequences, and sequences having at least 90%, 92%, 95%, 96%, or 99% identity thereto. In one embodiment, the ribonucleotides or deoxyribonucleotides include one or more modified ribonucleotides or deoxyribonucleotides, e.g., modified phosphate linkages, modified sugars, modified nucleobases, or combinations thereof. In one embodiment, W1 is G. In one embodiment, W2 is U. In one embodiment, W3 is A. In one embodiment, W4 is A, U or G. In one embodiment W5 is A or U. In one embodiment, W10 is A.

In one embodiment, miRNA binding sites useful in the methods include but are not limited to 5′-W1W2W3W4W5W6UCAW10-3′ (SEQ ID NO:86), wherein W1, W2 and W10 are independently absent or are from 1 to 20 ribonucleotides in length, e.g., W1, W2 or W3 are 1, 2, 3, 4, 5, 6, 7, 8, 9, 10, or 15 ribonucleotides in length, as well as the corresponding miRNA DNA sequences, and sequences having at least 90%, 92%, 95%, 96%, or 99% identity thereto. In one embodiment, the ribonucleotides or deoxyribonucleotides include one or more modified ribonucleotides or deoxyribonucleotides, e.g., modified phosphate linkages, modified sugars, modified nucleobases, or combinations thereof. In one embodiment, W1 is G. In one embodiment, W2 is U. In one embodiment, W3 is U. In one embodiment, W4 is A, U or G. In one embodiment W5 is A or U. In one embodiment W6 is A or U. In one embodiment, W6 is U, C or G. In one embodiment, W10 is A.

Exemplary sequences in 5′UTR sequences having a miR21-5p binding site include but are not limited to:

(SEQ ID NO: 40)
AGGAACAAAGUAUAAGCUAAACAAAGUAUAAGCUAAACAGAGUAUAAGC
UAGGGAAAUAAGAGAGAAAAGAAGAGUAAGAAGAAAUAUAAGACCCCGG
CUUCGCCACCAU;
(SEQ ID NO: 41)
AGGAACAAAGUAUAAGCUAGGGAAAUAAGAGAGAAAAGAAGAGUAAGAA
GAAAUAUAAGACCCCGGCUUCAGCCACCAUG;
or
(SEQ ID NO: 105)
AGGAACAAAGUAUAAGCUAAACAAAGUAUAAGCUAAACAGAUACAAUGC
ACGGGAAAUAAGAGAGAAAAGAAGAGUAAGAAGAAAUAUAAGACCCCGU
CGUUGCGCCACCAUG

### Exemplary Amino Acid Sequences For Expression

In one embodiment the mRNA or vector encoding the mRNA has an open reading frame for a polypeptide comprising phospholipid-transporting ATPase ABCA1 having NCBI Reference Sequence: NP_005493.2:

(SEQ ID NO: 52)
MACWPQLRLL LWKNLTFRRR QTCQLLLEVA WPLFIFLILI SVRLSYPPYE QHECHFPNKA
MPSAGTLPWV QGIICNANNP CFRYPTPGEA PGVVGNFNKS IVARLFSDAR RLLLYSQKDT
SMKDMRKVLR TLQQIKKSSS NLKLQDFLVD NETFSGFLYH NLSLPKSTVD KMLRADVILH
KVFLQGYQLH LTSLCNGSKS EEMIQLGDQE VSELCGLPRE KLAAAERVLR SNMDILKPIL
RTLNSTSPFP SKELAEATKT LLHSLGTLAQ ELFSMRSWSD MRQEVMFLTN VNSSSSSTQI
YQAVSRIVCG HPEGGGLKIK SLNWYEDNNY KALFGGNGTE EDAETFYDNS TTPYCNDLMK
NLESSPLSRI IWKALKPLLV GKILYTPDTP ATRQVMAEVN KTFQELAVFH DLEGMWEELS
PKIWTFMENS QEMDLVRMLL DSRDNDHFWE QQLDGLDWTA QDIVAFLAKH PEDVQSSNGS
VYTWREAFNE TNQAIRTISR FMECVNLNKL EPIATEVWLI NKSMELLDER KFWAGIVFTG
ITPGSIELPH HVKYKIRMDI DNVERTNKIK DGYWDPGPRA DPFEDMRYVW GGFAYLQDVV
EQAIIRVLTG TEKKTGVYMQ QMPYPCYVDD IFLRVMSRSM PLFMTLAWIY SVAVIIKGIV
YEKEARLKET MRIMGLDNSI LWFSWFISSL IPLLVSAGLL VVILKLGNLL PYSDPSVVFV
FLSVFAVVTI LQCFLISTLF SRANLAAACG GIIYFTLYLP YVLCVAWQDY VGFTLKIFAS
LLSPVAFGFG CEYFALFEEQ GIGVQWDNLF ESPVEEDGFN LTTSVSMMLF DTFLYGVMTW
YIEAVFPGQY GIPRPWYFPC TKSYWFGEES DEKSHPGSNQ KRISEICMEE EPTHLKLGVS
IQNLVKVYRD GMKVAVDGLA LNFYEGQITS FLGHNGAGKT TTMSILTGLF PPTSGTAYIL
GKDIRSEMST IRQNLGVCPQ HNVLFDMLTV EEHIWFYARL KGLSEKHVKA EMEQMALDVG
LPSSKLKSKT SQLSGGMQRK LSVALAFVGG SKVVILDEPT AGVDPYSRRG IWELLLKYRQ
GRTIILSTHH MDEADVLGDR IAIISHGKLC CVGSSLFLKN QLGTGYYLTL VKKDVESSLS
SCRNSSSTVS YLKKEDSVSQ SSSDAGLGSD HESDTLTIDV SAISNLIRKH VSEARLVEDI
GHELTYVLPY EAAKEGAFVE LFHEIDDRLS DLGISSYGIS ETTLEEIFLK VAEESGVDAE
TSDGTLPARR NRRAFGDKQS CLRPFTEDDA ADPNDSDIDP ESRETDLLSG MDGKGSYQVK
GWKLTQQQFV ALLWKRLLIA RRSRKGFFAQ IVLPAVFVCI ALVFSLIVPP FGKYPSLELQ
PWMYNEQYTF VSNDAPEDTG TLELLNALTK DPGFGTRCME GNPIPDTPCQ AGEEEWTTAP
VPQTIMDLFQ NGNWTMQNPS PACQCSSDKI KKMLPVCPPG AGGLPPPQRK QNTADILQDL
TGRNISDYLV KTYVQIIAKS LKNKIWVNEF RYGGFSLGVS NTQALPPSQE VNDAIKQMKK
HLKLAKDSSA DRFLNSLGRF MTGLDTKNNV KVWFNNKGWH AISSFLNVIN NAILRANLQK
GENPSHYGIT AFNHPLNLTK QQLSEVALMT TSVDVLVSIC VIFAMSFVPA SFVVFLIQER
VSKAKHLQFI SGVKPVIYWL SNFVWDMCNY VVPATLVIII FICFQQKSYV SSTNLPVLAL
LLLLYGWSIT PLMYPASFVF KIPSTAYVVL TSVNLFIGIN GSVATFVLEL FTDNKLNNIN
DILKSVFLIF PHFCLGRGLI DMVKNQAMAD ALERFGENRF VSPLSWDLVG RNLFAMAVEG
VVFFLITVLI QYRFFIRPRP VNAKLSPLND EDEDVRRERQ RILDGGGQND ILEIKELTKI
YRRKRKPAVD RICVGIPPGE CFGLLGVNGA GKSSTFKMLT GDTTVTRGDA FLNKNSILSN
IHEVHQNMGY CPQFDAITEL LTGREHVEFF ALLRGVPEKE VGKVGEWAIR KLGLVKYGEK
YAGNYSGGNK RKLSTAMALI GGPPVVFLDE PTTGMDPKAR RFLWNCALSV VKEGRSVVLT
SHSMEECEAL CTRMAIMVNG RFRCLGSVQH LKNRFGDGYT IVVRIAGSNP DLKPVQDFFG
LAFPGSVLKE KHRNMLQYQL PSSLSSLARI FSILSQSKKR LHIEDYSVSQ TTLDQVFVNF
AKDQSDDDHL KDLSLHKNQT VVDVAVLTSF LQDEKVKESY V,

or a polypeptide having at least 80%, 85%, 87%, 90%, 92%, 95%, 97%, 98%, or 99% or more amino acid sequence identity to SEQ ID NO:52 or a portion thereof.

In one embodiment the mRNA or vector encoding the mRNA has an open reading frame for a polypeptide comprising low-density lipoprotein receptor isoform 1 precursor [Homo sapiens] having NCBI Reference Sequence: NP_000518.1:

(SEQ ID NO: 53)
MGPWGWKLRW TVALLLAAAG TAVGDRCERN EFQCQDGKCI SYKWVCDGSA ECQDGSDESQ
ETCLSVTCKS GDFSCGGRVN RCIPQFWRCD GQVDCDNGSD EQGCPPKTCS QDEFRCHDGK
CISRQFVCDS DRDCLDGSDE ASCPVLTCGP ASFQCNSSTC IPQLWACDND PDCEDGSDEW
PQRCRGLYVF QGDSSPCSAF EFHCLSGECI HSSWRCDGGP DCKDKSDEEN CAVATCRPDE
FQCSDGNCIH GSRQCDREYD CKDMSDEVGC VNVTLCEGPN KFKCHSGECI TLDKVCNMAR
DCRDWSDEPI KECGTNECLD NNGGCSHVCN DLKIGYECLC PDGFQLVAQR RCEDIDECQD
PDTCSQLCVN LEGGYKCQCE EGFQLDPHTK ACKAVGSIAY LFFTNRHEVR KMTLDRSEYT
SLIPNLRNVV ALDTEVASNR IYWSDLSQRM ICSTOLDRAH GVSSYDTVIS RDIQAPDGLA
VDWIHSNIYW TDSVLGTVSV ADTKGVKRKT LFRENGSKPR AIVVDPVHGF MYWTDWGTPA
KIKKGGLNGV DIYSLVTENI QWPNGITLDL LSGRLYWVDS KLHSISSIDV NGGNRKTILE
DEKRLAHPFS LAVFEDKVFW TDIINEAIFS ANRLTGSDVN LLAENLLSPE DMVLFHNLTQ
PRGVNWCERT TLSNGGCQYL CLPAPQINPH SPKFTCACPD GMLLARDMRS CLTEAEAAVA
TQETSTVRLK VSSTAVRTQH TTTRPVPDTS RLPGATPGLT TVEIVTMSHQ ALGDVAGRGN
EKKPSSVRAL SIVLPIVLLV FLCLGVFLLW KNWRLKNINS INFDNPVYQK TTEDEVHICH
NQDGYSYPSR QMVSLEDDVA, or a polypeptide encoded by

(SEQ ID NO: 106)

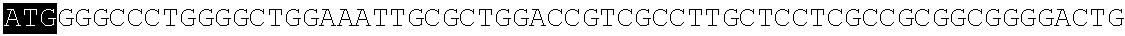

CAGTGGGCGACAGATGCGAAAGAAACGAGTTCCAGTGCCAAGACGGGAAATGCATCTCCTACAAG
TGGGTCTGCGATGGCAGCGCTGAGTGCCAGGATGGCTCTGATGAGTCCCAGGAGACGTGCTTGTC
TGTCACCTGCAAATCCGGGGACTTCAGCTGTGGGGGCCGTGTCAACCGCTGCATTCCTCAGTTCT
GGAGGTGCGATGGCCAAGTGGACTGCGACAACGGCTCAGACGAGCAAGGCTGTCCCCCCAAGACG
TGCTCCCAGGACGAGTTTCGCTGCCACGATGGGAAGTGCATCTCTCGGCAGTTCGTCTGTGACTC
AGACCGGGACTGCTTGGACGGCTCAGACGAGGCCTCCTGCCCGGTGCTCACCTGTGGTCCCGCCA
GCTTCCAGTGCAACAGCTCCACCTGCATCCCCCAGCTGTGGGCCTGCGACAACGACCCCGACTGC
GAAGATGGCTCGGATGAGTGGCCGCAGCGCTGTAGGGGTCTTTACGTGTTCCAAGGGGACAGTAG
CCCCTGCTCGGCCTTCGAGTTCCACTGCCTAAGTGGCGAGTGCATCCACTCCAGCTGGCGCTGTG
ATGGTGGCCCCGACTGCAAGGACAAATCTGACGAGGAAAACTGCGCTGTGGCCACCTGTCGCCCT
GACGAATTCCAGTGCTCTGATGGAAACTGCATCCATGGCAGCCGGCAGTGTGACCGGGAATATGA
CTGCAAGGACATGAGCGATGAAGTTGGCTGCGTTAATGTGACACTCTGCGAGGGACCCAACAAGT
TCAAGTGTCACAGCGGCGAATGCATCACCCTGGACAAAGTCTGCAACATGGCTAGAGACTGCCGG
GACTGGTCAGATGAACCCATCAAAGAGTGCGGGACCAACGAATGCTTGGACAACAACGGCGGCTG
TTCCCACGTCTGCAATGACCTTAAGATCGGCTACGAGTGCCTGTGCCCCGACGGCTTCCAGCTGG
TGGCCCAGCGAAGATGCGAAGATATCGATGAGTGTCAGGATCCCGACACCTGCAGCCAGCTCTGC
GTGAACCTGGAGGGTGGCTACAAGTGCCAGTGTGAGGAAGGCTTCCAGCTGGACCCCCACACGAA
GGCCTGCAAGGCTGTGGGCTCCATCGCCTACCTCTTCTTCACCAACCGGCACGAGGTCAGGAAGA
TGACGCTGGACCGGAGCGAGTACACCAGCCTCATCCCCAACCTGAGGAACGTGGTCGCTCTGGAC
ACGGAGGTGGCCAGCAATAGAATCTACTGGTCTGACCTGTCCCAGAGAATGATCTGCAGCACCCA
GCTTGACAGAGCCCACGGCGTCTCTTCCTATGACACCGTCATCAGCAGAGACATCCAGGCCCCCG
ACGGGCTGGCTGTGGACTGGATCCACAGCAACATCTACTGGACCGACTCTGTCCTGGGCACTGTC
TCTGTTGCGGATACCAAGGGCGTGAAGAGGAAAACGTTATTCAGGGAGAACGGCTCCAAGCCAAG
GGCCATCGTGGTGGATCCTGTTCATGGCTTCATGTACTGGACTGACTGGGGAACTCCCGCCAAGA
TCAAGAAAGGGGGCCTGAATGGTGTGGACATCTACTCGCTGGTGACTGAAAACATTCAGTGGCCC
AATGGCATCACCCTAGATCTCCTCAGTGGCCGCCTCTACTGGGTTGACTCCAAACTTCACTCCAT
CTCAAGCATCGATGTCAACGGGGGCAACCGGAAGACCATCTTGGAGGATGAAAAGAGGCTGGCCC
ACCCCTTCTCCTTGGCCGTCTTTGAGGACAAAGTATTTTGGACAGATATCATCAACGAAGCCATT
TTCAGTGCCAACCGCCTCACAGGTTCCGATGTCAACTTGTTGGCTGAAAACCTACTGTCCCCAGA
GGATATGGTTCTCTTCCACAACCTCACCCAGCCAAGAGGAGTGAACTGGTGTGAGAGGACCACCC
TGAGCAATGGCGGCTGCCAGTATCTGTGCCTCCCTGCCCCGCAGATCAACCCCCACTCGCCCAAG
TTTACCTGCGCCTGCCCGGACGGCATGCTGCTGGCCAGGGACATGAGGAGCTGCCTCACAGAGGC
TGAGGCTGCAGTGGCCACCCAGGAGACATCCACCGTCAGGCTAAAGGTCAGCTCCACAGCCGTAA
GGACACAGCACACAACCACCCGACCTGTTCCCGACACCTCCCGGCTGCCTGGGGCCACCCCTGGG
CTCACCACGGTGGAGATAGTGACAATGTCTCACCAAGCTCTGGGCGACGTTGCTGGCAGAGGAAA
TGAGAAGAAGCCCAGTAGCGTGAGGGCTCTGTCCATTGTCCTCCCCATCGTGCTCCTCGTCTTCC
TTTGCCTGGGGGTCTTCCTTCTATGGAAGAACTGGCGGCTTAAGAACATCAACAGCATCAACTTT
GACAACCCCGTCTATCAGAAGACCACAGAGGATGAGGTCCACATTTGCCACAACCAGGACGGCTA
CAGCTACCCCTCGAGACAGATGGTCAGTCTGGAGGATGACGTGGCG

e.g., one having NCBI Reference Sequence: NM_000527.5, or a polypeptide having at least 80%, 85%, 87%, 90%, 92%, 95%, 97%, 98%, or 99% or more amino acid sequence identity to SEQ ID NO:53 or a portion thereof.

### Exemplary Delivery Vehicles

The nucleic acid described herein may be delivered by any of a variety of vehicles including but not limited to viruses, liposomes, or other nanoparticles. The nucleic acid may form complexes with one or more non-nucleic acid molecules or may be encapsulated in or on the surface of delivery vehicles such as nanoparticles.

Numerous lipids which are used in liposome delivery systems may be used to form a lipid layer, e.g., a bilayer. Exemplary lipids for use include, for example, 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-dioleoyl-sn-glycero-3-[phosphor-L-serine] (DOPS), 1,2-dioleoyl-3-trimethylammonium-propane (18:1 DOTAP), 1,2-dioleoyl-sn-glycero-3-phospho-(1′-rac-glycerol) (DOPG), 1,2-dioleoyl-sn-glycero-3-phosphoethanolamine (DOPE), 1,2-dipalmitoyl-sn-glycero-3-phosphoethanolamine (DPPE), 1,2-dioleoyl-sn-glycero-3-phosphoethanolamine-N-[methoxy(polyethylene glycol)-2000] (18:1 PEG-2000 PE), 1,2-dipalmitoyl-sn-glycero-3-phosphoethanolamine-N-[methoxy(polyethylene glycol)-2000] (16:0 PEG-2000 PE), 1- oleoyl-2-[12-[(7-nitro-2-1,3-benzoxadiazol-4-yl)amino]lauroyl]-sn-glycero-3-phosphocholine (18:1-12:0 NBD PC), 1-palmitoyl-2-{12-[(7-nitro-2-1,3-benzoxadiazol-4-yl)amino]lauroyl}-sn-glycero-3-phosphocholine (16:0-12:0 NBD PC), cholesterol and mixtures/combinations thereof. Cholesterol, not technically a lipid, but presented as a lipid for purposes of an embodiment. Often cholesterol is incorporated into lipid bi-layers to enhance structural integrity of the bi-layer. DOPE and DPPE may be particularly useful for conjugating (through an appropriate crosslinker) a targeting moiety, e.g., a liver targeting moiety on the lipid.

In one embodiment, anionic liposomal nanoparticles are employed as a delivery vehicle for the nucleic acid molecules, wherein the anionic liposomal nanoparticles optionally comprise one or more targeting moieties. In one embodiment, the anionic liposomal nanoparticles have diameters of about 100 nm to about 500 nm. In one embodiment, the anionic liposomal nanoparticles have diameters of about 150 nm to about 250 nm. In one embodiment, the lipid layer comprises lipids including but not limited to 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-dioleoyl-sn-glycero-3-[phosphor-L-serine] (DOPS), 1,2-dioleoyl-3-trimethylammonium-propane (18:1 DOTAP), 1,2-dioleoyl-sn-glycero-3-phospho-(1′-rac-glycerol) (DOPG), 1,2-dioleoyl-sn-glycero-3-phosphoethanolamine (DOPE), 1,2-dipalmitoyl-sn-glycero-3-phosphoethanolamine (DPPE), 1,2-dioleoyl-sn-glycero-3-phosphoethanolamine-N-[methoxy(polyethylene glycol)-2000] (18:1 PEG-2000 PE), 1,2-dipalmitoyl-sn-glycero-3-phosphoethanolamine-N-[methoxy(polyethylene glycol)-2000] (16:0 PEG-2000 PE), 1- oleoyl-2-[12-[(7-nitro-2-1,3-benzoxadiazol-4-yl)amino]lauroyl]-sn-glycero-3-phosphocholine (18:1-12:0 NBD PC), 1-palmitoyl-2-[12-[(7-nitro-2-1,3-benzoxadiazol-4-yl)amino]lauroyl]-sn-glycero-3-phosphocholine (16:0-12:0 NBD PC), and mixtures thereof; or wherein said lipid layer comprises 1,2-distearoyl-sn-glycero-3-phosphocholine (DSPC), 1,2-dioleoyl-sn-glycero-3-phosphoethanolamine (DOPE), or a mixture thereof; or wherein said lipid layer comprises cholesterol. In one embodiment, the lipid layer comprises two or more of DPPC, DMPG or cholesterol.

In certain embodiments, liposomes generally range in size from about 8 to 10 nm to about 5 μm in diameter, e.g., about 20-nm to 3 um in diameter, about 10 nm to about 500 nm, about 20-200-nm (including about 150 nm, which may be a mean or median diameter), about 50 nm to about 150 nm, about 75 to about 130 nm, or about 75 to about 100 nm as well as about 200 to about 450 nm, about 100 to about 200 nm, about 150 to about 250 nm, or about 200 to about 300 nm.

In certain embodiments, the delivery vehicle may be a biodegradable polymer comprising one or more aliphatic polyesters, poly (lactic acid) (PLA), poly (glycolic acid) (PGA), co-polymers of lactic acid and glycolic acid (PLGA), polycaprolactone (PCL), polyanhydrides, poly(ortho)esters, polyurethanes, poly(butyric acid), poly(valeric acid), poly(lactide-co-caprolactone), alginate and other polysaccharides, collagen, and chemical derivatives thereof, albumin a hydrophilic protein, zein, a prolamine, a hydrophobic protein, and copolymers and mixtures thereof.

In other embodiments, the lipid bi-layer is comprised of a mixture of DSPC, DOPC and optionally one or more phosphatidyl-cholines (PCs) selected from the group consisting of 1,2-dimyristoyl-sn-glycero-3-phosphocholine (DMPC), 1,2-dioleoyl-3-trimethylammonium-propane (DOTAP), 1-palmitoyl-2-oleoyl-sn-glycero-3-phosphocholine (POPC), a lipid mixture comprising (in molar percent) between about 50% to about 70% or about 51% to about 69%, or about 52% to about 68%, or about 53% to about 67%, or about 54% to about 66%, or about 55% to about 65%, or about 56% to about 64%, or about 57% to about 63%, or about 58% to about 62%, or about 59% to about 61%, or about 60%, of one or more unsaturated phosphatidyl-choline, DMPC [14:0] having a carbon length of 14 and no unsaturated bonds, 1,2-dipalmitoyl-sn-glycero-3-phosphocholine (DPPC) [16:0], POPC [16:0-18:1] and DOTAP [18:1]; and wherein (b) the molar concentration of DSPC and DOPC in the mixture is between about 10% to about 99% or about 50% to about 99%, or about 12% to about 98%, or about 13% to about 97%, or about 14% to about 96%, or about 55% to about 95%, or about 56% to about 94%, or about 57% to about 93%, or about 58% to about 42%, or about 59% to about 91%, or about 50% to about 90%, or about 51% to about 89%.

In certain embodiments, the lipid bi-layer is comprised of one or more compositions selected from the group consisting of a phospholipid, a phosphatidyl-choline, a phosphatidyl-serine, a phosphatidyl-diethanolamine, a phosphatidylinosite, a sphingolipid, and an ethoxylated sterol, or mixtures thereof. In illustrative examples of such embodiments, the phospholipid can be a lecithin; the phosphatidylinosite can be derived from soy, rape, cotton seed, egg and mixtures thereof; the sphingolipid can be ceramide, a cerebroside, a sphingosine, and a sphingomyelin, and a mixture thereof; the ethoxylated sterol can be phytosterol, PEG-(polyethyleneglycol)-5-soy bean sterol, and PEG-(polyethyleneglycol)-5 rapeseed sterol. In certain embodiments, the phytosterol comprises a mixture of at least two of the following compositions: sitosterol, campesterol and stigmasterol.

In still other illustrative embodiments, the lipid bi-layer is comprised of one or more phosphatidyl groups selected from the group consisting of phosphatidyl choline, phosphatidyl-ethanolamine, phosphatidyl-serine, phosphatidyl- inositol, lyso-phosphatidyl-choline, lyso-phosphatidyl-ethanolamine, lyso-phosphatidyl-inositol and lyso-phosphatidyl-inositol.

In still other illustrative embodiments, the lipid bi-layer is comprised of phospholipid selected from a monoacyl or diacylphosphoglyceride.

In still other illustrative embodiments, the lipid bi-layer is comprised of one or more phosphoinositides selected from the group consisting of phosphatidyl-inositol-3-phosphate (PI-3-P), phosphatidyl-inositol-4-phosphate (PI-4-P), phosphatidyl-inositol-5-phosphate (PI-5-P), phosphatidyl-inositol-3,4-diphosphate (PI-3,4-P2), phosphatidyl-inositol-3,5-diphosphate (PI-3,5-P2), phosphatidyl-inositol-4,5-diphosphate (PI-4,5-P2), phosphatidyl-inositol-3,4,5-triphosphate (PI-3,4,5-P3), lysophosphatidyl-inositol-3-phosphate (LPI-3-P), lysophosphatidyl-inositol-4-phosphate (LPI-4-P), lysophosphatidyl-inositol-5-phosphate (LPI-5-P), lysophosphatidyl-inositol-3,4-diphosphate (LPI-3,4-P2), lysophosphatidyl-inositol-3,5-diphosphate (LPI-3,5-P2), lysophosphatidyl-inositol-4,5-diphosphate (LPI-4,5-P2), and lysophosphatidyl-inositol-3,4,5-triphosphate (LPI-3,4,5-P3), and phosphatidyl-inositol (PI), and lysophosphatidyl-inositol (LPI).

In still other illustrative embodiments, the lipid bi-layer is comprised of one or more phospholipids selected from the group consisting of PEG-poly(ethylene glycol)-derivatized distearoylphosphatidylethanolamine (PEG-DSPE), PEG-poly(ethylene glycol)-derivatized dioleoylphosphatidylethanolamine (PEG-DOPE), poly(ethylene glycol)-derivatized ceramides (PEG-CER), hydrogenated soy phosphatidylcholine (HSPC), egg phosphatidylcholine (EPC), phosphatidyl ethanolamine (PE), phosphatidyl glycerol (PG), phosphatidyl inositol (PI), monosialoganglioside, sphingomyelin (SPM), distearoylphosphatidylcholine (DSPC), dimyristoylphosphatidylcholine (DMPC), and dimyristoylphosphatidylglycerol (DMPG).

In still other embodiments, the lipid bi-layer comprises one or more PEG-containing phospholipids, for example 1,2-dioleoyl-sn-glycero-3-phosphoethanolamine-N-[methoxy(polyethylene glycol)] (ammonium salt) (DOPE-PEG), 1,2-distearoyl-sn-glycero-3-phosphoethanolamine-N-[methoxy(polyethylene glycol)] (ammonium salt) (DSPE-PEG), 1,2-distearoyl-sn-glycero-3- phosphoethanolamine-N-[amino(polyethylene glycol)] (DSPE-PEG-NH2) (DSPE-PEG), In the PEG-containing phospholipid, the PEG group ranges from about 2 to about 250 ethylene glycol units, about 5 to about 100, about 10 to 75, or about 40-50 ethylene glycol units. In certain exemplary embodiments, the PEG-phospholipid is 1,2-dioleoyl-sn-glycero-3-phosphoethanolamine-N-[methoxy(polyethylene glycol)-2000] (ammonium salt) (DOPE-PEG₂₀₀₀), 1,2-distearoyl-sn-glycero-3-phosphoethanolamine-N-[methoxy(polyethylene glycol)-2000] (ammonium salt) (DSPE-PEG₂₀₀₀), 1,2-distearoyl-sn-glycero-3-phosphoethanolamine-N-[amino(polyethylene glycol)-2000] (DSPE-PEG₂₀₀₀-NH2) which can be used to covalent bind a functional moiety to the lipid bi-layer.

In certain embodiments, the lipid bi-layer is comprised of one or more phosphatidylcholines (PCs) selected from the group consisting of 1,2-distearoyl-sn-glycero-3-phosphocholine (DSPC) [18:0], 1,2-dioleoyl-sn-glycero-3-phosphocholine (DOPC) [18:1 (Δ9-Cis)], 1,2-dimyristoyl-sn-glycero-3-phosphocholine (DMPC), 1,2-dioleoyl-3-trimethylammonium-propane (DOTAP), 1-palmitoyl-2-oleoyl-sn-glycero-3-phosphocholine (POPC), egg PC, and a lipid mixture comprising of one or more unsaturated phosphatidyl-cholines, DMPC [14:0] having a carbon length of 14 and no unsaturated bonds, 1,2-dipalmitoyl-sn-glycero-3-phosphocholine (DPPC) [16:0], POPC [16:0-18:1], and DOTAP [18:1]. The use of DSPC and/or DOPC as well as other zwitterionic phospholipids as a principal component (often in combination with a minor amount of cholesterol) is employed in certain embodiments in order to provide a protocell with a surface zeta potential which is neutral or close to neutral in character.

Cationic liposomes may be formed from a single type of lipid, or a combination of two or more distinct lipids. For instance, one combination may include a cationic lipid and a neutral lipid, or a cationic lipid and a non-cationic lipid. Exemplary lipids for use in the cationic liposomes include but are not limited to DOTAP, DODAP, DDAB, DOTMA, MVL5, DPPC, DSPC, DOPE, DPOC, POPC, or any combination thereof. In one embodiment, the cationic liposome has one or more of the following lipids or precursors thereof: N-[1-(2,3-dioleyloxy)propyl]-N,N,N-trimethylammonium chloride with a monovalent cationic head; N,N-dioctadecyl-N-4,8-diaza-10-aminodecanoyl glycine amide; 1,4,7,10-tetraazacyclododecane cyclen; imidazolium-containing cationic lipid having different hydrophobic regions (e.g., cholesterol and diosgenin); 1,2-dioleoyl-sn-glycero-3-phosphoethanolamine (DOPE); 3-β-[N-(N,N′-dimethylamino-ethane) carbamoyl) cholesterol (DC-Chol) and DOPE; O,O′-ditetradecanoyl-N-(α-trimethyl ammonioacetyl) diethanol-amine chloride, DOPE and cholesterol, phosphatidylcholine; 1,2-dilinoleyl-4-(2-dimethylaminoethyl)-[1,3]-dioxolane, 1,2-distearoyl-sn-glycerol-3-phosphocholine (DSPC) and cholesterol, 1-palmitoyl-2-oleoyl-sn-glycero-3-phosphocholine, DOPE, and 1,2-distearoyl-sn-glycero-3-phosphoethanolamine-N- (methoxy[polyethylene glycol-2000), 1,2-di-O-octadecenyl-3-trimethylammonium propane, cholesterol, and D-α-toco; 1,2-dioleoyl-3-trimethylammonium-propane, cholesterol; 3-β(N-(N,N-dimethyl, N-hydroxyethyl amino-propane) carbamoyl) cholesterol iodide, DMHAPC-Chol and DOPE in equimolar proportion, or 1-palmitoyl-2-oleoyl-sn-glycero-3-ethylphosphocholine:cholesterol, dimethyldioctadecylammonium (DDAB); 1,2-di-O-octadecenyl-3-trimethylammonium propane;N1-[2-((1S)-1-{(3-aminopropyl)amino]-4-[di(3-amino-propyl)amino)amino]butylcarboxamido)ethyl]-3,4-di[oleyloxy]-benzamide (MVL5); 1,2-dioleoyl-3-dimethylammonium-propane (DODAP); 1,2-di-O-octadecenyl-3-trimethylammonium propane (DOTMA); 1,2-dioleoyl-sn-glycero-3-phosphocholine (DOPC);1-palmitoyl-2-oleoyl-sn-glycero-3-phosphocholine (POPC).

### Exemplary Routes and Formulations

Administration of compositions having one or more nucleic acid molecules disclosed herein, can be via any of suitable route of administration, particularly parenterally, for example, intravenously, intra-arterially, intraperitoneally, intrathecally, intraventricularly, intracranially, intramuscularly, or subcutaneously. Such administration may be as a single bolus injection, multiple injections, or as a short- or long-duration infusion. Implantable devices (e.g., implantable infusion pumps) may also be employed for the periodic parenteral delivery over time of equivalent or varying dosages of the particular formulation. For such parenteral administration, the nucleic acid compounds may be formulated as a sterile solution in water or another suitable solvent or mixture of solvents. The solution may contain other substances such as salts, sugars (particularly glucose or mannitol), to make the solution isotonic with blood, buffering agents such as acetic, citric, and/or phosphoric acids and their sodium salts, and preservatives.

The compositions alone or in combination with other active agents can be formulated as pharmaceutical compositions and administered to a mammalian host, such as a human patient in a variety of forms adapted to the chosen route of administration, e.g., orally or parenterally, by intravenous, intramuscular, topical or subcutaneous routes.

Thus, the compositions alone or in combination with another active agent, may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the composition having nucleic acid, optionally in combination with another active compound, may be combined with one or more excipients and used in the form of ingestible tablets, buccal tablets, troches, capsules, elixirs, suspensions, syrups, wafers, and the like, Such compositions and preparations should contain at least 0.1% of active compound. The percentage of the compositions and preparations may, of course, be varied and may conveniently be between about 2 to about 60% of the weight of a given unit dosage form. The amount of the nucleic acid and optionally other active compound in such useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the composition optionally in combination with another active compound may be incorporated into sustained-release preparations and devices.

The composition having nucleic acid optionally in combination with another active compound may also be administered intravenously or intraperitoneally by infusion or injection. Solutions of the nucleic acid molecule optionally in combination with another active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the nucleic acid which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of particle size in the case of dispersions or by the use of surfactants. The prevention of the action of microorganisms during storage can be brought about by various antibacterial and antifungal agents, for example, parabens, chlorobutanol, phenol, sorbic acid, thimerosal, and the like. In many cases, it may be useful to include isotonic agents, for example, sugars, buffers or sodium chloride. Prolonged absorption of the injectable compositions can be brought about by the use in the compositions of agents delaying absorption, for example, aluminum monostearate and gelatin, or a combination thereof.

For example, sterile injectable solutions are prepared by incorporating compound(s) in an effective amount in the appropriate solvent with various of the other ingredients enumerated above, followed by filter sterilization. Generally, dispersions can be prepared by incorporating the selected sterilized active ingredient(s), e.g., via filer sterilization, into a sterile vehicle that contains the basic dispersion medium and any other optional ingredients from those enumerated above. The compositions disclosed herein may also be formulated in a neutral or salt form. Upon formulation, solutions will be administered in a manner compatible with the dosage formulation, and in such amount as is effective for the intended application. The formulations are readily administered in a variety of dosage forms such as injectable solutions, topical preparations, oral formulations, including sustain-release capsules, hydrogels, colloids, viscous gels, transdermal reagents, intranasal and inhalation formulations, and the like. For administration of an injectable aqueous solution, without limitation, the solution may be suitably buffered, if necessary, and the liquid diluent first rendered isotonic with sufficient saline or glucose. These particular aqueous solutions are especially suitable for intravenous, intramuscular, subcutaneous, transdermal, subdermal, and/or intraperitoneal administration. In this regard, the compositions of the present disclosure may be formulated in one or more pharmaceutically acceptable vehicles, including for example sterile aqueous media, buffers, diluents, and the like. For example, a given dosage of active ingredient(s) may be dissolved in a particular volume of an isotonic solution (e.g., an isotonic NaCI-based solution), and then injected at the proposed site of administration, or further diluted in a vehicle suitable for intravenous infusion (see, e.g., “REMINGTON'S PHARMACEUTICAL SCIENCES” 15^{th} Ed., pp. 1035-1038 and 1570-1580). While some variation in dosage will necessarily occur depending on the condition of the subject being treated, the extent of the treatment, and the site of administration, the person responsible for administration will nevertheless be able to determine the correct dosing regimens appropriate for the individual subject using ordinary knowledge in the medical and pharmaceutical arts.

In the case of sterile powders for the preparation of sterile injectable solutions, one method of preparation includes vacuum drying and the freeze drying techniques, which yield a powder of the active ingredient plus any additional desired ingredient present in the previously sterile-filtered solutions.

For topical administration, the composition optionally in combination with another active compound may be applied in pure form, e.g., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and antimicrobial agents can be added to optimize the properties for a given use.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

In addition, in one embodiment, the disclosure provides various dosage formulations of the nucleic acid optionally in combination with another active compound for inhalation delivery. For example, formulations may be designed for aerosol use in devices such as metered-dose inhalers, dry powder inhalers and nebulizers.

Useful dosages can be determined by comparing their in vitro activity, and in vivo activity in animal models. Methods for the extrapolation of effective dosages in mice, and other animals, to humans are known to the art; for example, see U.S. Pat. No. 4,938,949.

Generally, the concentration of the nucleic acid optionally in combination with another active compound in a liquid, solid or gel composition, may be from about 0.1-25 wt-%, e.g., from about 0.5-10 wt-%, from 10 to 30 wt-%, 30 to 50 -wt %, 50 to 70-wt %, or about 70 to 90 wt-%. The concentration in a semi-solid or solid composition such as a gel or a powder may be about 0.1-5 wt-%, e.g., about 0.5-2.5 wt-% or about 0.5-10 wt-%, from 10 to 30 wt-%, 30 to 50 -wt %, 50 to 70-wt %, or about 70 to 90 wt-%.

The active ingredient may be administered to achieve peak plasma concentrations of the active compound of from about 0.5 to about 75 μM, e.g., about 1 to 50 μM, such as about 2 to about 30 μM. This may be achieved, for example, by the intravenous injection of a 0.05 to 5% solution of the active ingredient, optionally in saline, or orally administered as a bolus containing about 1-100 mg of the active ingredient. Desirable blood levels may be maintained by continuous infusion to provide about 0.01-5.0 mg/kg/hr or by intermittent infusions containing about 0.4-15 mg/kg of the active ingredient(s).

The amount of the nucleic acid optionally in combination with another active compound, or an active salt or derivative thereof, for use in treatment will vary not only with the particular salt selected but also with the route of administration, the nature of the condition being treated and the age and condition of the patient and will be ultimately at the discretion of the attendant physician or clinician. In general, however, a suitable dose may be in the range of from about 0.5 to about 100 mg/kg, e.g., from about 10 to about 75 mg/kg of body weight per day, such as 3 to about 50 mg per kilogram body weight of the recipient per day, for instance in the range of 6 to 90 mg/kg/day, e.g., in the range of 15 to 60 mg/kg/day. In one embodiment, 1 mg/kg to 100 mg/kg, e.g., per day, is administered. In one embodiment, 1 mg/kg to 20 mg/kg. e.g., per day, is administered. In one embodiment, 20 mg/kg to 40 mg/kg, e.g., per day, is administered. In one embodiment, 40 mg/kg to 60 mg/kg, e.g., per day, is administered. In one embodiment, 60 mg/kg to 80 mg/kg, e.g., per day, is administered. In one embodiment, 80 mg/kg to 100 mg/kg, e.g., per day, is administered. The nucleic acid optionally in combination with another active compound may be conveniently administered in unit dosage form; for example, containing 5 to 1000 mg, conveniently 10 to 750 mg, most conveniently, 50 to 500 mg of active ingredient per unit dosage form.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye. The dose, and perhaps the dose frequency, will also vary according to the age, body weight, condition, and response of the individual patient. In general, the total daily dose range for an active agent for the conditions described herein, may be from about 50 mg to about 5000 mg, in single or divided doses. In one embodiment, a daily dose range should be about 100 mg to about 4000 mg, e.g., about 1000-3000 mg, in single or divided doses, e.g., 750 mg every 6 hr of orally administered compound. This may achieve plasma levels of about 500-750 μM, In managing the patient, the therapy should be initiated at a lower dose and increased depending on the patient's global response.

The amount, dosage regimen, formulation, and administration of nucleic acid disclosed herein will be within the purview of the ordinary-skilled artisan having benefit of the present teaching. It is likely, however, that the administration of a therapeutically-effective amount of the disclosed compositions may be achieved by multiple, or successive administrations, over relatively short or even relatively prolonged periods, as may be determined by the medical practitioner overseeing the administration of such compositions to the selected individual. However, a single administration, such as, without limitation, a single injection of a sufficient quantity of the delivered agent may provide the desired benefit to the patient for a period of time.

In certain embodiments, the present disclosure concerns formulation of one or more cationic nanoparticles, e.g., cationic liposomes, for administration to an animal. In one embodiment, a cationic liposome comprises two or more distinct lipids, one of the lipids is cationic, e.g., DOTAP is a cationic lipid, and at least one of the others is non-cationic, e.g., DPPC or DSPC. Ratios of the two or more distinct lipids can vary, for example, for two distinct lipids, the ratio of a non-cationic lipid, e.g., neutral lipid, to the cationic lipid may be x:1 wherein x>1, x=1 or x:1 where x<1. In one embodiment, x>1. The formulation of pharmaceutically acceptable excipients and carrier solutions is well known to those of ordinary skill in the art, as is the development of suitable dosing and treatment regimens for using the particular cationic nanoparticle compositions described herein in a variety of treatment regimens. In certain circumstances it will be desirable to deliver the disclosed compositions in suitably-formulated pharmaceutical vehicles by one or more standard delivery methods, including, without limitation, subcutaneously, parenterally, intravenously, intramuscularly, intrathecally, orally, intraperitoneally, transdermally, topically, by oral or nasal inhalation, or by direct injection to one or more cells, tissues, or organs within or about the body of an animal. The methods of administration may also include those modalities as described in U.S. Pat. Nos. 5,543,158; 5,641,515, and 5,399,363, each of which is specifically incorporated herein in its entirety by express reference thereto. Solutions of the active compounds as freebase or pharmacologically acceptable salts may be prepared in sterile water, and may be suitably mixed with one or more surfactants, such as hydroxypropylcellulose. Dispersions may also be prepared in glycerol, liquid polyethylene glycols, oils, or mixtures thereof. Under ordinary conditions of storage and use, these preparations contain a preservative to prevent the growth of microorganisms.

### Exemplary Methods

In one embodiment, a method to prevent, inhibit or treat liver or cardiovascular disease in a mammal is provided. In one embodiment, a method to inhibit or treat liver disease in a mammal is provided. In one embodiment, the disease is coronary heart disease. In one embodiment, the disease is stroke. In one embodiment, the disease is peripheral vascular disease. In one embodiment, the disease is atherosclerosis In one embodiment, a method to inhibit or treat atherosclerosis in a mammal is provided. In one embodiment, the method includes administering to a mammal in need thereof an effective amount of a composition comprising a nucleic acid sequence comprising a miRNA binding site for a miRNA expressed in the liver, e.g., miRNA148, miR192 or miRNA-33a-5p. In one embodiment, the method includes administering to a mammal in need thereof an effective amount of a composition comprising a nucleic acid sequence comprising a miRNA binding site for a miRNA expressed in macrophage, e.g., miRNA21 or miRNA-33a-5p. In one embodiment, the mammal is a human. In one embodiment, the disease is hypercholesteremia, steatosis, non-alcoholic fatty liver disease (NAFLD), or nonalcoholic steatohepatitis (NASH). In one embodiment the mammal has high cholesterol levels. In one embodiment, the mammal has alcohol fatty liver disease or chronic liver disease. In one embodiment, the mammal has atherosclerosis or complications thereof. In one embodiment, the mammal has hyperlipidemia or complications thereof. In one embodiment, the mammal has dyslipidemia or complications thereof. In one embodiment, the mammal has hypercholesteremia or complications thereof. In one embodiment, the composition comprises liposomes, e.g., cationic liposomes. In one embodiment, the liposomes comprise or more of DC-cholesterol, 1,2-dioleoyl-sn-glycero-3-phosphoethanolamine (DOPE), an ionizable cationic lipid or a lipidoid. In one embodiment, the composition comprises nanoparticles. In one embodiment, the composition is systemically administered. In one embodiment, the composition is orally administered. In one embodiment, the composition is injected. In one embodiment, the nucleic acid sequence is less than 30 bases in length. In one embodiment, the nucleic acid sequence is less than 25 bases in length. In one embodiment, the miRNA binding site is less than 20 bases in length. In one embodiment, the miRNA binding site is 7 to 10 bases in length. In one embodiment, the composition comprises RNA comprising a hairpin-loop structure. In one embodiment, the nucleic acid sequence comprises non-native nucleotides. In one embodiment, the RNA or the one strand comprises non-native nucleotides. In one embodiment, the non-native nucleotide has a modified nucleobase, modified phosphate group or a modified sugar. In one embodiment, the amount of the nucleic acid sequence is about 0.01 mg/kg to about 100 mg/kg. In one embodiment, the amount of the nucleic acid sequence is about 0.05 mg/kg to about 10 mg/kg. In one embodiment, the amount of the nucleic acid sequence is about 10 mg/kg to about 75 mg/kg.

The invention will be described by the following non-limiting examples.

### EXAMPLE 1

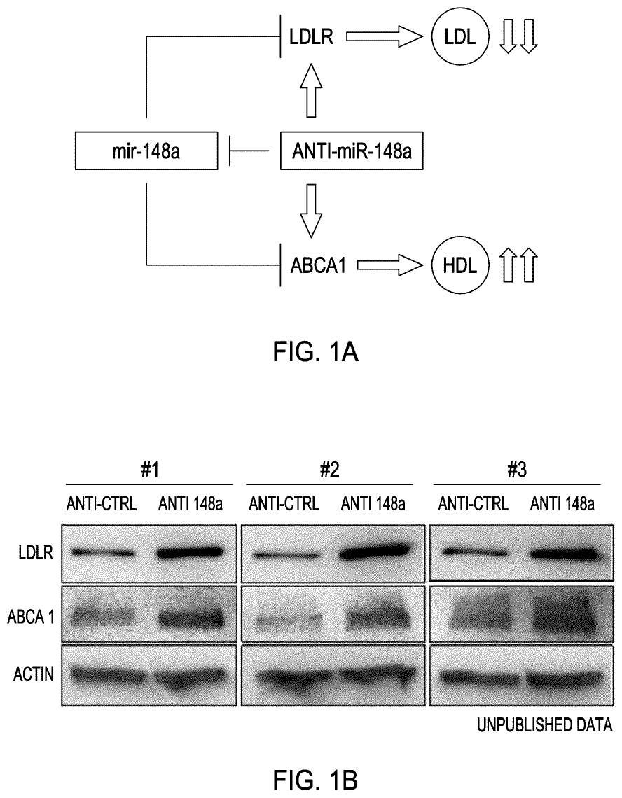

MicroRNAs are regulatory RNAs that inhibit gene expression potentially at early step of translational initiation through direct binding to target mRNAs at 3′-UTR in a sequence specific manner. They are involved in major aspects of human biology as well as disease and some of them have been identified as potential therapeutic targets. Their expression pattern varies among metazoans with some being only expressed in a cell or tissue-specific manner. miR-148, a highly conserved microRNA among mammals with a high-level of liver-specific expression profile, is a regulator of lipoprotein metabolism. Humans carrying MIR148A loss of function mutations have reduced LDL-C levels. LDL receptor (LDLR) and ABCA1 cholesterol transporter involved in LDL-uptake and cholesterol efflux, respectively, as direct targets of miR-148 for post-transcriptional inhibition, Conversely, antisense-mediated inhibition of miR-148a was found to reduce serum LDL-cholesterol levels in mice (FIG. 1).

A combinatorial approach to develop mRNA-based therapeutics was investigated that benefits from both microRNAs and synthetic mRNAs for the treatment of severe dyslipidemia in FH. The strategy allows for generating liver-targeted LDLR mRNAs which translation initiation depends on miR148a-interacting cis-regulatory elements embedded at their 5′UTR. The design is based on an impinged secondary structure that specifically folds into a more linearized conformation upon binding to the liver-enriched miR-148a effector in trans and leads to an allosteric switch in the LDLR mRNA that promotes its translation initiation in a liver-specific manner.

### Structure Design

The 5′-untranslated region (5′-UTR) of a messenger RNA plays a pivotal role in transcriptional and translational regulation of gene expression. In particular, formation of large secondary structures mainly consisting of stem-loop structures located upstream of the initiation codon allows for allosteric regulation of mRNA expression of metabolic genes through direct binding to small-molecule effectors and proteins in response to the metabolic state of cells. In bacteria B. subtilis, tryptophan-induced direct binding of RNA-binding attenuation protein (TRAP) to the noncoding leader region of trp mRNAs controls the transcriptional and translational expression of tryptophan biosynthetic genes.

Similarly, riboswitches, an abundant type of highly structured metabolite-binding mRNA regulatory elements within the 5′-UTR, mainly terminate transcription or inhibit translation of downstream metabolic genes following conformational rearrangements within their aptamer domain that are triggered by direct binding to their corresponding target effectors, independent of protein factors. Thereby, given their efficacy as regulatory RNA modules controlling gene repression, riboswitch-based mechanisms remain an attractive concept for the generation of specific ligand-responsive synthetic mRNAs that can be customized for a wide range of metabolites.

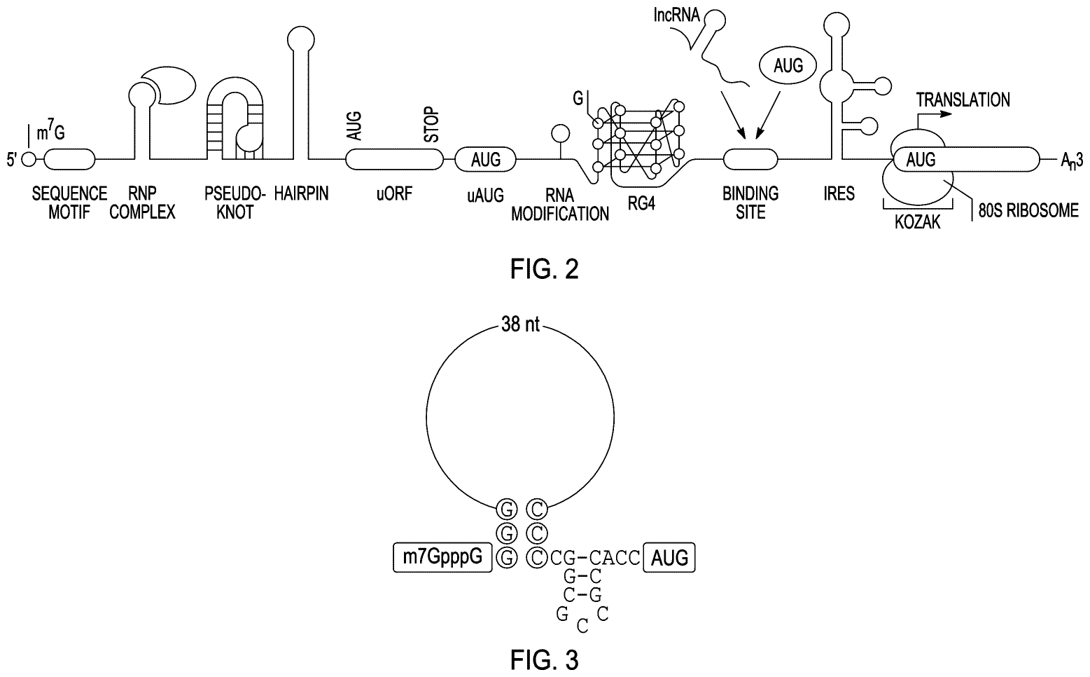

In metazoans, more detailed analysis of RNA segments at 5′-UTR using more recently developed integrative methodologies that allow for high precision assessment of mRNA structures inside cells suggests potential cis-acting regulatory RNA elements and structures with diverse modes of action as modulators of translation initiation (FIG. 2).

Among them, perhaps the first and most-studied examples of eukaryotic 5′-UTR structured element found to affect the translation is the small iron response elements (IREs) that folds into a single stem-loop and forms a by binding to the iron-regulatory proteins (IRP1 and IRP2) in response to low-iron condition. The resulting ribonucleoprotein (RNP) complex represses translation initiation of a subset of mRNAs involved in iron homeostasis by sterically hindering ribosome entry.

Overall structural assessment of cis-regulatory elements indicates that secondary and higher-order ternary mRNA structures (i.e., pseudoknots, G-quadruplex) within 5′UTR exert an inhibitory effect on translation initiation mainly by blocking cap-dependent ribosome entry. However, a more recent study revealed that partial-base pairing between an antisense long non-coding IncRNAs and its complementary mRNA 5′-UTR promotes ribosome binding and translation underscoring a yet less explored regulatory potential mechanism encoded in the structure-mediated sensing and recruitment of interacting factors such as RBPs or trans-acting RNAs at 5′UTR.

### Design Rationale and Implementation

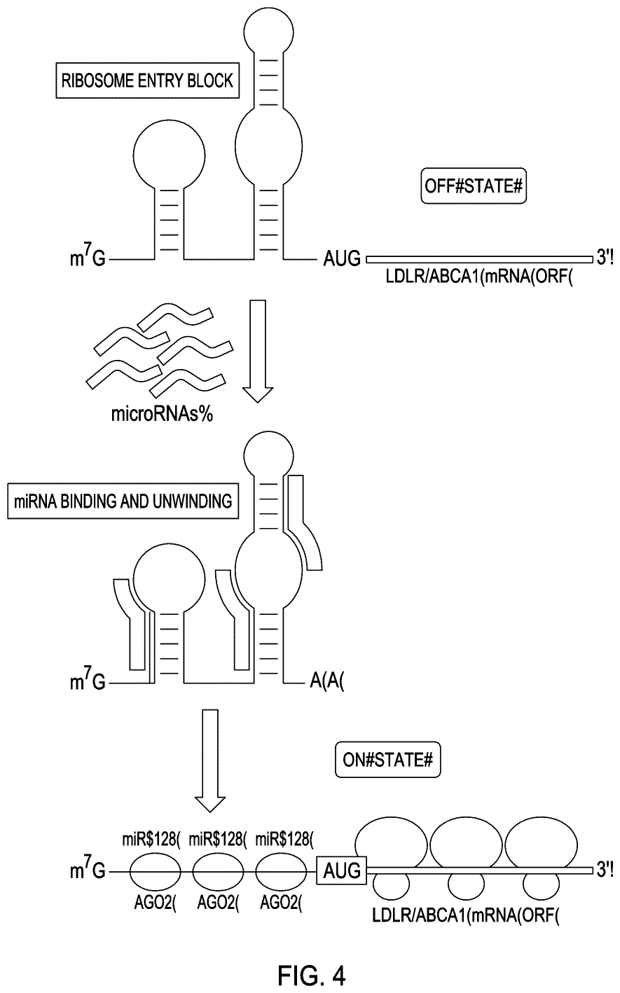

The kinetics of these events was investigated resulting in a design strategy that comprises an effector-regulated switch mechanism by generating a structured 5′-UTR operating in response to an endogenous mature microRNA sequence as further detailed below. A cap-dependent LDLR mRNA construct with a GC-poor 5′UTR for enhanced translational efficacy (FIG. 3) was used to generate nine different LDLR mRNA constructs containing miR-148a-binding sites as part of an extended 5′UTR. Thereby, the binding sites are incorporated within defined stem-loop structures, that are expected to sterically interfere with ribosome entry and translation initiation. As schematically depicted in FIG. 4, the translational initiation is effectively triggered upon miR-148 binding following a structural resolution of the hairpin-loop motifs.

The affinity between miR-148 and its binding sites at 5′UTR was altered in length to determine optimal miR-148 targeting efficacy that would cause the highest translational activation. This was archived by generating a minimum of three binding sites as part of two sequential hairpin stem-loop structures upstream of the initial 5′UTR. Thereby, m-fold secondary structure prediction program was employed to assess potential variations in secondary structure formation. The calculated negative folding free energy (AG) level was used as a measure for the robustness and stability of the resulting hairpin loops structures. Given RNA's potential to engage in intricate-base pairing patterns, single nucleotides within the flanking region of the binding sites were selectively altered to further stabilize the loop and the stem domains in order to avoid alternative secondary structure formation. This optimization step is mainly based on modulating the GC content in the 5′UTR without significantly reducing the level of -AG.

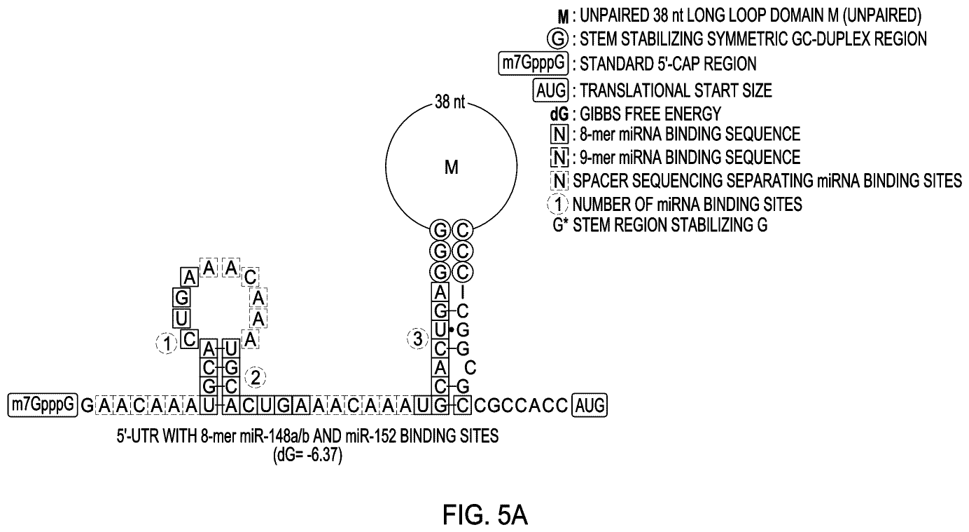

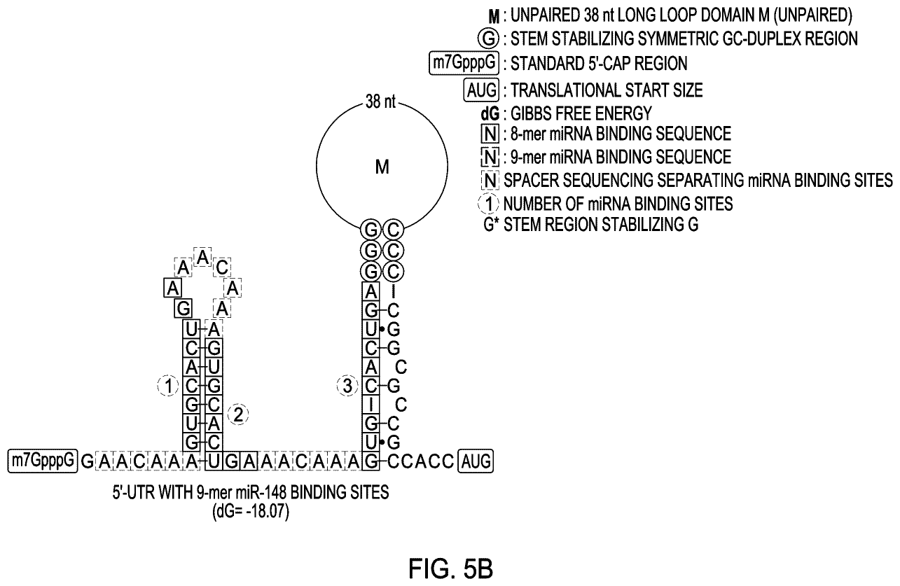

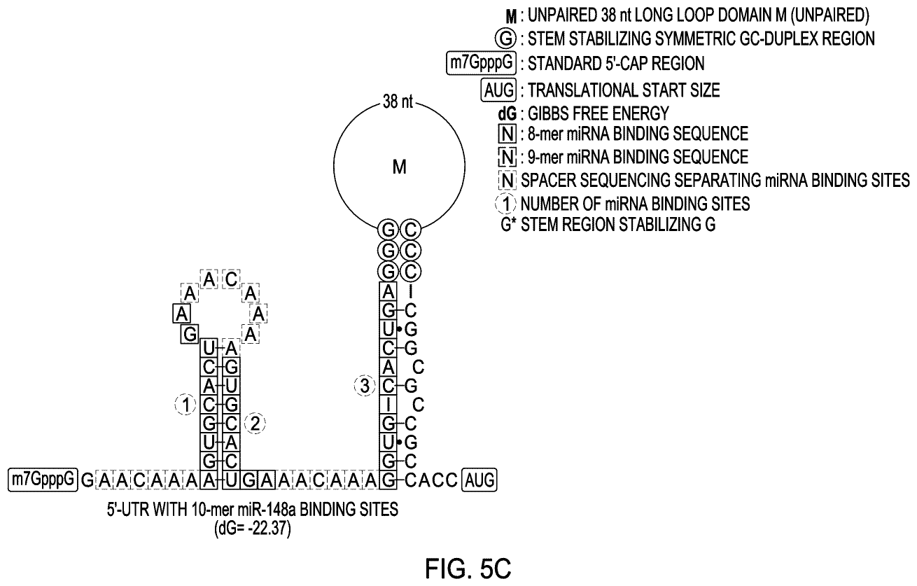

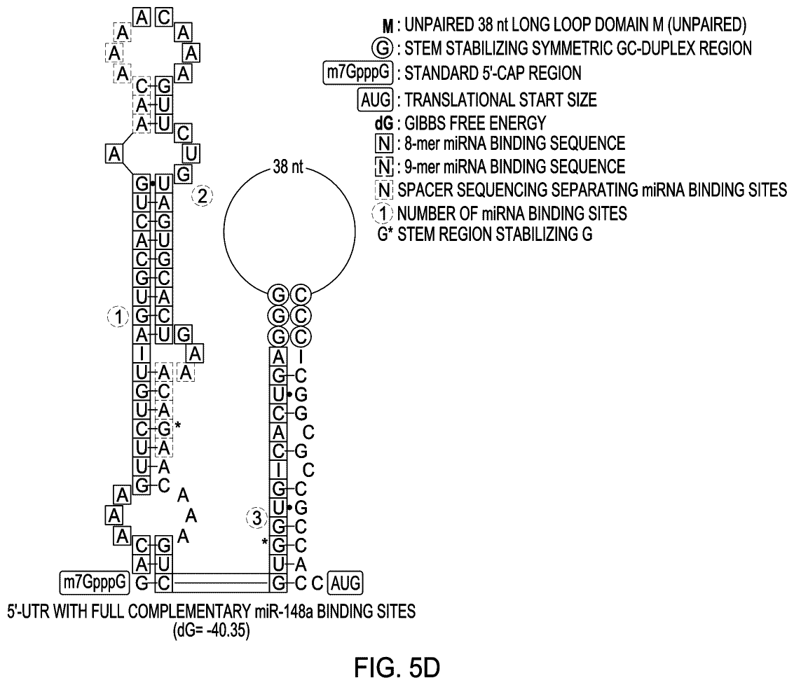

In this context, four constructs were generated that contained a minimum of three binding sites complementary to either full length or in partial to the first 8, 9 or 10 nucleotides of miR-148a (FIG. 5), hereafter referred to as C1-C4 constructs,

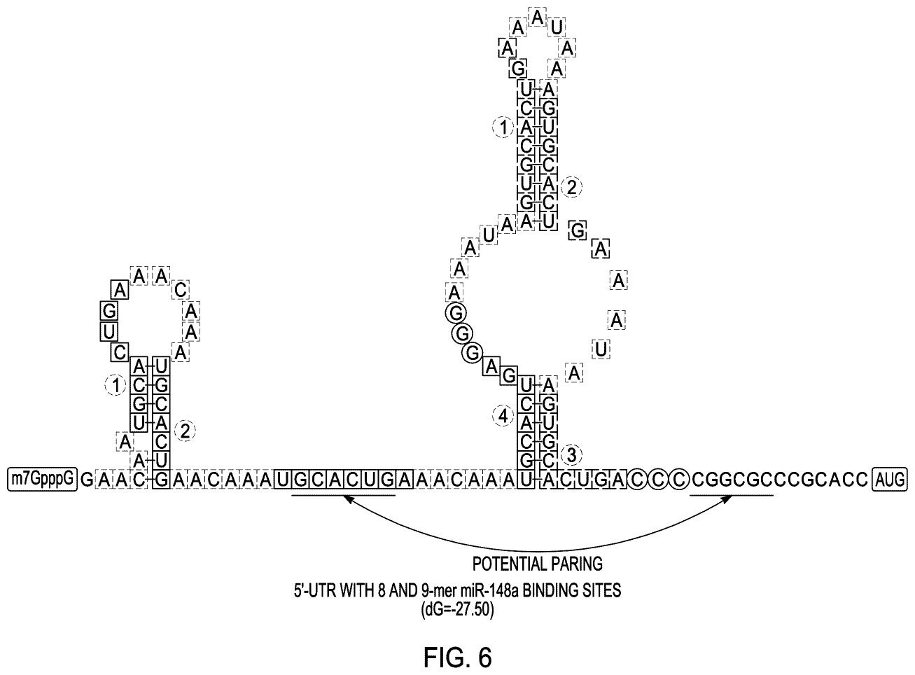

In addition, a fifth construct was generated that harbored a combination of four 8 and three 9-nt long binding sites for miR-148a to further increase the miR148a:mRNA interaction ratio leading to a stronger repression of miR-148 level, hence, higher endogenous LDLR and ABCA1 expression (FIG. 6).

In a more recent study, upon screening randomized sequences flanking each site of a microRNA binding site, David Bartel's lab at MIT showed that the microRNA targeting efficacy can be further enhanced by up to 100-fold using flanking A/U rich dinucleotides sequence. Based upon these finding, we decided to implement two major sets of flanking dinucleotides sequences (FDS#1 and #2) and designed four additional constructs harboring either 3 or 1 canonical 8-mer binding sites flanked by FDS#1 and 2, respectively.

### Expression Analysis of miR-148a regulated LDLR mRNA constructs In Vitro

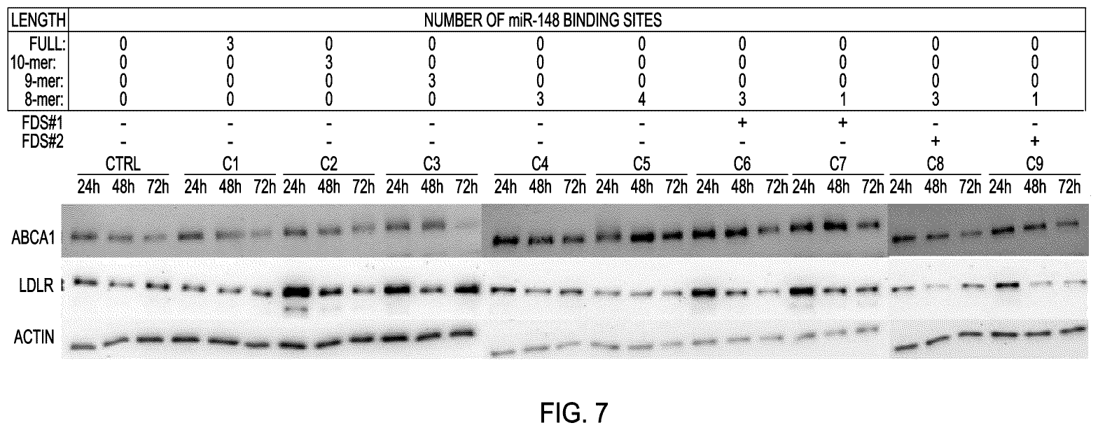

Next, the protein expression level of each construct was tested in hepatic carcinoma cells (HepG2) where miR-148a expression. As shown in FIG. 7, the expression of four (C2, C3, C6 and C7; see Table 3) out of nine tested constructs were significantly higher by 5-10-fold after 24-hours post-transfection as compared to the unmodified generic LDLR mRNA construct. Among them LDLR protein expression was highest in construct C2 treated cells consisting of three 10-mer binding sites. Notably the expression of the 8-mer containing constructs was highest in C6 and C7 in which the binding sites were specifically flanked by an “asymmetric” A/U rich dinucleotide sequence. Parallel assessment of ABCA1 expression also revealed that C5, with the highest number of miR-148 binding sites, significantly elevates ABCA1 expression at 48. However, C5 mediated LDLR mRNA translation remained relatively low most likely due to impaired translation initiation as a consequence of increased Argonaute-miRNA complex interference. A similar effect on ABCA1 expression was observed in C6 and C7 treated cells but with concomitant increase in LDLR expression. This can be partially explained by a relatively reduced number of 148a-binding sites and a more efficacious miR-148a targeting given the flanking dinucleotide sequences.

### Validation of the miR-148a Dependent Switch Mechanism

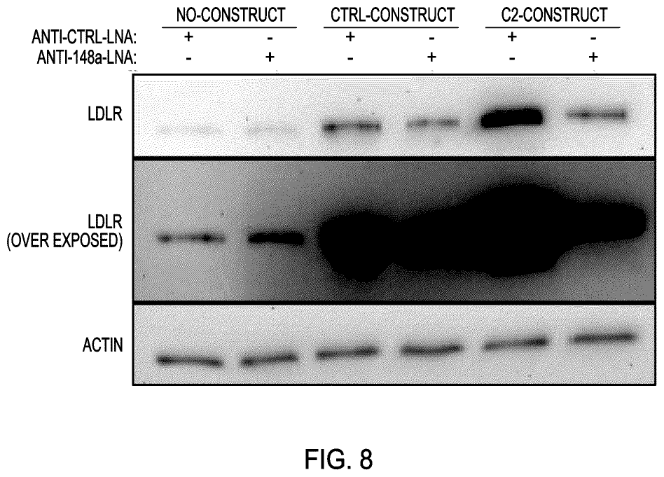

To show that the implemented switch mechanism in the engineered 5′UTRs is miR-148-dependent, loss of function studies were performed using LNA-antisense mediated depletion of endogenous miR-148a expression comparing the highly translated mRNA C2 and the control mRNA in HepG2 cells. As shown in FIG. 8, miR-148a inhibition by LNA antisense oligos significantly inhibited C2 mRNA translation without affecting the expression of the control LDLR mRNA indicating an intact switch mechanism that requires miR-148a as an effector molecule to turn on translation initiation. Of note, the. outcome of this, experiment provides the first example of an engineered regulatory mechanism that reverse microRNA functions in cells from classical translation inhibition to activation.

### Pre-clinical Assessment in LDLR KO Mice

To extend the duration of synthetic mRNA translation is prerequisite to their utilization as therapeutics for the treatment of metabolic diseases. To underscore the therapeutic potential of the highly translated mRNAs C2, C3, C6 and C7 in lowering LDL-cholesterol, LDLR KO mice (n=5 per group) kept on western type of diet (16% fat, 0.15% cholesterol) for 28 days were treated with a single of dose 0.5 pkm and their serum cholesterol and liver protein levels assessed after a 4 days intervention period.

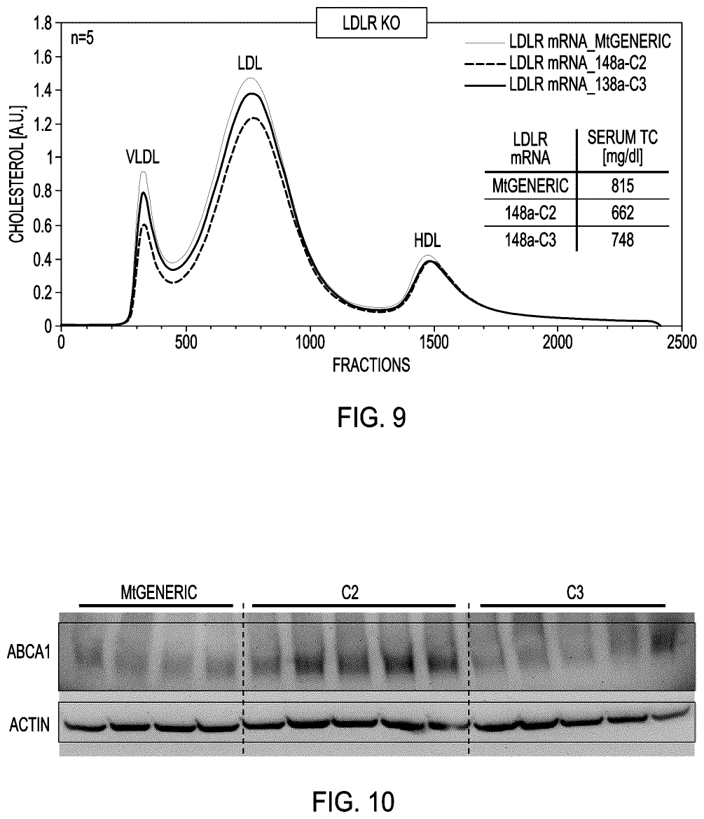

In accord with the effect obtained from cell culture studies, C2 significantly reduced non-HDL-cholesterol levels as compared to generic LDLR mRNAs in severely hypercholesteremic male LDLR KO mice (FIG. 9). Thereby, C2 treatment led to reduced VLDL and LDL-cholesterol content without altering HDL levels as shown by FPLC analysis of the lipoprotein distribution profile.

Moreover, C2 but not C3 expression was associated with increased hepatic ABCA1 expression potentially due to miR-148 binding, a positive effect that can elevate HDL levels in long-term (FIG. 10).

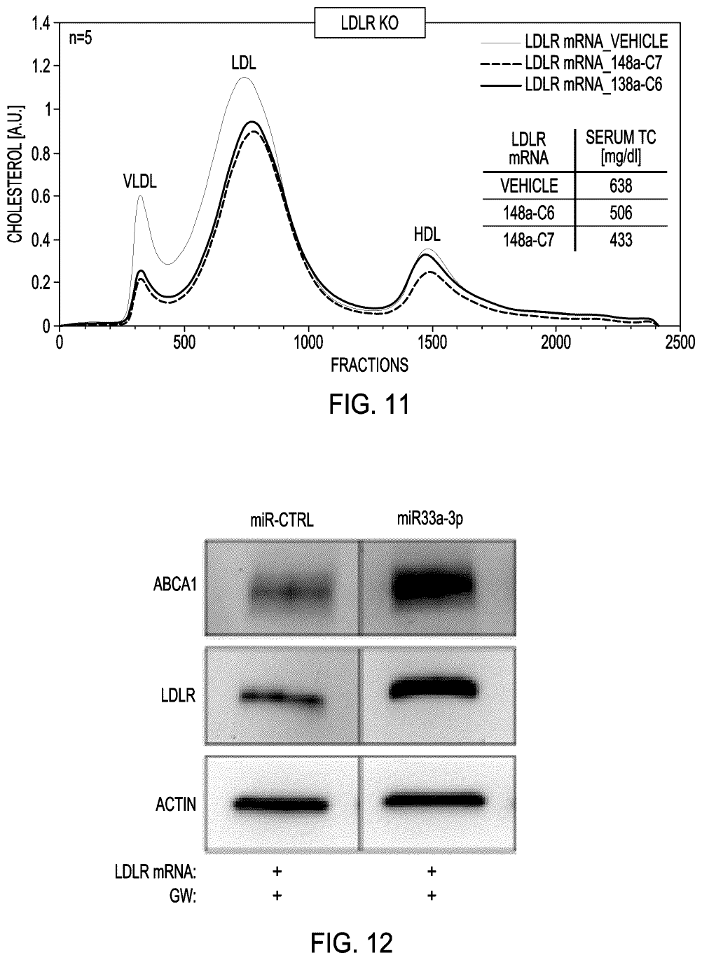

Similarly, treatment of female LDLR KO mice with C6 and C7 constructs resulted in significantly reduced total cholesterol levels up to about 30% after 4 days by a single dose administration (FIG. 11).

### EXAMPLE 2

For a cholesterol regulating pathway as fundamental as LDL uptake, it is appreciated that there are redundant circuits to ensure sufficient low-density lipoprotein (LDL) receptor (LDLR) activity. miR-33a, embedded within intronic sequence of the sterol regulatory element-binding protein-2 (SREBP-2) not only complement the function of its host gene to boost intracellular cholesterol levels by inhibiting the ABCA1 mediated cholesterol efflux but also acts to promote LDLR mediated removal of atherogenic LDL from circulation. This is achieved by miR-33a directed inhibition of PCSK9 and two LXR target genes known as IDOL and ANGPTL3 that negatively influence LDLR expression. LDL-cholesterol and VLDL-triglyceride lowering strategies were developed by utilizing liver-targeted delivery of miR-33a-3p mimics into various diet induced obese mouse models and it was found that reduced hepatic and circulating PCSK9 levels significantly lowered LDL ameliorated hepatic steatosis secondary to increased transient VLDL secretion, as well as long-term plasma TG reduction.

Notably, it was found that miR-33a-3p enhances the stability of LDLR mRNA constructs in the presence of GW-induced LXR activity with concomitant increase in ABCA1 expression in HepG2 hepatocytes (FIG. 12). The therapeutic efficacy of miR-33a mimics in combination with selected LDLR mRNA constructs may allow for enhancement of LDLR integrity.

Thus, targeted RNA-based therapies can be developed with higher efficacy for the treatment of FH. Thereby, using endogenous microRNAs as effector-molecules to selectively activate mRNA translation provide a new tool, beside its conceptually novel mechanism, for design and generation of tissue/cell specific mRNA expression. Taken together, a modular platform was established that benefits from both microRNAs and mRNAs as potential therapeutic targets. Thus, synthetic switchable LDLR mRNA constructs may be employed as new drugs for the treatment of hypercholesterolemia as other related cardiometabolic diseases.

### EXAMPLE 3

Following the analysis of the initially designed constructs, the translational efficacy of other constructs (see Table 3) was tested by combining sequence features incorporated into C2 and C6/7 that might have contributed to higher LDLR protein level that were observed.

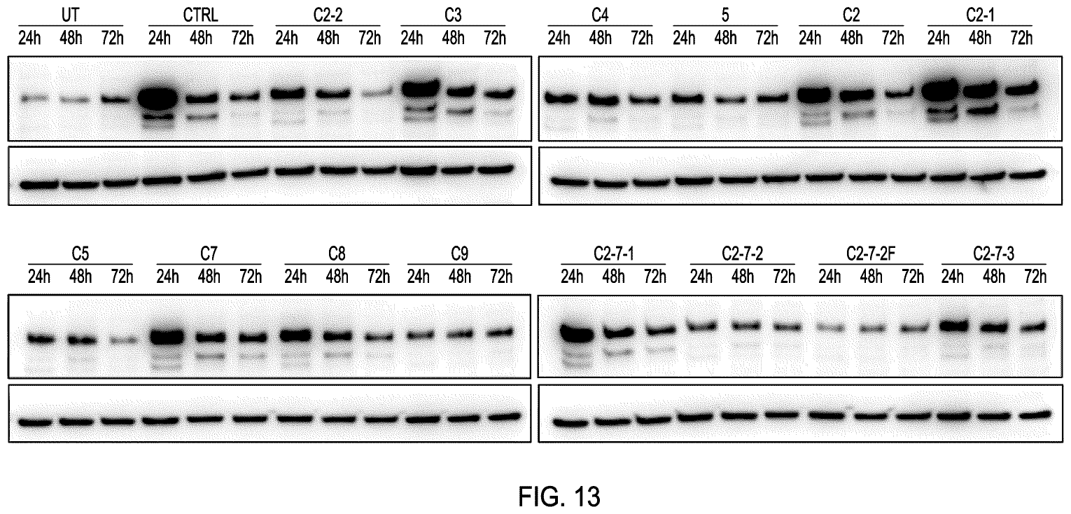

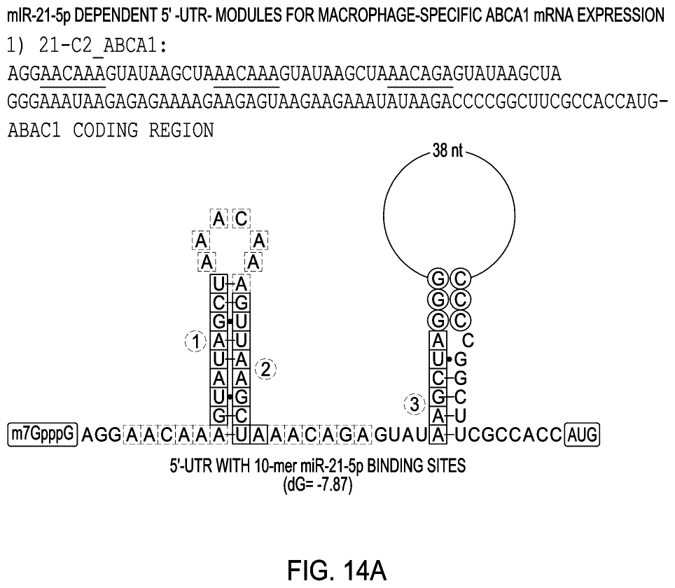

As shown in FIG. 13, it was found that reducing the number of miR-148 binding sites in C2 from three 10-mers down to 1 site, as in C7, resulted in significantly higher LDLR protein levels up to 72 hours in HepG2 cells under standard cell conditions as shown for C2-1. In addition, it was found that reducing the length of the binding sites from 10-mer down to 8-mers may have a negative effect on translational efficacy of the constructs. However, translational efficacy was independent of the length of the binding site in constructs harboring one single binding site (C7 vs C2-7-1). It was also found that translation efficacy of the constructs may be negatively affected in response to increased integration of flanking sequences as systematically shown for C2-7-1 vs C2-7-2 vs C2-7F. Notably, multiple incorporation of binding sites of 8-nucleotides in length (8-mers) resulted in a higher protein level as shown for C8 compared to C9.

Taken together, the miR-148 responsive LDLR mRNA constructs allowed for the generation of mRNA constructs for tissue targeted expression, e.g., LDLR mRNA-based “drug” for the treatment of FH. In particular, C2-1 has desirable properties

TABLE 3
LDLR-Anti-miR148 5′UTR Chimeras (IDOL and PCSK9 resistant)

C-2
GGGAACAAAAGUGCACUGAAACAAAAGUGCACUGAAACAGAGGUGCACUGAGGGAAAUAAGAG
AGAAAAGAAGAGUAAGAAGAAAUAUAAGACCCCGGCGCCGCCACCAUG (SEQ ID NO: 107)
C-2/1
GGGAACAAAAGUGCACUGAGGGAAAUAAGAGAGAAAAGAAGAGUAAGAAGAAAUAUAAGACCC
CGGCGCCGCCACCAUG (SEQ ID NO: 108)
C-2/2
GGGAACAAAAGUGCACUGAAACAAAAGUGCACUGAGGGAAAUAAGAGAGAAAAGAAGAGUAAG
AAGAAAUAUAAGACCCCGGCGCCGCCACCAUG (SEQ ID NO: 109)
C-3
GGGAACAAAGUGCACUGAAACAAAGUGCACUGAAACAGAGUGCACUGAGGGAAAUAAGAGAGA
AAAGAAGAGUAAGAAGAAAUAUAAGACCCCGGCGCCGCCACCAUG (SEQ ID NO: 110)
C-4
GGGAACAAAUGCACUGAAACAAAUGCACUGAAACAAAUGCACUGAGGGAAAUAAGAGAGAAAA
GAAGAGUAAGAAGAAAUAUAAGACCCCGGCGCCGCCACCAUG (SEQ ID NO: 111)
5
GGGAACAAUUGCACUGAAACAAAUGCACUGAAACAAAUGCACUGAAACAAAUGCACUGAGGGA
AAUAAGUGCACUGAAAAUAAGUGCACUGAAAAUAAGUGCACUGACCCCGGCGCCGCCACCAUG
(SEQ ID NO: 112)
C-5
AGGAACAAUCUGCACUGAAACAAACUGCACUGAAACAAACUGCACUGAAACAAACUGCACUGAG
GGAAAUAAGUGCACUGAAAAUAAGUGCACUGAAAAUAAGUGCACUGACCCCGGCGCCGCCACC
AUG (SEQ ID NO: 113)
C7
AGGAAAAGUGCACUGAAUUUAGGGAAAUAAGAGAGAAAAGAAGAGUAAGAAGAAAUAUAAGAC
CCCGGCGCCGCCACCAUG (SEQ ID NO: 114)
C-2/7-1
AGGAAAAGAGUGCACUGAAUUUAGGGAAAUAAGAGAGAAAAGAAGAGUAAGAAGAAAUAUAAG
ACCCCGGCGCCGCCACCAUG (SEQ ID NO: 115)
C-2/7-2
AGGAAAAGAGUGCACUGAAUUUAAAAAGAGUGCACUGAGGGAAAUAAGAGAGAAAAGAAGAGU
AAGAAGAAAUAUAAGACCCCGGCGCCGCCACCAUG (SEQ ID NO: 116)
C-2/7-2F
AGGAAAAGAGUGCACUGAAUUUAAAAAGAGUGCACUGAAUUUAGGGAAAUAAGAGAGAAAAGA
AGAGUAAGAAGAAAUAUAAGACCCCGGCGCCGCCACCAUG (SEQ ID NO: 117)
C-2/7-3
AGGAAAAGAGUGCACUGAAUUUAAAAAGAGUGCACUGAAUUUAAAAAGAGUGCACUGAGGGAA
AUAAGAGAGAAAAGAAGAGUAAGAAGAAAUAUAAGACCCCGGCGCCGCCACCAUG (SEQ ID
NO: 118)
C-8
AGGAAUAAACUGCACUGAAAUAAACUGCACUGAAAUAAACUGCACUGAAAUAAAGGGAAAUAAG
AGAGAAAAGAAGAGUAAGAAGAAAUAUAAGACCCCGGCGCCGCCACCAUG (SEQ ID NO: 119)
C-9
AGGAAUAAACUGCACUGAAAUAAAGGGAAAUAAGAGAGAAAAGAAGAGUAAGAAGAAAUAUAA
GACCCCGGCGCCGCCACCAUG (SEQ ID NO: 120)

### EXAMPLE 4

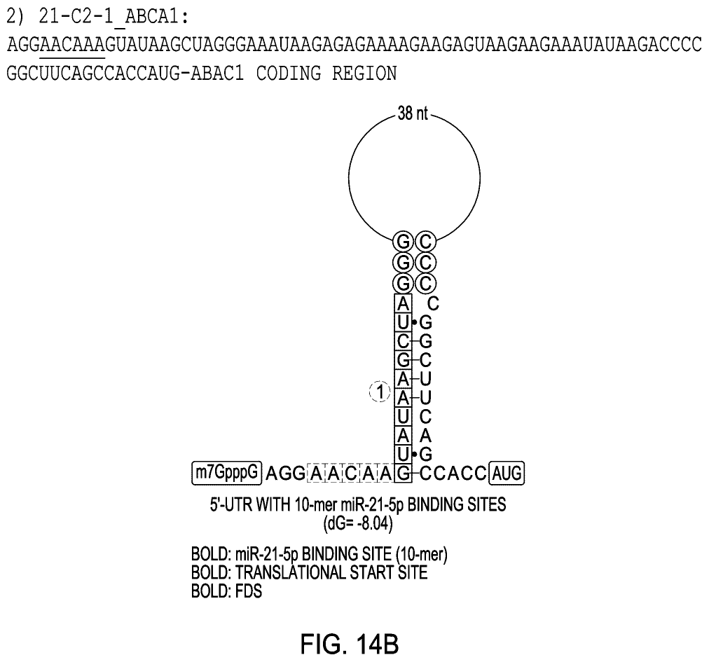

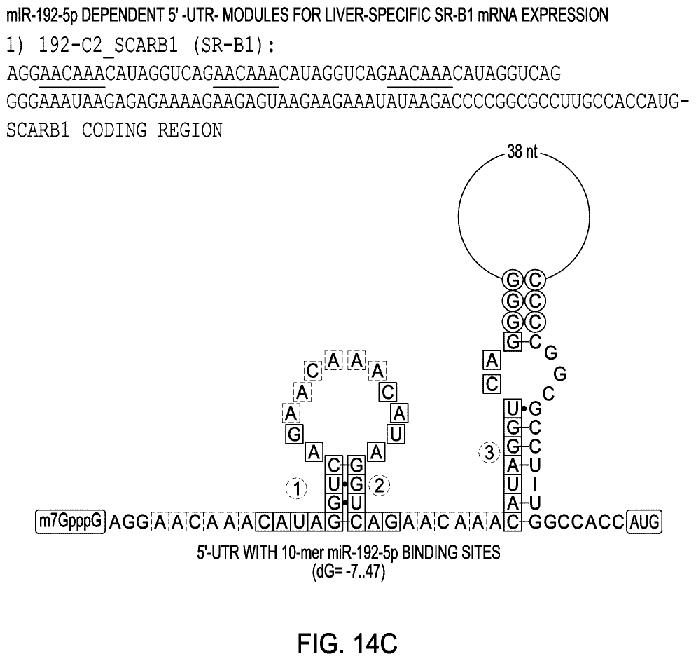

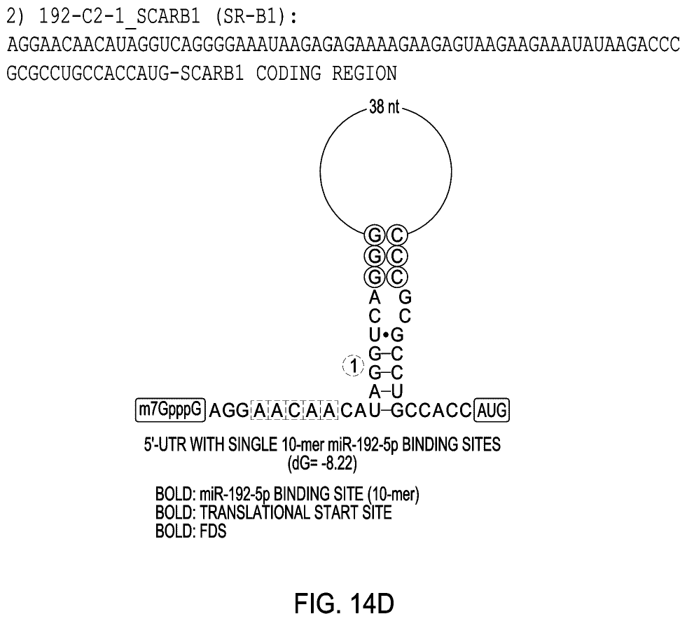

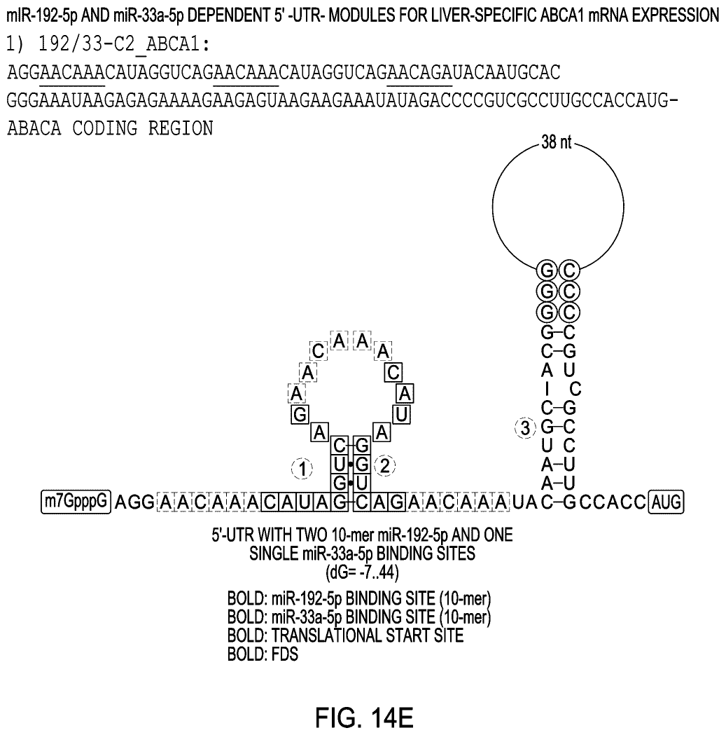

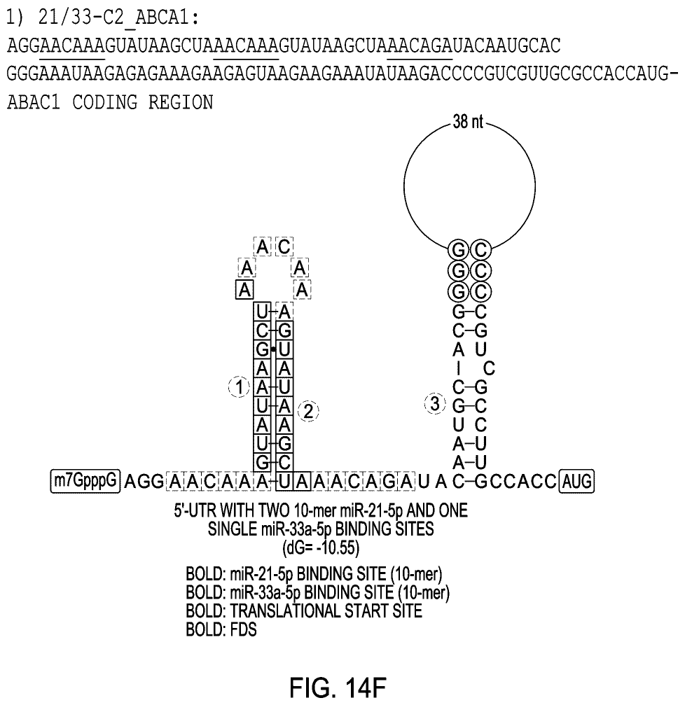

Other constructs (FIG. 14) were prepared, e.g., 5′UTR modules based on the C2 and C2-1 backbone for macrophage and liver-specific expression of HDL-generating cholesterol offlux pump ABCA1 and the HDL binding receptor SR-B1. miR-192-5p binding sites were employed as miRNA192-6p expression is specific and significantly higher in liver based on recent findings.

21-C2_ABCA1:
(SEQ ID NO: 121)
AGGAACAAAGUAUAAGCUAAACAAAGUAUAACCUAAACAGAGUADAAGCUA
GGGAAAUAAGAGAGAAAAGAAGAGUAAGAAGAAAUAUAAGACCCCGGCUUCGCCACCAUG-
ABAC1 coding region
21-C2-1_ABCA1:
(SEQ ID NO: 122)
AGGAACAAAGUAUAAGCUAGGGAAAUAAGAGAGAAAAGAAGAGUAAGAAGAAAUAUAAGACCCCG
GCUUCAGCCACCAUG-ABAC1 coding region

Bold: miR-21-5p binding site (10-mer)
Bold: Translational start site
Bold: FDS

In addition, 5′UTRs were designed for ABCA1 that also harbor a binding site for miR-33 next to either binding sites for miR-21 or 192 for macrophage and liver expression, respectively. miR-33 is an endogenous inhibitor of ABCA1 expression, and these constructs can eliminate miR-33 to further boost ABCA1 expression, while their binding to miR-21 or miR-192 controls their tissue/cell specific expression.

### miR-192-5p Dependent 5′-UTR-Modules for Liver-Specific SR-B1 mRNA Expression

192-C2_SCARB1 (SR-B1):
(SEQ ID NO: 123)
AGGAACAAACAUAGGUCAGAACAAACAUAGGUCAGAACAAACAUAGGUCAG
GGGAAAUAAGAGAGAAAAGAAGAGUAAGAAGAAAUAUAAGACCCCGGCGCCUUGCCACCAUG-
SCARB1 coding region
192-C2-1_SCARB1 (SR-B1):
(SEQ ID NO: 124)
AGGAACAAACAUAGGUCAGGGGAAAUAAGAGAGAAAAGAAGAGUAAGAAGAAAUAUAAGACCC
GCGCCUGCCACCAUG-SCARB1 coding region

Bold: miR-192-5p binding site (10-mer)
Bold: Translational start site
Bold: FDS

### miR-192-5p and miR-33a-5p Dependent 5′-UTR-Modules for Liver-Specific ABCA1 mRNA Expression

192/33-C2_ABCA1:
(SEQ ID NO: 125)
AGGAACAAACAUAGGUCAGAACAAACAUAGGUCAGAACAGAUACAAUGCAC
GGGAAAUAAGAGAGAAAAGAAGAGUAAGAAGAAAUAUAAGACCCCGUCGCCUUGCCACCAUG-
ABAC1 coding region

Bold: miR-192-5p binding site (10-mer)
Bold: miR-33a-5p binding site (10-mer)
Bold: Translational start site
Bold: FDS

### miR-21-5p and miR-33a-5p Dependent 5′-UTR-Modules for Macrophage-Specific ABCA1 mRNA Expression

21/33-C2_ABCA1:
AGGAACAAAGUAUAAGCUAAACAAAGUAUAAGCUAAACAGAUACAAUGCAC
GGGAAAUAAGAGAGAAAAGAAGAGUAAGAAGAAAUAUAAGACCCCGUCGUUGCGCCACCAUG

### (SEQ ID NO: 126) -ABAC1 Coding Region

Bold: miR-21-5p binding site (10-mer)
Bold: miR-33a-5p binding site (10-mer)
Bold: Translational start site
Bold: FDS

The following statements are intended to describe and summarize various features of the invention according to the foregoing description provided in the specification and figures.

### STATEMENTS

1. Isolated mRNA comprising a 5′UTR, a coding sequence optionally encoding a gene product, and a poly A sequence, wherein the 5′UTR comprises a nucleotide sequence that forms a stem loop structure, wherein the stem comprises a microRNA binding site or a portion thereof.
2. The isolated mRNA of statement 1 wherein the microRNA is expressed in a tissue or cell specific manner.
3. The isolated mRNA of statement 1 or 2 wherein the 5′UTR further comprises a second nucleotide sequence that forms a stem loop structure.
4. The isolated mRNA of statement 3 wherein the second nucleotide sequence comprises a further microRNA binding site
5. The isolated mRNA of statement 1, 2, 3 or 4 wherein the 5′UTR has a G-C poor sequence.
6. The isolated mRNA of statement 5 wherein the 5′UTR has less than 50%, 40%, 30%, 20% or 10% G-C content.
7. The isolated mRNA of any one of statements 1 to 6 which comprises two or more microRNA binding sites.
8. The isolated mRNA of any one of statements 1 to 7 which comprises three, four, five, six, seven, eight, nine or more microRNA binding sites.
9. The isolated mRNA of statement 7 or 8 wherein at least one microRNA binding site is not part of a stem loop.
10. The isolated mRNA of any one of claims 1 to 9 wherein the microRNA binding site is 30, 25, 20, 15, 14, 13, 12, 11, 10, 9, 8, 7, 6, or 5 nucleotides in length,
11. A vector comprising a nucleic acid sequence encoding the mRNA of any one of statements 1 to 10.
12. The vector of statement 11 which is a plasmid.
13. The vector of statement 11 which is a viral vector.
14. A method to alter expression of a gene product in a mammalian cell, comprising; contacting the cell with an effective amount of the isolated mRNA of any one of statements 1 to 10 or the vector of any one of statements 11 to 13.
15. The method of statement 14 wherein the mammal is a human.
16. The method of statement 14 or 15 wherein the cell is in a mammal.
17. The method of statement 14, 15 or 16 wherein at least one microRNA binding site is flanked by at least one A/U rich sequence in the mRNA.
18. The method of statement 17 wherein the A/U rich flanking sequences are asymmetric.
19. The method of statement 17 or 18 wherein the A/U rich sequence has at least 4, 5, 6, 7, 8, 9, 10 or more nucleotides.
20. The method of any one of statements 17 to 19 wherein the A/U rich sequence has one, two, three or four G or C nucleotides.
21. The method of any one of statements 17 to 20 wherein the A/U rich sequence has at least 4, 5, 6, 7, 8, 9, 10 or more A or U/T nucleotides.
22. A method to prevent, inhibit or treat hypercholesterolemia, to lower non-HDL-cholesterol levels or to prevent, inhibit or treat atherosclerosis in a mammal, comprising: administering to a mammal in need thereof an effective amount of a composition comprising a nucleic acid sequence comprising a 5′UTR comprising at least one miRNA binding site for a miRNA expressed in the liver and an open reading frame encoding a gene product the expression of which in the mammal prevents, inhibits or treats hypercholesterolemia or lowers non-HDL-cholesterol levels, wherein expression of gene product is enhanced relative to a mammal not administered the composition or administered a composition comprising a corresponding nucleic acid sequence that lacks the at least one miRNA binding site.
23. The method of statement 22 wherein the at least one miRNA binding site binds miR148, miR33, miR122 or miR223.
24. The method of statement 22 or 23 wherein the at least one miRNA binding site has one of SEQ ID Nos. 16 to 18.
25. The method of statement 22, 23 or 24 wherein the 5′UTR comprises one of SEQ ID Nos. 1-30, 40-45, 60-66, 80-86, 104, 105 or 107-126.
26. The method of any one of statements 22 to 25 wherein the mammal is a human.
27. The method of any one of statements 22 to 26 wherein the composition comprises liposomes.
26. The method of statements 27 wherein the liposomes comprise or more of DC-cholesterol, 1,2-dioleoyl-sn-glycero-3-phosphoethanolamine (DOPE), an ionizable cationic lipid or a lipidoid.
27. The method of any one of statements 22 to 26 wherein the composition comprises nanoparticles.
28. The method of any one of statements 22 to 27 wherein the composition is systemically administered.
29. The method of any one of statements 22 to 27 wherein the composition is orally administered.
30. The method of any one of statements 22 to 28 wherein the composition is injected.
31. The method of any one of statements 22 to 30 wherein the at least one miRNA binding site is 10 nucleotides or less in length.
32. The method of any one of statements 22 to 31 wherein the nucleic acid sequence comprises non-native nucleotides.
33. The method of statement 32, wherein the non-native nucleotide has a modified nucleobase, modified phosphate group or a modified sugar.
34. The method of statement 33, wherein the non-native nucleotide comprises one or more modified phosphodiester binds, phosphorothioate bonds, 2′-O-methyl, 2′-O-methoxyethyl, 2′-fluoro, locked nucleic acid (LNA), 5′ vinylphosphonate, 5 -formyl cytidine or pseudouridine.
35. The method of any one of statement 22 to 34, wherein the amount of the nucleic acid sequence is about 0.01 mg/kg to about 100 mg/kg, about 0.05 mg/kg to about 10 mg/kg, about 10 mg/kg to about 75 mg/kg, or about 1 mg/kg to about 100 mg/kg.
36. The mRNA of any one of statements 1 to 10, the vector of any one of statements 11 to 13 or the method of any one of statements 14 to 34 wherein the open reading frame encodes LDLR or ABCA1.

All publications, patents and patent applications are incorporated herein by reference. While in the foregoing specification, this invention has been described in relation to certain preferred embodiments thereof, and many details have been set forth for purposes of illustration, it will be apparent to those skilled in the art that the invention is susceptible to additional embodiments and that certain of the details herein may be varied considerably without departing from the basic principles of the invention.

The specific methods and compositions described herein are representative of some embodiments and are exemplary and not intended as limitations on the scope of the invention. Other objects, aspects, and embodiments will occur to those skilled in the art upon consideration of this specification and are encompassed within the spirit of the invention as defined by the scope of the claims. It will be readily apparent to one skilled in the art that varying substitutions and modifications may be made to the invention disclosed herein without departing from the scope and spirit of the invention.

Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims and statements of the invention.

1. Isolated mRNA comprising a synthetic 5′UTR, a coding sequence for a therapeutic or prophylactic gene product, and a poly A sequence, wherein the synthetic 5′UTR comprises a nucleotide sequence that forms at least a first stem loop or hairpin structure, wherein the at least first stem or portion of the hairpin comprises a first microRNA (miRNA) binding site or a portion thereof, the presence of which in the mRNA results in enhanced translation of the gene product relative to corresponding mRNA that lacks the nucleotide sequence.

2. The isolated RNA of claim 1 wherein the synthetic 5′UTR comprises at least a second stem loop or hairpin structure which comprises at least one other miRNA binding site and which optionally is 3′ to the first stem loop or hairpin structure.

3. The isolated mRNA of claim 1 wherein the at least first stem or hairpin portion comprising the at least first miRNA binding site, or the second stem or hairpin, includes a complementary sequence of the miRNA binding site that results in a mismatch of one or more nucleotides.

4. The isolated mRNA of claim 3 wherein the mismatch includes 2, 3, 4 or 5 mismatches.

5. The isolated mRNA of claim 1 wherein the synthetic 5′UTR is 200 nucleotides or less in length.

6.-8. (canceled)

9. The isolated mRNA of claim 1 wherein the miRNA is expressed in a tissue- or cell-specific manner.

10. The isolated mRNA of claim 1 wherein the synthetic 5′UTR further comprises a second nucleotide sequence that forms a stem loop structure and is 3′ to the nucleotide sequence comprising the at least first miRNA binding site.

11. The isolated mRNA of claim 10 wherein the second nucleotide sequence comprises a further miRNA binding site which optionally is for the same miRNA as the first miRNA binding site.

12. (canceled)

13. The isolated mRNA of claim 1 wherein the synthetic 5′UTR has less than 50%, 40%, 30%, 20% or 10% G-C content.

14. The isolated mRNA of claim 1 which comprises three or fewer miRNA binding sites.

15. (canceled)

16. The isolated mRNA of claim 14 which comprises one or two miRNA binding sites for one miRNA and at least on other miRNA binding site for a different miRNA.

17. (canceled)

18. The isolated mRNA of claim 1 wherein the 5′UTR comprises one of SEQ ID Nos. 1-36, 40-45, 60-66, or 80-86, or a nucleic acid sequence with at least 80%, 82%, 85%, 87%, 90%, 92%, 95%, 97%, 98% or 99% nucleotide sequence identity thereto.

19. (canceled)

20. A vector comprising a nucleic acid sequence encoding the mRNA of claim 1.

21. The vector of claim 20 which is a plasmid or viral vector.

22.-23. (canceled)

24. A method to alter expression of a gene product in a mammalian cell, comprising; contacting the cell with an effective amount of a composition comprising the isolated mRNA of claim 1.

25. The method of claim 24 wherein the mammal is a human.

26. The method of claim 24 wherein the cell is in a mammal.

27. The method of claim 24 wherein the composition comprises liposomes or nanoparticles.

28. (canceled)

29. The method of claim 24 wherein the composition is systemically administered.

30. (canceled)

31. The method of claim 24 wherein the composition is injected.

32. (canceled)
